(19) United States
(12) Patent Application Publication
(10) Pub. No.: US 2026/0263476 A1
(43) Pub. Date: Sep. 10, 2026

(54) SALTS AND CRYSTALLINE FORMS OF LM11A31 AND METHODS OF USE THEREOF

(71) Applicant: PharmatrophiX, Inc., Menlo Park, CA (US)

(72) Inventors: Frank M. Longo, Menlo Park, CA (US); Joan Benet Congost, Barcelona (ES); Raquel Enrech López, Barcelona (ES); Víctor Díaz Pérez, Barcelona (ES); Stephen Winter, Barcelona (ES); Jaimie Karyn Rueter, Framingham, MA (US)

(21) Appl. No.: 19/653,997

(22) Filed: Apr. 21, 2026

Related U.S. Application Data
(63) Continuation of application No. PCT/US25/61731, filed on Dec. 30, 2025.
(60) Provisional application No. 63/740,209, filed on Dec. 30, 2024.

(51) Int. Cl. A61K 31/5375; A61P 25/28; C07D 295/13

(52) U.S. Cl. CPC A61K 31/5375; A61P 25/28; C07D 295/13

(57) ABSTRACT

The disclosure provides, in part, a compound wherein the compound is a salt of a compound of Formula (I):

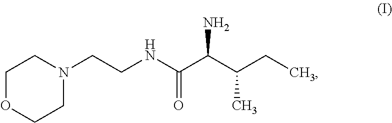 [SMILES: CC[C@H](C)[C@H](N)C(=O)NCCN1CCOCC1]

wherein the salt is an adipic salt or gentisic salt. Also provided herein is a crystalline form of a salt of a compound of Formula (I).

### CROSS-REFERENCE TO RELATED APPLICATIONS

This application claims the benefit of and priority to U.S. Provisional Application No. 63/740,209, filed on Dec. 30, 2024, the contents of which are hereby incorporated by reference herein in their entirety for all purposes.

### BACKGROUND

Neurotrophins are polypeptides that play a role in the development, function, and/or survival of certain cells, including neurons. The death, degeneration or dysfunction of neurons has been directly implicated in a number of neurodegenerative disorders, e.g., Alzheimer's Disease.

A variety of neurotrophins have been identified, including Nerve Growth Factor (NGF), Neurotrophin-3 (NT-3), Neurotrophin-4/5 (NT-4/5), Neurotrophin 6 (NT-6) and Brain Derived Neurotrophic Factor (BDNF) that interact principally with p75^{NTR}, which is coupled to pro-survival signaling systems, e.g., pro-apoptotic or pro-degenerative signals.

Small molecule agents with favorable drug-like features based upon neurotrophin domains that interact with neurotrophin receptors, that are capable of targeting and modulating specific neurotrophin receptors, may be useful in the treatment of disorders or diseases. For example, LM11A-31 (the compound represented by Formula (I)) is a promising therapeutic candidate currently in clinical trials for the treatment of Alzheimer's Disease.

### SUMMARY

The present disclosure provides new forms (e.g., salts and crystalline forms), and preparations of LM11A-31, which is a small chemical compound having the structure set forth in Formula (I):

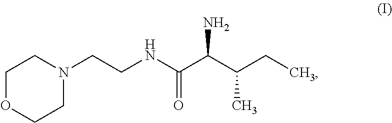 [SMILES: CC[C@H](C)[C@H](N)C(=O)NCCN1CCOCC1]

as well as various associated technologies including, for example pharmaceutical compositions that are prepared from and/or include such forms (and/or preparations), methods of using and/or manufacturing such forms, preparations, and/or compositions, including by crystallizing (or recrystallizing) a compound of Formula (I).

LM11A-31 is a p75^{NTR} modulator; LM11A-31 downregulates degenerative signaling mediated by p75^{NTR}, and furthermore acts as antagonist to pro-NGF-induced degeneration. LM11A-31 crosses the blood-brain barrier following oral administration, and is currently being pursued as a promising therapeutic candidate for treatment of Alzheimer's Disease. See, for example, Shanke et. al., Nat. Med 30:1761, May 17, 2024.

Among other things, the present disclosure identifies the source of a problem with presently available forms of LM11A-31, specifically including with the bis-hydrogen sulfate (BHS) sulfate form utilized in the Alzheimer's Disease clinical trials. Ibid; see also WO2011060262 and/or WO2014052659.

For example, the present disclosure identifies hygroscopicity and/or stability liabilities of such forms. The present disclosure further demonstrates that most other salts of LM11A-31, including those typically studied or utilized in pharmaceutical development, suffer similar liabilities. The present disclosure surprisingly identifies certain unorthodox salts of LM11A-31 with remarkably distinctive such properties, rendering these salts particularly useful in pharmaceutical compositions and/or in their manufacture or use. In some embodiments, the present disclosure teaches particular salts of LM11A-31 that are surprisingly useful in manufacturing of drug products at commercial scales and/or in storage of drug products over a range of conditions and/or for extended periods of time.

In particular embodiments, the present disclosure further provided specific crystal forms of certain LM11A-31 salts, and documents surprising usefulness of such forms in various contexts.

Still further, in certain embodiments, the present disclosure provides preparations of particular LM11A-31 salts, and/or particular crystal forms thereof, with distinctive and, in some embodiments, particularly useful characteristics; the present disclosure further provides technologies for manufacturing such salts, crystal forms, and/or preparations, and for using such preparations in the production of pharmaceutical compositions.

The present disclosure appreciates that p75^{NTR} has been reported to be involved in multiple biological pathways, including, for example, synaptic integrity, pruning and function (see, for example, Wong et al., Aging Cell 20:e13305 e13305, 2021; Yang et al., Transl. Psych. 11:578, 2021); tau phosphorylation (see, for example, Yang et al., Sci. Rep. 10:20322, 2020); inflammation (Dusedau et al., Glia 67:193, 2019); mitochondrial function (Tan et al., Curr. Neurovasc. Res. 18: 552, 2021; Meeker et al., Exp. Neurol. 275:182, 2016); amyloid production (Saadipour ey al., J. Neurochem. 144:302, 2018; Saadipour et al., Mol. Cell. Neurosci 99:103395, 2019; Yi., et al., EMBO J. 40: e104450, 2021), etc., and teaches that LM11A-31 salts, crystal forms thereof, and pharmaceutical compositions that include and/or are manufactured therefrom (e.g., from provided preparations thereof), may be useful in the treatment of various diseases, disorders or conditions associated with one or more such pathways. Indeed, the broad signaling activity of p75^{NTR} may render such provided salts, crystal forms, and/or pharmaceutical compositions uniquely useful and/or effective for treatment of one or more such conditions (e.g., Alzheimer's Disease).

The present disclosure, therefore, not only recognizes previously unknown problem(s) associated with the sulfate salt forms, but also provides solution(s) by providing salt and crystalline forms of a compound of Formula (I) that do not suffer from these same problem(s). For example, as illustrated in Example 2 herein, the sulfate salt form was found to deliquesce after one day at accelerated conditions (40° C./75% RH), whereas certain salts and/or crystalline forms provided herein exhibit stability for longer periods of time, including, for example, up to a week or longer. In some embodiments, provided salt and/or crystal forms exhibit stability (i.e., no substantial deliquescence) after a week at accelerated conditions (40° C./75% RH).

As further demonstrated herein, it is also surprising that additional salt and crystal forms of a compound of Formula (I) were identified. As illustrated in Example 2 in Table 26, at least 59 coformers were analyzed across at least 184 screening conditions to identify suitable salt forms that did not suffer the same problems associated with the previous sulfate salt form. Across all of these studies, only certain salt and crystalline solid forms exhibited sufficient stability (e.g., as assessed by stability of the salt or crystal form at accelerated conditions) for at least one week. These forms are described in various embodiments and examples herein.

In one aspect, the present disclosure provides a compound wherein the compound is a salt of a compound of Formula (I):

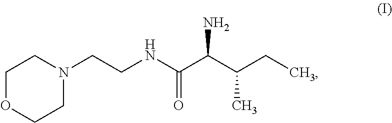 [SMILES: CC[C@H](C)[C@H](N)C(=O)NCCN1CCOCC1]

wherein the salt is an adipic salt or gentisic salt.

In another aspect, the present disclosure provides a crystalline form wherein the crystalline form is an adipic salt of a compound of Formula (I):

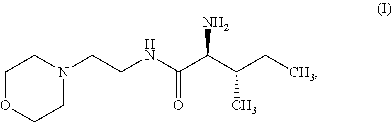 [SMILES: CC[C@H](C)[C@H](N)C(=O)NCCN1CCOCC1]

wherein the crystalline form exhibits an X-ray powder diffraction pattern comprising peaks at the following diffraction angles (2θ): 8.4±0.2, 15.4±0.2, 19.8±0.2, 21.0±0.2, and 22.6±0.2.

In another aspect, the present disclosure provides a crystalline form wherein the crystalline form is an orotic salt of a compound of Formula (I):

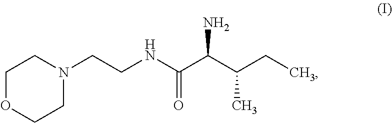 [SMILES: CC[C@H](C)[C@H](N)C(=O)NCCN1CCOCC1]

wherein the crystalline form exhibits an X-ray powder diffraction pattern comprising peaks at the following diffraction angles (2θ): 4.7±0.2, 14.4±0.2, 14.6±0.2, 15.9±0.2, and 27.3±0.2

In another aspect, the present disclosure provides a crystalline form wherein the crystalline form is a gentisic salt of a compound of Formula (I):

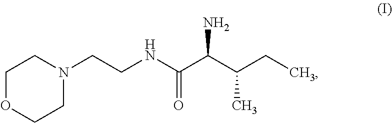 [SMILES: CC[C@H](C)[C@H](N)C(=O)NCCN1CCOCC1]

wherein the crystalline form exhibits an X-ray powder diffraction pattern comprising peaks at the following diffraction angles (2θ): 7.9±0.2, 17.3±0.2, 22.1±0.2, 23.1±0.2, and 24.5±0.2.

In another aspect, the present disclosure provides a crystalline form wherein the crystalline form is a tartaric salt of a compound of Formula (I):

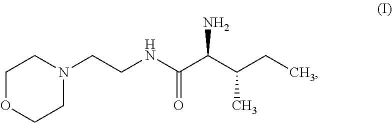 [SMILES: CC[C@H](C)[C@H](N)C(=O)NCCN1CCOCC1]

wherein the crystalline form exhibits an X-ray powder diffraction pattern comprising peaks at the following diffraction angles (2θ): 5.0±0.2, 10.9±0.2, 15.9±0.2, 16.0±0.2, and 20.8±0.2.

In another aspect, the present disclosure provides a crystalline form wherein the crystalline form is a 3-HXB salt of a compound of Formula (I):

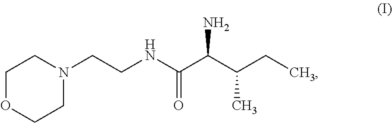 [SMILES: CC[C@H](C)[C@H](N)C(=O)NCCN1CCOCC1]

wherein the crystalline form exhibits an X-ray powder diffraction pattern comprising peaks at the following diffraction angles (2θ): 7.9±0.2, 17.4±0.2, 22.2±0.2, 23.4±0.2, and 24.7±0.2.

Also provided herein is a pharmaceutical composition comprising a provided salt of a compound of Formula (I), or a provided crystalline form, as disclosed herein, and a pharmaceutically acceptable excipient.

Provided herein, in part, is a method of treating Alzheimer's Disease in a subject in need thereof, comprising administering to the subject a therapeutically effective amount of the salt of a compound of Formula (I), or the crystalline form disclosed herein or the pharmaceutical composition disclosed herein.

Also provided herein is a method of producing a crystalline form described herein, the method comprising: combining a compound of Formula (I):

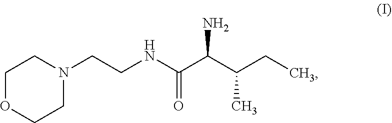 [SMILES: CC[C@H](C)[C@H](N)C(=O)NCCN1CCOCC1]

or a salt thereof, with a solvent, thereby forming a suspension or a solution thereof; optionally heating the suspension, thereby forming the solution; optionally cooling the solution; and forming a precipitate from the solution, thereby forming the crystalline form.

### BRIEF DESCRIPTION OF THE DRAWINGS

FIG. 1 depicts the ¹H NMR spectrum of the compound of Formula (I).

FIG. 2 depicts the XRPD of the compound of Formula (I) Form 1.

FIG. 3 depicts the XRPD comparison of the compound of Formula (I) Form 1 and the compound of Formula (I) reference standard.

FIG. 4 depicts the DSC and TGA of the compound of Formula (I) Form 1.

FIG. 5 depicts the XRPD comparison of ORO (orotic salt) Form A, ORO Form B, ORO Form C and a mixture of ORO Form A and ORO Form D.

FIG. 6 depicts the XRPD comparison of the MUC (mucic salt) Form A, MUC Form B and MUC Form C.

FIG. 7 depicts a GEN Form A microscopic image.

FIG. 8 depicts a GEN Form A microscopic image.

FIG. 9 depicts a GEN Form A microscopic image.

FIG. 10 depicts the XRPD of ADI (adipic salt) Form A prepared in EtOAc.

FIG. 11 depicts the ¹H NMR spectrum of ADI Form A prepared in EtOAc.

FIG. 12 depicts the DSC and TGA overlay of ADI Form A prepared in EtOAc.

FIG. 13 depicts the DVS analysis of ADI Form A prepared in EtOAc, weight vs relative humidity.

FIG. 14 depicts the DVS analysis of ADI Form A prepared in EtOAc, weight vs time.

FIG. 15 depicts the XRPD of ADI Form A prepared in THF.

FIG. 16 depicts the ¹H NMR spectrum of ADI Form A prepared in THF.

FIG. 17 depicts the DSC and TGA overlay of ADI Form A prepared in THF.

FIG. 18 depicts the DVS analysis of ADI Form A prepared in THF, weight vs relative humidity.

FIG. 19 depicts the DVS analysis of ADI Form A prepared in THF, weight vs time.

FIG. 20 depicts microscopy images of ADI Form A samples.

FIG. 21 depicts stability sample of GEN (gentisic salt) Form A and ORO Form A after 1 week.

FIG. 22 depicts the solution aspect for gentisate solubility experiments, from low pH (left) to high (right).

FIG. 23 depicts the solubility curve of GEN Form A in IPA.

FIG. 24 depicts the solubility curve of GEN Form A in EtOH.

FIG. 25 depicts the parity plot for GEN Form A solubility model in EtOAc-EtOH.

FIG. 26 depicts the solubility model for GEN Form A in EtOAc-EtOH.

FIG. 27 depicts the HPLC chromatogram of a sample of the compound of Formula (I) used to prepare various salts.

FIG. 28 depicts the XRPD overlay of non-stable crystalline forms.

FIG. 29 depicts the XRPD of ORO Form A.

FIG. 30 depicts the ¹H NMR spectrum of ORO Form A.

FIG. 31 depicts the DSC-TGA Overlay of ORO Form A.

FIG. 32 depicts the DVS analysis of ORO Form A, weight vs relative humidity.

FIG. 33 depicts the DVS analysis of ORO Form A, weight vs time.

FIG. 34 depicts the HPLC chromatogram of ORO Form A.

FIG. 35 depicts the XRPD of GEN Form A.

FIG. 36 depicts the ¹H NMR spectrum of GEN Form A.

FIG. 37 depicts the DSC and TGA overlay of GEN Form A.

FIG. 38 depicts the DVS analysis of GEN Form A, weight vs relative humidity.

FIG. 39 depicts the DVS analysis of GEN Form A, weight vs time.

FIG. 40 depicts the HPLC chromatogram of GEN Form A.

FIG. 41 depicts the XRPD of TAR (tartaric salt) Form A.

FIG. 42 depicts the ¹H NMR spectrum of TAR Form A.

FIG. 43 depicts the DSC and TGA overlay of TAR Form A.

FIG. 44 depicts the DVS analysis of TAR Form A, weight vs relative humidity.

FIG. 45 depicts the DVS analysis of TAR Form A, weight vs time.

FIG. 46 depicts the XRPD of 3-HXB (3-hydroxybenzoic salt) Form A.

FIG. 47 depicts the ¹H NMR of 3-HXB Form A.

FIG. 48 depicts the DSC and TGA overlay of 3-HXB Form A.

FIG. 49 depicts the DVS analysis of 3-HXB Form A, weight vs relative humidity.

FIG. 50 depicts the DVS analysis of 3-HXB Form A, weight vs time.

FIG. 51 depicts the solubility curve of ADI Form A in 2-MeTHF

FIG. 52 depicts the solubility curve of ADI Form A in EtOAc.

FIG. 53 depicts the XRPD of ORO Form B.

FIG. 54 depicts the XRPD of ORO Form C.

FIG. 55 depicts the XRPD of ORO Form D.

FIG. 56 depicts the XRPD of GEN Form B.

FIG. 57 depicts the XRPD of TAR Form C.

FIG. 58 depicts the XRPD of 2-MXB (2-methoxybenzoic salt) Form A.

FIG. 59 depicts the XRPD of 3-MXB (3-methoxybenzoic salt) Form A.

FIG. 60 depicts the XRPD of FUM (fumaric salt) Form A.

FIG. 61 depicts the XRPD of HIP Form A compared to other salt forms.

FIG. 62 depicts the XRPD of HIP (hippuric salt) Form B.

FIG. 63 depicts the XRPD of MUC Form A.

FIG. 64 depicts the XRPD of MUC Form B.

FIG. 65 depicts the XRPD of MUC Form C.

FIG. 66 depicts the XRPD of PRO (propionic salt) Form A.

FIG. 67 depicts the XRPD of the compound of Formula (I) Form 2.

FIG. 68 depicts the XRPD of the compound of Formula (I) Form 3.

FIG. 69 depicts the XRPD of the compound of Formula (I) Form 4.

FIG. 70 depicts the temperature and reflectance profile of ADI Form A.

FIG. 71 depicts a particle size analysis of ADI Form A.

### DETAILED DESCRIPTION

The present disclosure provides, in part, a salt or a crystalline form of a compound of Formula (I). A compound of Formula (I) is also known as LM11A-31. LM11A-31 is a mimetic of the loop 1 domain of NGF and the other neurotrophins (Massa et al., (2006) Neuropharmacology 110, 343-361). The current forms of LM11A-31 are not amenable to large-scale manufacturing, and thus, there is a need for developing a form of this compound that is suitable for drug development. The present disclosure identifies the source of one or more problem(s) with available salts of LM11A-31, including hygroscopicity and stability liabilities. In one aspect, salts and crystalline forms described herein exhibit desirable properties for drug development such as non-hygroscopicity and stability.

Certain features and other details of the disclosure will now be more particularly described. Before further description of the present disclosure, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

The terms “a,” “an,” and “the” refer to “one or more” when used in this application, including the claims. Thus, for example, reference to “a carrier” includes mixtures of one or more carriers, two or more carriers, and the like.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification and attached claims are approximations that can vary depending upon the desired properties sought to be obtained by the present application. Generally the term “about,” as used herein when referring to a measurable value such as an amount of weight, time, dose, etc. is meant to encompass in one example variations of ±20% or ±10%, in another example ±5%, in another example ±1%, and in yet another example ±0.10% from the specified amount, as such variations are appropriate to perform the disclosed method.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of, susceptibility to, severity of, stage of, etc the disease, disorder, or condition (e.g., across a relevant population).

“The compound of Formula (I) Form 1” and “Form 1” are used interchangeably herein. “The compound of Formula (I) Form 2” and “Form 2” are used interchangeably herein. “The compound of Formula (I) Form 3” and “Form 3” are used interchangeably herein. “The compound of Formula (I) Form 4” and “Form 4” are used interchangeably herein.

“Gentisate” and “gentisic” are used interchangeably herein. “Adipate” and “adipic” are used interchangeably herein. “Orotate” and “orotic” are used interchangeably herein. “Tartrate,” “tartarate,” and “tartaric” are used interchangeably herein. “3-hydroxybenzoate”, “3-hydroxybenzoic”, and “3-HXB” are used interchangeably herein. “2-methoxybenzoate”, “2-methoxybenzoic”, and “2-MXB” are used interchangeably herein. “3-methoxybenzoate”, “3-methoxybenzoic”, and “3-MXB” are used interchangeably herein. “Fumarate” and “fumaric” are used interchangeably herein. “Hippurate” and “hippuric” are used interchangeably herein. “Mucate” and “mucic” are used interchangeably herein. “Propionate” and “propionic” are used interchangeably herein. “BHS” and “sulfate” are used interchangeably herein.

“Disease,” “disorder,” and “condition” are used interchangeably herein.

As used herein, the “effective amount” of a compound or a crystalline form thereof refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

The terms “excipient,” “carrier,” and “vehicle” are used interchangeably throughout this application and denote a substance with which a compound of the present disclosure is administered.

The term “substantially the same” as used herein means an analytical result, e.g., X-ray powder diffraction (XRPD) pattern, dynamic vapor sorption curve (DVS), differential scanning calorimetry (DSC) curve, thermogravimetric analysis (TGA) curve, ¹H nuclear magnetic resonance (NMR) spectrum, particle size distribution (PSD) curve, which resembles the reference spectrum to a great degree in peak, signal, chemical shift, curve, point, etc. and locations and intensities of peaks, signals, chemical shift, etc.

As used herein, and unless otherwise specified, a “therapeutically effective amount” of a compound or a crystalline form thereof is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. Those skilled in the art will appreciate that a “therapeutically effective amount” may be an amount that is effective when administered as part of a dosing regimen that has been determined to be, statistically, reasonably effective when administered to a relevant population. Thus, a “therapeutically effective amount” is more than a de minimis amount, but need not be an amount that would be effective in a single dose.

The term “treatment” as used herein covers any treatment of a disease and/or condition in an animal or mammal, particularly a human, and includes: (i) preventing a disease, disorder and/or condition and/or symptoms from occurring in a person which can be predisposed to the disease, disorder and/or condition, or at risk for being exposed to an agent that can cause the disease, disorder, and/or condition and/or symptoms; but, has not yet been diagnosed as having it; (ii) inhibiting the disease, disorder and/or condition, and/or symptoms i.e., arresting its development; (iii) slowing progress of the disease, disorder and/or condition, and/or symptoms; and (iv) relieving the disease, disorder and/or condition, and/or symptoms i.e., causing regression of the disease, disorder and/or condition; (v) augmenting a mechanism, such as modulating p75^{NTR} signaling, that can lead to reduced symptoms and improved function.

### Salts of a Compound of Formula (I)

The disclosure provides in part, a compound wherein the compound is a salt of a compound of Formula (I):

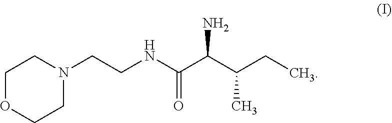 [SMILES: CC[C@H](C)[C@H](N)C(=O)NCCN1CCOCC1]

In some embodiments, the salt is an adipic salt or gentisic salt.

In some embodiments, the salt is an adipic salt.

In some embodiments, the adipic salt is:

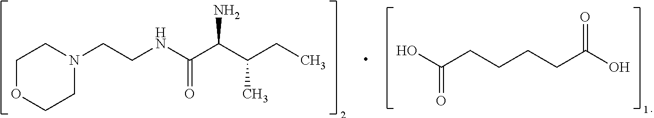

In some embodiments, the adipic salt is:

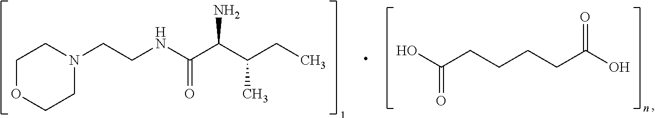

wherein n is about 0.5 to about 2. In some embodiments, n is 0.5.

In some embodiments, the salt is a gentisic salt.

In some embodiments, the gentisic salt is:

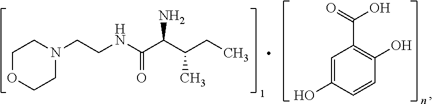

wherein n is about 0.5 to about 2. In some embodiments, n is 1.

In some embodiments, the salt is an orotic salt.

In some embodiments, the orotic salt is:

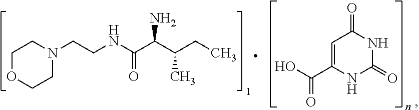

wherein n is about 0.5 to about 2. In some embodiments, n is 0.7.

In some embodiments, the salt is a tartaric salt.

In some embodiments, the tartaric salt is:

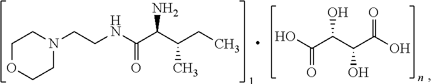

wherein n is about 0.5 to about 2. In some embodiments, n is 1.

In some embodiments, the tartaric salt is:

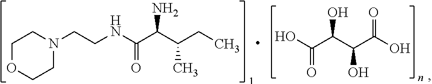

wherein n is about 0.5 to about 2. In some embodiments, n is 1.

In some embodiments, the tartaric salt is:

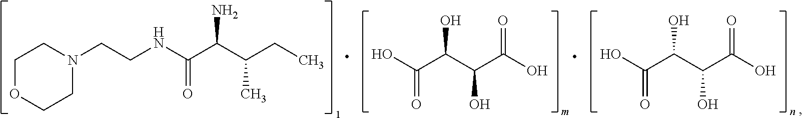

Wherein m and n are each independently about 0.5 to about 2. In some embodiments, n is 1 and m is 1.

In some embodiments, the tartaric salt is:

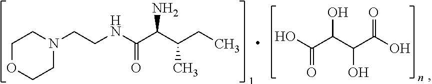

wherein n is about 0.5 to about 2. In some embodiments, n is 1.

In some embodiments, the tartaric salt is:

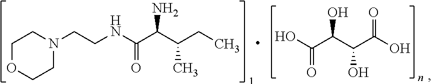

wherein n is about 0.5 to about 2. In some embodiments, n is 1.

In some embodiments, the salt is a 2-methoxybenzoic (2-MXB) salt. In some embodiments, the 2-methoxybenzoic salt is:

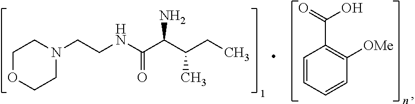

wherein n is about 0.5 to about 2. In some embodiments, n is 1.

In some embodiments, the salt is a 3-hydroxybenzoic (3-HXB) salt. In some embodiments, the 3-HXB salt is:

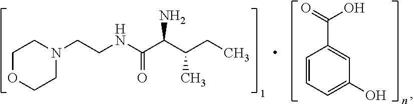

wherein n is about 0.5 to about 2. In some embodiments, n is 1.

In some embodiments, the salt is a 3-methoxybenzoic salt. In some embodiments, the 3-methoxybenzoic (3-MXB) salt is:

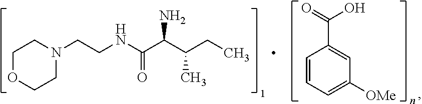

wherein n is about 0.5 to about 2. In some embodiments, n is 1.

In some embodiments, the salt is a fumaric salt. In some embodiments, the fumaric salt is:

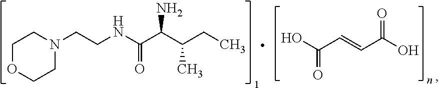

wherein n is about 0.5 to about 2. In some embodiments, n is 1.

In some embodiments, the salt is a hippuric salt. In some embodiments, the hippuric salt is:

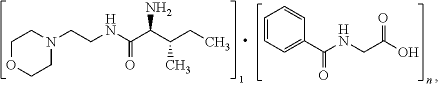

wherein n is about 0.5 to about 2. In some embodiments, n is 1.

In some embodiments, the salt is a mucic salt. In some embodiments, the mucic salt is:

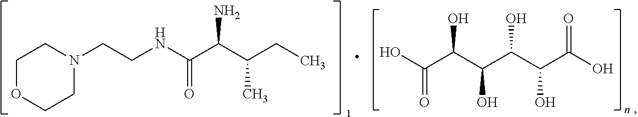

wherein n is about 0.5 to about 2. In some embodiments, n is 1.

In some embodiments, the salt is a propionic salt. In some embodiments, the propionic salt is:

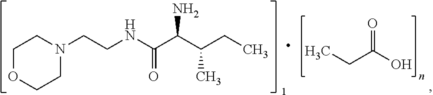

wherein n is about 0.5 to about 2. In some embodiments, n is 1.

In some embodiments, a compound of the present disclosure is provided and/or utilized in a solid form, for example in a crystal form (e.g., hydrate, solvate and/or anhydrous crystal form).

### ADI Form A

In one aspect, an adipic salt of a compound of Formula (I):

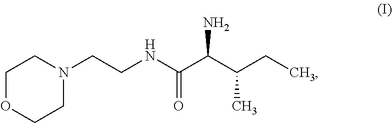 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 8.4±0.2, 19.8±0.2, and 21.0±0.2.

In some embodiments, such a crystalline form exhibits an X-ray powder diffraction pattern comprising peaks at the following diffraction angles (2θ): 8.4±0.2, 15.4±0.2, 19.8±0.2, 21.0±0.2, and 22.6±0.2.

In some embodiments, such a crystalline form exhibits an X-ray powder diffraction pattern comprising peaks at the following diffraction angles (2θ): 8.4±0.2, 13.2±0.2, 15.4±0.2, 18.3±0.2, 18.9±0.2, 19.8±0.2, 21.0±0.2, 21.4±0.2, 22.6±0.2, and 24.4±0.2.

In some embodiments, the crystalline form exhibits an X-ray powder diffraction pattern comprising peaks selected from Table 1.

TABLE 1
XRPD Peak List of ADI Form A

| | Position (°2- | Intensity | |
|---|---|---|---|
| Peak | theta) | (Counts) | Rel. Int. (%) |
| 1 | 5.2 | 4133.0 | 17.9 |
| 2 | 8.4 | 23033.0 | 100.0 |
| 3 | 9.6 | 1906.0 | 8.3 |
| 4 | 10.5 | 2333.0 | 10.1 |
| 5 | 13.2 | 9590.0 | 41.6 |
| 6 | 15.4 | 14772.0 | 64.1 |
| 7 | 16.3 | 3142.0 | 13.6 |
| 8 | 16.9 | 4659.0 | 20.2 |
| 9 | 17.7 | 2502.0 | 10.9 |
| 10 | 17.9 | 4350.0 | 18.9 |
| 11 | 18.3 | 7107.0 | 30.9 |
| 12 | 18.9 | 7899.0 | 34.3 |
| 13 | 19.5 | 3750.0 | 16.3 |
| 14 | 19.8 | 17982.0 | 78.1 |
| 15 | 20.0 | 8076.0 | 35.1 |
| 16 | 20.5 | 4796.0 | 20.8 |
| 17 | 20.8 | 4293.0 | 18.6 |
| 18 | 21.0 | 14823.0 | 64.4 |
| 19 | 21.4 | 6063.0 | 26.3 |
| 20 | 21.7 | 1879.0 | 8.2 |
| 21 | 22.0 | 1471.0 | 6.4 |
| 22 | 22.6 | 9945.0 | 43.2 |
| 23 | 22.9 | 1349.0 | 5.9 |
| 24 | 23.8 | 2566.0 | 11.1 |
| 25 | 24.4 | 5028.0 | 21.8 |
| 26 | 24.8 | 3060.0 | 13.3 |
| 27 | 25.5 | 2883.0 | 12.5 |
| 28 | 26.4 | 1553.0 | 6.7 |
| 29 | 26.6 | 1437.0 | 6.2 |
| 30 | 27.6 | 1584.0 | 6.9 |
| 31 | 29.0 | 1622.0 | 7.0 |
| 32 | 30.2 | 1256.0 | 5.5 |
| 33 | 30.6 | 2282.0 | 9.9 |
| 34 | 32.3 | 1158.0 | 5.0 |
| 35 | 32.6 | 1053.0 | 4.6 |
| 36 | 37.2 | 2147.0 | 9.3 |
| 37 | 37.6 | 1675.0 | 7.3 |
| 38 | 38.0 | 1185.0 | 5.1 |
| 39 | 38.2 | 1503.0 | 6.5 |
| 40 | 38.5 | 1158.0 | 5.0 |

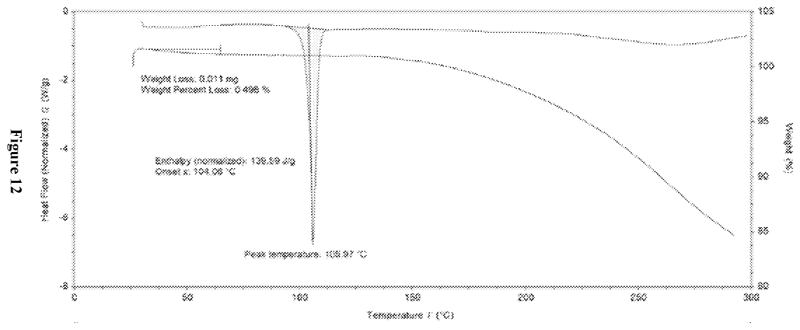

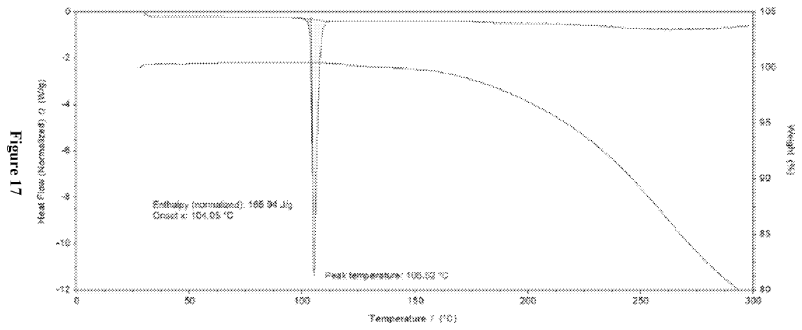

In some embodiments, the crystalline form exhibits an X-ray powder diffraction pattern substantially the same as depicted in FIG. 10 or FIG. 15.

In some embodiments, the crystalline form has a melting point onset as determined by differential scanning calorimetry at about 104° C.

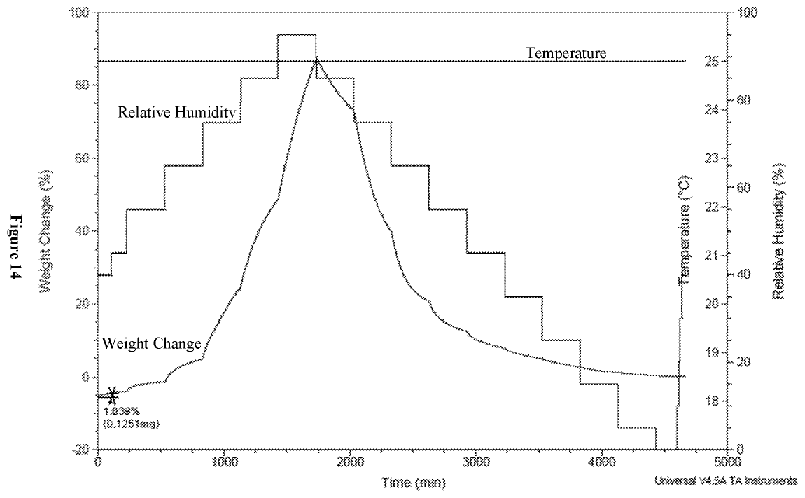

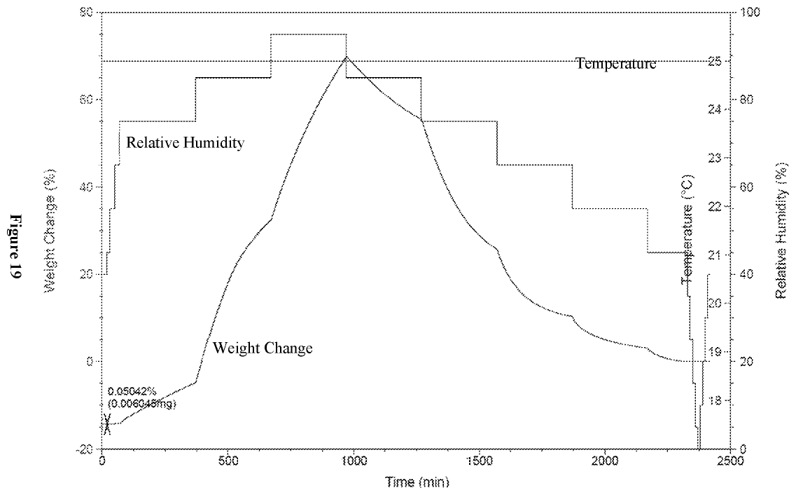

In some embodiments, the crystalline form has a differential scanning calorimetry curve substantially the same as shown in FIG. 12 or FIG. 17.

In some embodiments, the crystalline form is represented by:

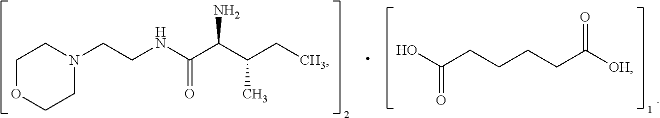

In some embodiments, the crystalline form described herein is prepared by a method comprising: combining a compound of Formula (I):

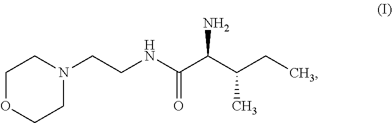 [SMILES: CC[C@H](C)[C@H](N)C(=O)NCCN1CCOCC1]

or a salt thereof, with a solvent, thereby forming a suspension or a solution thereof; optionally heating the suspension, thereby forming the solution; optionally cooling the solution; and forming a precipitate from the solution, thereby forming the crystalline form.

In some embodiments, the crystalline form provided herein forms particles. In some embodiments, 90% of the particles are smaller than about 600 μm to about 640 μm. In some embodiments, 90% of the particles are smaller than about 605 μm to about 635 μm. In some embodiments, 90% of the particles are smaller than about 610 μm to about 630 μm. In some embodiments, 90% of the particles are smaller than about 615 μm to about 625 μm. In some embodiments, 90% of the particles are smaller than about 620 μm to about 625 μm. In some embodiments, 90% of the particles are smaller than about 621 μm to about 625 μm. In some embodiments, 90% of the particles are smaller than about 622 μm to about 625 μm. In some embodiments, 90% of the particles are smaller than about 623 μm to about 625 μm. In some embodiments, 90% of the particles are smaller than about 624 μm.

In some embodiments, 50% of the particles are smaller than about 20 μm to about 40 μm. In some embodiments, 50% of the particles are smaller than about 25 μm to about 35 μm. In some embodiments, 50% of the particles are smaller than about 29 μm to about 31 μm. In some embodiments, 50% of the particles are smaller than about 31 μm. In some embodiments, 50% of the particles are smaller than about 30.7 μm.

In some embodiments, 10% of the particles are smaller than about 4 μm to about 5 μm. In some embodiments, 10% of the particles are smaller than about 4.1 μm to about 4.9 μm. In some embodiments, 10% of the particles are smaller than about 4.2 μm to about 4.8 μm. In some embodiments, 10% of the particles are smaller than about 4.3 μm to about 4.7 μm. In some embodiments, 10% of the particles are smaller than about 4.4 μm to about 4.6 μm. In some embodiments, 10% of the particles are smaller than about 4.4 μm to about 4.5 μm. In some embodiments, 10% of the particles are smaller than about 4.43 μm.

In some embodiments, the particle size is as measured by a laser diffraction instrument such as Malvern Mastersizer 3000.

### ORO Form A

In one aspect, an orotic salt of a compound of Formula (I):

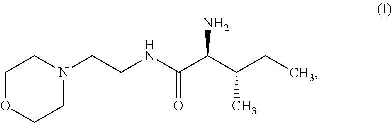 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 4.7±0.2, 14.4±0.2, and 27.3±0.2.

In some embodiments, such a crystalline form exhibits an X-ray powder diffraction pattern comprising peaks at the following diffraction angles (2θ): 4.7±0.2, 14.4±0.2, 14.6±0.2, 15.9±0.2, and 27.3±0.2.

In some embodiments, such a crystalline form exhibits an X-ray powder diffraction pattern comprising peaks at the following diffraction angles (2θ): 4.7±0.2, 14.4±0.2, 14.6±0.2, 15.9±0.2, 16.1±0.2, 18.8±0.2, 20.6±0.2, 22.2±0.2, 26.9±0.2, and 27.3±0.2.

In some embodiments, the crystalline form exhibits an X-ray powder diffraction pattern comprising peaks selected from Table 2.

TABLE 2
XRPD Peak List of ORO Form A

| | Position (°2- | Intensity | |
|---|---|---|---|
| Peak | theta) | (Counts) | Rel. Int. (%) |
| 1 | 4.7 | 49322.0 | 100.0 |
| 2 | 9.5 | 3395.0 | 6.9 |
| 3 | 11.0 | 3900.0 | 7.9 |
| 4 | 11.6 | 1779.0 | 3.6 |
| 5 | 14.4 | 8199.0 | 16.6 |
| 6 | 14.6 | 7671.0 | 15.6 |
| 7 | 15.9 | 7720.0 | 15.7 |
| 8 | 16.1 | 6701.0 | 13.6 |
| 9 | 17.1 | 2971.0 | 6.0 |
| 10 | 18.1 | 2290.0 | 4.6 |
| 11 | 18.4 | 2697.0 | 5.5 |
| 12 | 18.8 | 5192.0 | 10.5 |
| 13 | 19.0 | 2396.0 | 4.9 |
| 14 | 20.6 | 5076.0 | 10.3 |
| 15 | 20.9 | 2604.0 | 5.3 |
| 16 | 21.1 | 3308.0 | 6.7 |
| 17 | 21.4 | 2765.0 | 5.6 |
| 18 | 22.2 | 7127.0 | 14.4 |
| 19 | 23.2 | 2781.0 | 5.6 |
| 20 | 24.2 | 1490.0 | 3.0 |
| 21 | 25.4 | 2495.0 | 5.1 |
| 22 | 25.6 | 1527.0 | 3.1 |
| 23 | 25.9 | 1689.0 | 3.4 |
| 24 | 26.9 | 6544.0 | 13.3 |
| 25 | 27.3 | 9394.0 | 19.0 |
| 26 | 28.6 | 3822.0 | 7.7 |
| 27 | 30.5 | 1553.0 | 3.1 |
| 28 | 30.7 | 1293.0 | 2.6 |
| 29 | 31.2 | 1219.0 | 2.5 |
| 30 | 35.7 | 1232.0 | 2.5 |

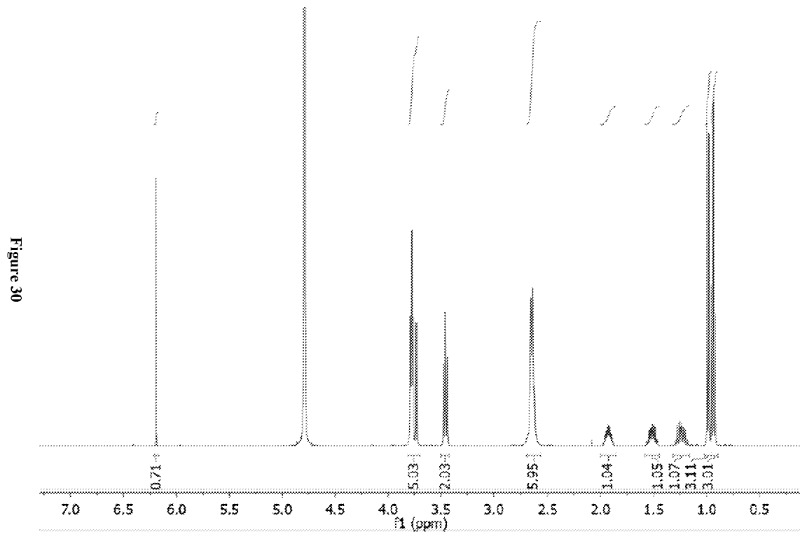

In some embodiments, the crystalline form exhibits an X-ray powder diffraction pattern substantially the same as depicted in FIG. 29.

In some embodiments, the crystalline form has a melting point onset as determined by differential scanning calorimetry at about 191° C.

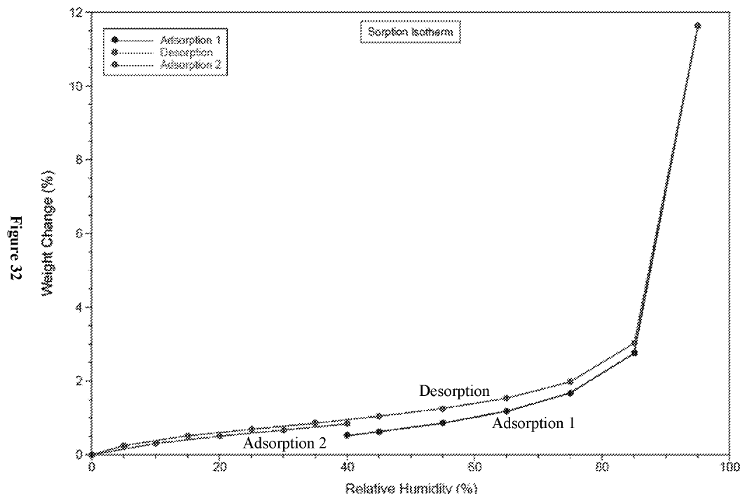

In some embodiments, the crystalline form has a differential scanning calorimetry curve substantially the same as shown in FIG. 31.

### GEN Form A

In one aspect, a gentisic salt of a compound of Formula (I):

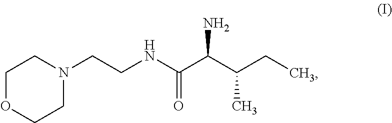 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 7.9±0.2, 17.3±0.2, and 23.1±0.2.

In some embodiments, such a crystalline form exhibits an X-ray powder diffraction pattern comprising peaks at the following diffraction angles (2θ): 7.9±0.2, 17.3±0.2, 22.1±0.2, 23.1±0.2, and 24.5±0.2.

In some embodiments, such a crystalline form exhibits an X-ray powder diffraction pattern comprising peaks at the following diffraction angles (2θ): 7.9±0.2, 11.0±0.2, 15.8±0.2, 17.3±0.2, 17.6±0.2, 20.6±0.2, 22.1±0.2, 23.1±0.2, 24.5±0.2, and 26.1±0.2.

In some embodiments, the crystalline form exhibits an X-ray powder diffraction pattern comprising peaks selected from Table 3.

TABLE 3
XRPD Peak List of GEN Form A

| | Position (°2- | Intensity | |
|---|---|---|---|
| Peak | theta) | (Counts) | Rel. Int. (%) |
| 1 | 7.9 | 57314.0 | 100.0 |
| 2 | 11.0 | 10734.0 | 18.7 |
| 3 | 15.8 | 15284.0 | 26.7 |
| 4 | 17.3 | 44989.0 | 78.5 |
| 5 | 17.6 | 27128.0 | 47.3 |
| 6 | 20.6 | 19929.0 | 34.8 |
| 7 | 21.0 | 5427.0 | 9.5 |
| 8 | 22.1 | 32651.0 | 57.0 |
| 9 | 22.4 | 5356.0 | 9.3 |
| 10 | 23.1 | 44381.0 | 77.4 |
| 11 | 23.9 | 2932.0 | 5.1 |
| 12 | 24.5 | 33595.0 | 58.6 |
| 13 | 25.1 | 1852.0 | 3.2 |
| 14 | 26.1 | 12022.0 | 21.0 |
| 15 | 27.0 | 8379.0 | 14.6 |
| 16 | 28.2 | 6554.0 | 11.4 |
| 17 | 28.8 | 4259.0 | 7.4 |
| 18 | 29.8 | 4469.0 | 7.8 |
| 19 | 31.5 | 3069.0 | 5.4 |
| 20 | 31.8 | 1677.0 | 2.9 |
| 21 | 32.1 | 1885.0 | 3.3 |
| 22 | 32.8 | 2057.0 | 3.6 |
| 23 | 34.1 | 2021.0 | 3.5 |
| 24 | 35.0 | 3160.0 | 5.5 |
| 25 | 35.8 | 2468.0 | 4.3 |
| 26 | 36.8 | 2513.0 | 4.4 |
| 27 | 37.8 | 3993.0 | 7.0 |
| 28 | 38.4 | 1925.0 | 3.4 |

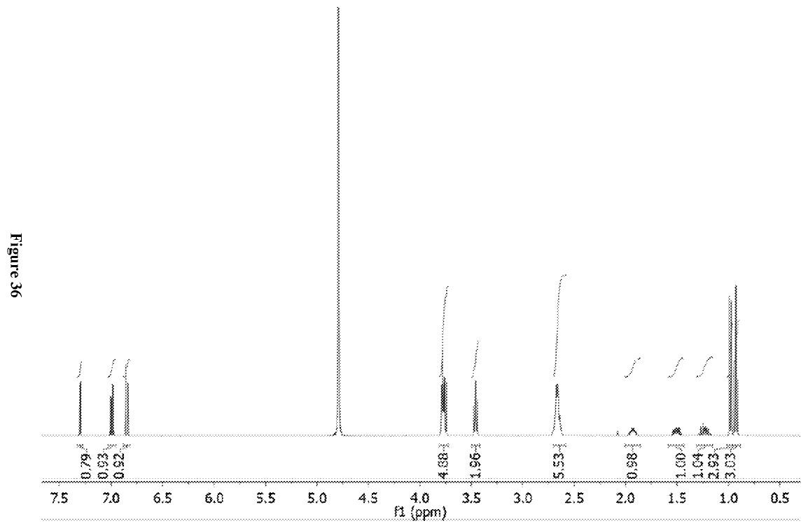

In some embodiments, the crystalline form exhibits an X-ray powder diffraction pattern substantially the same as depicted in FIG. 35.

In some embodiments, the crystalline form has a melting point onset as determined by differential scanning calorimetry at about 167° C.

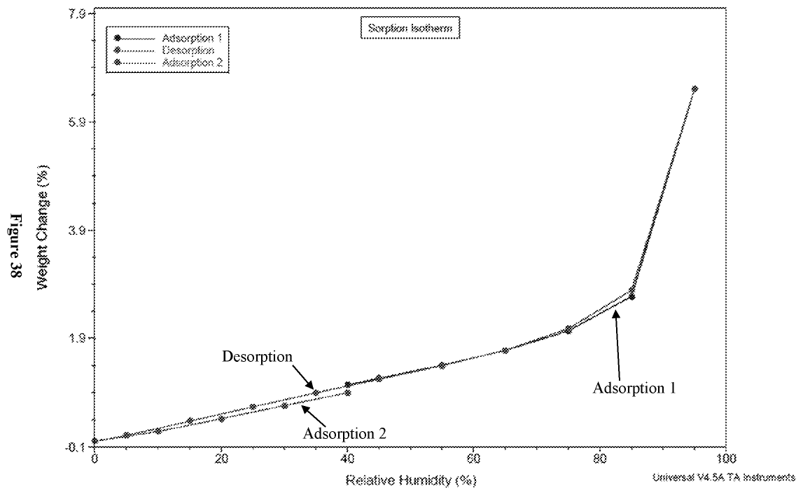

In some embodiments, the crystalline form has a differential scanning calorimetry curve substantially the same as shown in FIG. 37.

### TAR Form A

In one aspect, a tartaric salt of a compound of Formula (I):

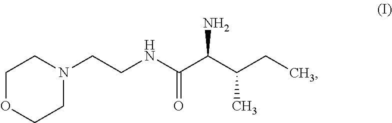 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 5.0±0.2, 10.9±0.2, and 16.0±0.2.

In some embodiments, such a crystalline form exhibits an X-ray powder diffraction pattern comprising peaks at the following diffraction angles (2θ): 5.0±0.2, 10.9±0.2, 15.9±0.2, 16.0±0.2, and 20.8±0.2.

In some embodiments, such a crystalline form exhibits an X-ray powder diffraction pattern comprising peaks at the following diffraction angles (2θ): 5.0±0.2, 10.9±0.2, 13.1±0.2, 15.9±0.2, 16.0±0.2, 16.2±0.2, 17.2±0.2, 17.7±0.2, 20.5±0.2, and 20.8±0.2.

In some embodiments, the crystalline form exhibits an X-ray powder diffraction pattern comprising some peaks selected from Table 4.

TABLE 4
XRPD Peak List of TAR Form A

| | Position (°2- | Intensity | |
|---|---|---|---|
| Peak | theta) | (Counts) | Rel. Int. (%) |
| 1 | 5.0 | 9075.0 | 100.0 |
| 2 | 10.1 | 1573.0 | 17.3 |
| 3 | 10.7 | 1829.0 | 20.2 |
| 4 | 10.9 | 3509.0 | 38.7 |
| 5 | 11.8 | 1556.0 | 17.1 |
| 6 | 13.1 | 2171.0 | 23.9 |
| 7 | 14.2 | 1510.0 | 16.6 |
| 8 | 15.9 | 3251.0 | 35.8 |
| 9 | 16.0 | 3428.0 | 37.8 |
| 10 | 16.2 | 2611.0 | 28.8 |
| 11 | 16.8 | 2232.0 | 24.6 |
| 12 | 17.2 | 1633.0 | 18.0 |
| 13 | 17.7 | 1967.0 | 21.7 |
| 14 | 18.6 | 1514.0 | 16.7 |
| 15 | 19.0 | 1889.0 | 20.8 |
| 16 | 19.2 | 1288.0 | 14.2 |
| 17 | 19.7 | 1298.0 | 14.3 |
| 18 | 20.3 | 1563.0 | 17.2 |
| 19 | 20.5 | 2100.0 | 23.1 |
| 20 | 20.8 | 3117.0 | 34.3 |
| 21 | 21.6 | 1679.0 | 18.5 |
| 22 | 22.0 | 1171.0 | 12.9 |
| 23 | 22.1 | 1149.0 | 12.7 |
| 24 | 22.7 | 1206.0 | 13.3 |
| 25 | 23.7 | 1362.0 | 15.0 |
| 26 | 24.0 | 1183.0 | 13.0 |
| 27 | 24.4 | 1023.0 | 11.3 |
| 28 | 25.5 | 1088.0 | 12.0 |
| 29 | 28.0 | 849.0 | 9.4 |

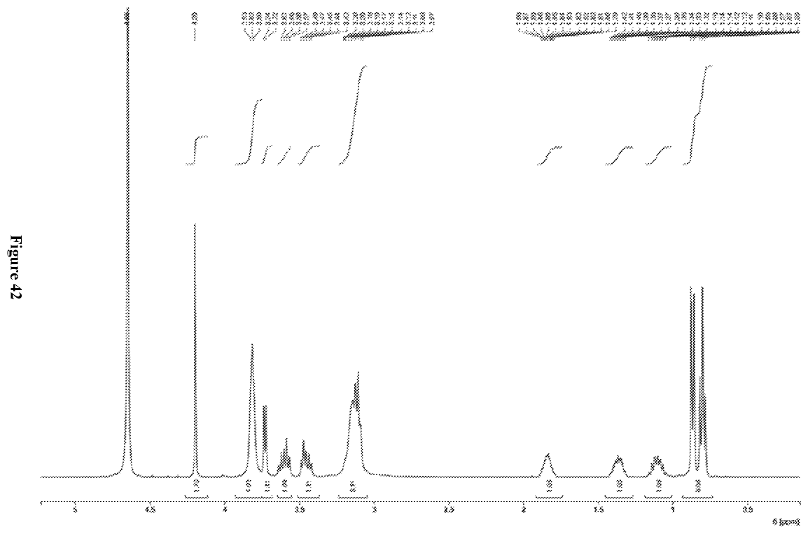

In some embodiments, the crystalline form exhibits an X-ray powder diffraction pattern substantially the same as depicted in FIG. 41.

In some embodiments, the crystalline form has a melting point onset as determined by differential scanning calorimetry at about 105° C.

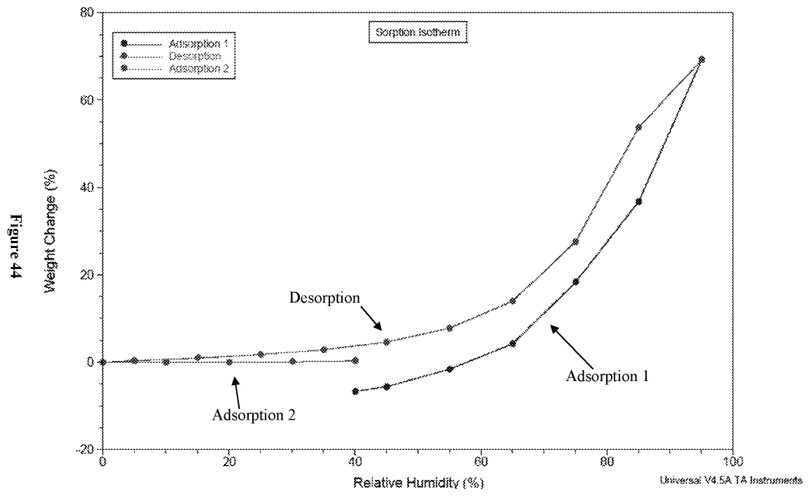

In some embodiments, the crystalline form has a differential scanning calorimetry curve substantially the same as shown in FIG. 43.

In one aspect, a 3-HXB salt of a compound of Formula (I):

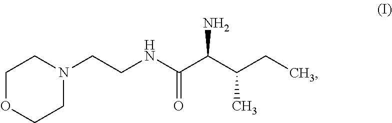 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 7.9±0.2, 17.4±0.2, and 23.4±0.2.

In some embodiments, such a crystalline form exhibits an X-ray powder diffraction pattern comprising peaks at the following diffraction angles (2θ): 7.9±0.2, 17.4±0.2, 22.2±0.2, 23.4±0.2, and 24.7±0.2.

In some embodiments, such a crystalline form exhibits an X-ray powder diffraction pattern comprising peaks at the following diffraction angles (2θ): 7.9±0.2, 11.1±0.2, 15.9±0.2, 17.4±0.2, 17.7±0.2, 20.8±0.2, 22.2±0.2, 23.4±0.2, 24.7±0.2, and 26.2±0.2.

In some embodiments, the crystalline form exhibits an X-ray powder diffraction pattern comprising some peaks selected from Table 5.

TABLE 5
XRPD Peak List of 3-HXB Form A

| | Position (°2- | Intensity | |
|---|---|---|---|
| Peak | theta) | (Counts) | Rel. Int. (%) |
| 1 | 7.9 | 27646.0 | 100.0 |
| 2 | 11.1 | 5403.0 | 19.5 |
| 3 | 15.9 | 8149.0 | 29.5 |
| 4 | 17.4 | 17674.0 | 63.9 |
| 5 | 17.7 | 8214.0 | 29.7 |
| 6 | 20.8 | 8659.0 | 31.3 |
| 7 | 21.0 | 3652.0 | 13.2 |
| 8 | 22.2 | 10172.0 | 36.8 |
| 9 | 22.4 | 2739.0 | 9.9 |
| 10 | 23.4 | 12523.0 | 45.3 |
| 11 | 23.9 | 1787.0 | 5.5 |
| 12 | 24.7 | 11694.0 | 42.3 |
| 13 | 26.2 | 4771.0 | 17.3 |
| 14 | 27.2 | 2683.0 | 9.7 |
| 15 | 28.3 | 2586.0 | 9.4 |
| 16 | 28.8 | 1561.0 | 5.6 |
| 17 | 29.9 | 1576.0 | 5.7 |
| 18 | 31.6 | 1070.0 | 3.9 |
| 19 | 32.9 | 1228.0 | 4.4 |
| 20 | 35.9 | 1181.0 | 4.3 |
| 21 | 36.9 | 1188.0 | 4.3 |
| 22 | 38.0 | 1614.0 | 5.8 |

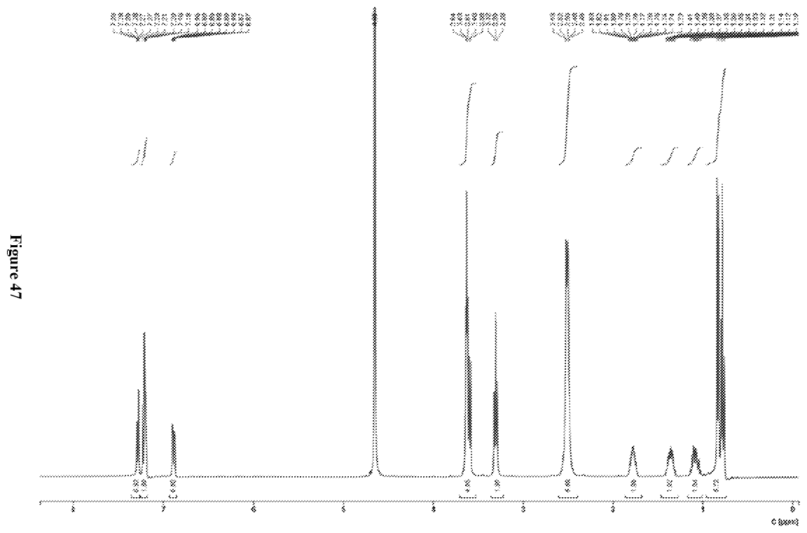

In some embodiments, the crystalline form exhibits an X-ray powder diffraction pattern substantially the same as depicted in FIG. 46.

In some embodiments, the crystalline form has a melting point onset as determined by differential scanning calorimetry at about 134° C.

In some embodiments, the crystalline form has a differential scanning calorimetry curve substantially the same as shown in FIG. 48.

### ORO Form B

In one aspect, an orotic salt of a compound of Formula (I):

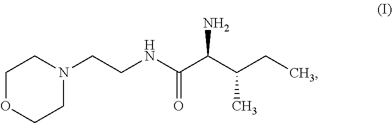 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 5.3±0.2, 15.7±0.2, and 20.7±0.2.

In some embodiments, such a crystalline form exhibits an X-ray powder diffraction pattern comprising peaks at the following diffraction angles (2θ): 5.3±0.2, 15.7±0.2, 16.2±0.2, 16.6±0.2, and 20.7±0.2.

In some embodiments, such a crystalline form exhibits an X-ray powder diffraction pattern comprising peaks at the following diffraction angles (2θ): 5.3±0.2, 10.6±0.2, 14.0±0.2, 15.7±0.2, 16.2±0.2, 16.6±0.2, 17.6±0.2, 19.9±0.2, 20.7±0.2, and 23.7±0.2.

In some embodiments, the crystalline form exhibits an X-ray powder diffraction pattern comprising some peaks selected from Table 6.

TABLE 6
XRPD Peak List of ORO Form B

| | Position (°2- | Intensity | |
|---|---|---|---|
| Peak | theta) | (Counts) | Rel. Int. (%) |
| 1 | 5.3 | 1970.0 | 100.0 |
| 2 | 9.9 | 523.0 | 26.5 |
| 3 | 10.6 | 920.0 | 46.7 |
| 4 | 12.8 | 467.0 | 23.7 |
| 5 | 14.0 | 795.0 | 40.4 |
| 6 | 15.1 | 478.0 | 24.3 |
| 7 | 15.7 | 1446.0 | 73.4 |
| 8 | 16.2 | 984.0 | 49.9 |
| 9 | 16.6 | 928.0 | 47.1 |
| 10 | 17.6 | 756.0 | 38.4 |
| 11 | 19.9 | 717.0 | 36.4 |
| 12 | 20.7 | 1005.0 | 51.0 |
| 13 | 21.4 | 561.0 | 28.5 |
| 14 | 22.5 | 463.0 | 23.5 |
| 15 | 23.5 | 613.0 | 31.1 |
| 16 | 23.7 | 695.0 | 35.3 |
| 17 | 25.1 | 380.0 | 19.3 |
| 18 | 26.0 | 434.0 | 22.0 |
| 19 | 36.5 | 242.0 | 12.3 |

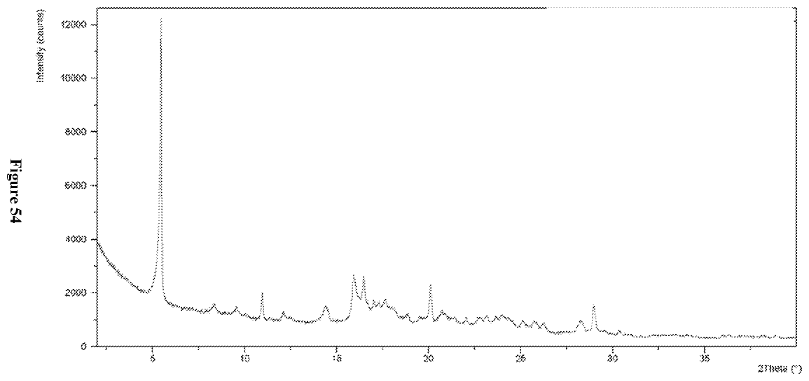

In some embodiments, the crystalline form exhibits an X-ray powder diffraction pattern substantially the same as depicted in FIG. 53.

### ORO Form C

In one aspect, an orotic salt of a compound of Formula (I):

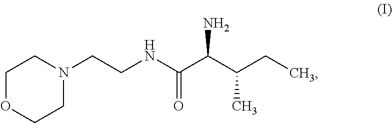 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 5.4±0.2, 15.8±0.2, and 16.3±0.2.

In some embodiments, such a crystalline form exhibits an X-ray powder diffraction pattern comprising peaks at the following diffraction angles (2θ): 5.4±0.2, 10.8±0.2, 15.8±0.2, 16.3±0.2, and 19.9±0.2.

In some embodiments, such a crystalline form exhibits an X-ray powder diffraction pattern comprising peaks at the following diffraction angles (2θ): 5.4±0.2, 10.8±0.2, 15.8±0.2, 16.1±0.2, 16.3±0.2, 16.5±0.2, 16.8±0.2, 17.2±0.2, 17.5±0.2, and 19.9±0.2.

In some embodiments, the crystalline form exhibits an X-ray powder diffraction pattern comprising some peaks selected from Table 7.

TABLE 7
XRPD Peak List of ORO Form C

| | Position (°2- | Intensity | |
|---|---|---|---|
| Peak | theta) | (Counts) | Rel. Int. (%) |
| 1 | 5.4 | 12234.0 | 100.0 |
| 2 | 8.3 | 1621.0 | 13.2 |
| 3 | 9.4 | 1507.0 | 12.3 |
| 4 | 10.8 | 2032.0 | 16.6 |
| 5 | 12.0 | 1325.0 | 10.8 |
| 6 | 14.3 | 1581.0 | 12.9 |
| 7 | 15.8 | 2726.0 | 22.3 |
| 8 | 16.1 | 1860.0 | 15.2 |
| 9 | 16.3 | 2616.0 | 21.4 |
| 10 | 16.5 | 1696.0 | 13.9 |
| 11 | 16.8 | 1750.0 | 14.3 |
| 12 | 17.0 | 1584.0 | 12.9 |
| 13 | 17.2 | 1696.0 | 13.9 |
| 14 | 17.5 | 1795.0 | 14.7 |
| 15 | 17.8 | 1496.0 | 12.2 |
| 16 | 18.0 | 1426.0 | 11.7 |
| 17 | 19.9 | 2343.0 | 19.2 |
| 18 | 20.5 | 1374.0 | 11.2 |
| 19 | 22.9 | 1209.0 | 9.9 |
| 20 | 23.4 | 1152.0 | 9.4 |
| 21 | 23.8 | 1179.0 | 9.6 |
| 22 | 24.1 | 1096.0 | 9.0 |
| 23 | 28.0 | 986.0 | 8.1 |
| 24 | 28.7 | 1547.0 | 12.6 |

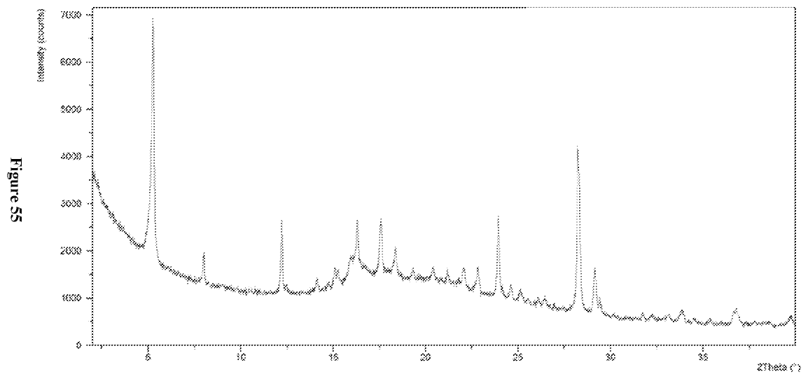

In some embodiments, the crystalline form exhibits an X-ray powder diffraction pattern substantially the same as depicted in FIG. 54.

### ORO Form D

In one aspect, an orotic salt of a compound of Formula (I):

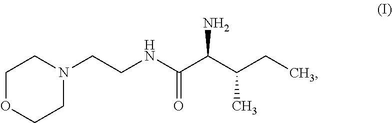 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 5.2±0.2, 23.7±0.2, and 27.9±0.2.

In some embodiments, such a crystalline form exhibits an X-ray powder diffraction pattern comprising peaks at the following diffraction angles (2θ): 5.2±0.2, 12.1±0.2, 17.4±0.2, 23.7±0.2, and 27.9±0.2.

In some embodiments, such a crystalline form exhibits an X-ray powder diffraction pattern comprising peaks at the following diffraction angles (2θ): 5.2±0.2, 7.9±0.2, 12.1±0.2, 16.1±0.2, 17.4±0.2, 18.2±0.2, 19.1±0.2, 20.2±0.2, 23.7±0.2, and 27.9±0.2.

In some embodiments, the crystalline form exhibits an X-ray powder diffraction pattern comprising some peaks selected from Table 5.

TABLE 8
XRPD Peak List of ORO Form D

| | Position (°2- | Intensity | |
|---|---|---|---|
| Peak | theta) | (Counts) | Rel. Int. (%) |
| 1 | 5.2 | 6938.0 | 100.0 |
| 2 | 7.9 | 1974.0 | 28.5 |
| 3 | 12.1 | 2688.0 | 38.7 |
| 4 | 14.0 | 1441.0 | 20.8 |
| 5 | 14.9 | 1664.0 | 24.0 |
| 6 | 15.1 | 1604.0 | 23.1 |
| 7 | 16.1 | 2657.0 | 38.3 |
| 8 | 17.4 | 2710.0 | 39.1 |
| 9 | 18.2 | 2080.0 | 30.0 |
| 10 | 19.1 | 1676.0 | 24.2 |
| 11 | 20.2 | 1680.0 | 24.2 |
| 12 | 21.0 | 1639.0 | 23.6 |
| 13 | 21.8 | 1665.0 | 24.0 |
| 14 | 22.6 | 1668.0 | 24.0 |
| 15 | 23.7 | 2774.0 | 40.0 |
| 16 | 24.3 | 1295.0 | 18.7 |
| 17 | 27.9 | 4230.0 | 61.0 |
| 18 | 28.9 | 1649.0 | 23.8 |
| 19 | 29.1 | 1013.0 | 14.6 |

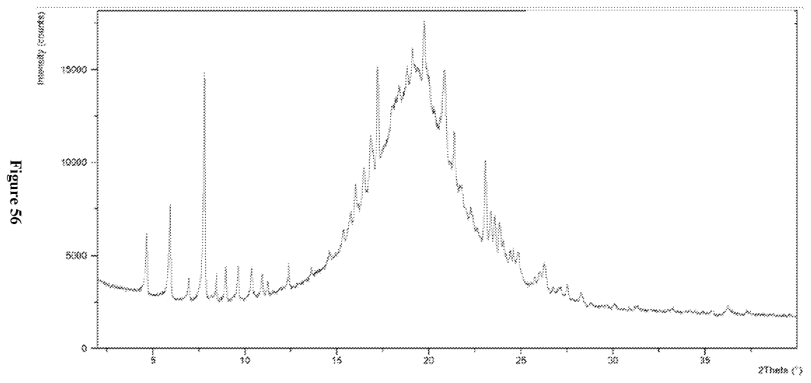

In some embodiments, the crystalline form exhibits an X-ray powder diffraction pattern substantially the same as depicted in FIG. 55.

### GEN Form B

In one aspect, a gentisic salt of a compound of Formula (I):

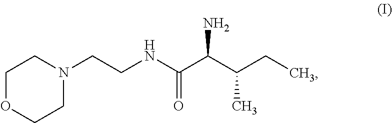 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 18.6±0.2, 18.9±0.2, and 19.5±0.2.

In some embodiments, such a crystalline form exhibits an X-ray powder diffraction pattern comprising peaks at the following diffraction angles (2θ): 17.0±0.2, 18.6±0.2, 18.9±0.2, 19.5±0.2, and 20.6±0.2.

In some embodiments, such a crystalline form exhibits an X-ray powder diffraction pattern comprising peaks at the following diffraction angles (2θ): 7.7±0.2, 16.3±0.2, 16.7±0.2, 17.0±0.2, 18.6±0.2, 18.9±0.2, 19.5±0.2, 20.6±0.2, 21.2±0.2, and 22.8±0.2.

In some embodiments, the crystalline form exhibits an X-ray powder diffraction pattern comprising some peaks selected from Table 9.

TABLE 9
XRPD Peak List of GEN Form B

| | Position (°2- | Intensity | |
|---|---|---|---|
| Peak | theta) | (Counts) | Rel. Int. (%) |
| 1 | 4.6 | 2370.0 | 32.2 |
| 2 | 5.9 | 3017.0 | 41.0 |
| 3 | 6.9 | 1531.0 | 20.8 |
| 4 | 7.7 | 5467.0 | 74.3 |
| 5 | 8.4 | 1557.0 | 21.1 |
| 6 | 8.9 | 1670.0 | 22.7 |
| 7 | 9.5 | 1752.0 | 23.8 |
| 8 | 10.3 | 1781.0 | 24.2 |
| 9 | 10.8 | 1631.0 | 22.2 |
| 10 | 12.3 | 1875.0 | 25.5 |
| 11 | 15.2 | 2672.0 | 36.3 |
| 12 | 15.6 | 3119.0 | 42.4 |
| 13 | 15.9 | 3638.0 | 49.4 |
| 14 | 16.3 | 3949.0 | 53.6 |
| 15 | 16.7 | 4796.0 | 65.1 |
| 16 | 17.0 | 6091.0 | 82.7 |
| 17 | 18.6 | 6429.0 | 87.3 |
| 18 | 18.9 | 6891.0 | 93.6 |
| 19 | 19.5 | 7362.0 | 100.0 |
| 20 | 20.6 | 6234.0 | 84.7 |
| 21 | 21.2 | 4710.0 | 64.0 |
| 22 | 22.8 | 3906.0 | 53.1 |
| 23 | 23.1 | 2954.0 | 40.1 |
| 24 | 23.4 | 2797.0 | 38.0 |
| 25 | 23.6 | 2794.0 | 38.0 |

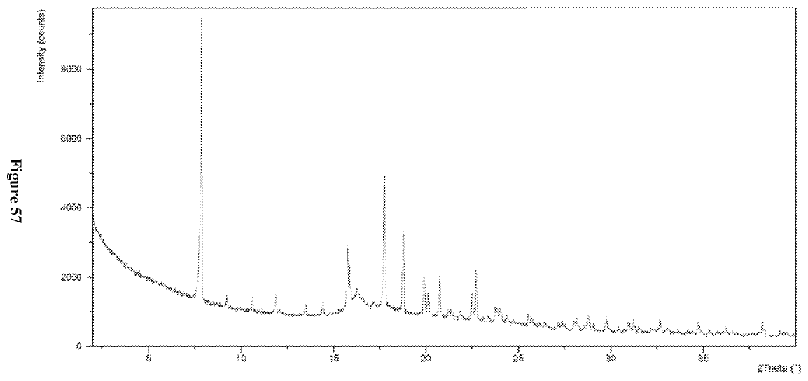

In some embodiments, the crystalline form exhibits an X-ray powder diffraction pattern substantially the same as depicted in FIG. 56.

### TAR Form C

In one aspect, a tartaric salt of a compound of Formula (I):

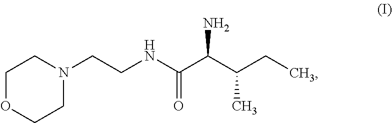 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 7.8±0.2, 17.6±0.2, and 18.6±0.2.

In some embodiments, such a crystalline form exhibits an X-ray powder diffraction pattern comprising peaks at the following diffraction angles (2θ): 7.8±0.2, 15.6±0.2, 15.7±0.2, 17.6±0.2, and 18.6±0.2.

In some embodiments, such a crystalline form exhibits an X-ray powder diffraction pattern comprising peaks at the following diffraction angles (2θ): 7.8±0.2, 15.6±0.2, 15.7±0.2, 16.1±0.2, 17.6±0.2, 18.6±0.2, 19.7±0.2, 19.9±0.2, 20.5±0.2, and 22.5±0.2.

In some embodiments, the crystalline form exhibits an X-ray powder diffraction pattern comprising some peaks selected from Table 10.

TABLE 10
XRPD Peak List of TAR Form C

| | Position (°2- | Intensity | |
|---|---|---|---|
| Peak | theta) | (Counts) | Rel. Int. (%) |
| 1 | 7.8 | 9470.0 | 100.0 |
| 2 | 9.2 | 1494.0 | 15.8 |
| 3 | 10.5 | 1453.0 | 15.3 |
| 4 | 11.8 | 1478.0 | 15.6 |
| 5 | 13.4 | 1237.0 | 13.1 |
| 6 | 14.3 | 1296.0 | 13.7 |
| 7 | 15.6 | 2930.0 | 30.9 |
| 8 | 15.7 | 2377.0 | 25.1 |
| 9 | 16.1 | 1688.0 | 17.8 |
| 10 | 17.6 | 4920.0 | 52.0 |
| 11 | 18.6 | 3382.0 | 35.7 |
| 12 | 19.7 | 2173.0 | 22.9 |
| 13 | 19.9 | 1576.0 | 16.6 |
| 14 | 20.5 | 2059.0 | 21.7 |
| 15 | 22.3 | 1568.0 | 16.6 |
| 16 | 22.5 | 2231.0 | 23.6 |
| 17 | 23.5 | 1166.0 | 12.3 |
| 18 | 23.8 | 1106.0 | 11.7 |
| 19 | 25.3 | 966.0 | 10.2 |
| 20 | 27.9 | 841.0 | 8.9 |
| 21 | 28.5 | 882.0 | 9.3 |
| 22 | 29.4 | 867.0 | 9.2 |
| 23 | 30.9 | 808.0 | 8.5 |
| 24 | 32.3 | 775.0 | 8.2 |
| 25 | 34.4 | 688.0 | 7.3 |
| 26 | 37.8 | 748.0 | 7.9 |

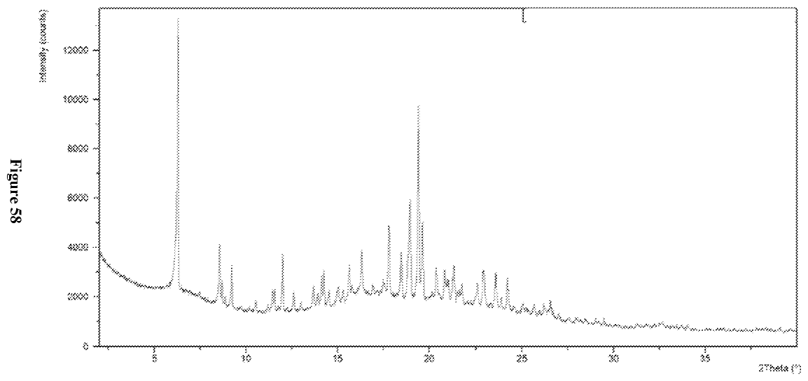

In some embodiments, the crystalline form exhibits an X-ray powder diffraction pattern substantially the same as depicted in FIG. 57.

### 2-MXB Form A

In one aspect, a 2-methoxybenzoic salt of a compound of Formula (I):

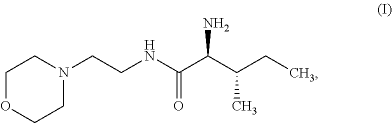 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 6.2±0.2, 18.8±0.2, 19.2±0.2.

In some embodiments, such a crystalline form exhibits an X-ray powder diffraction pattern comprising peaks at the following diffraction angles (2θ): 6.2±0.2, 17.6±0.2, 18.8±0.2, 19.2±0.2, and 19.4±0.2.

In some embodiments, such a crystalline form exhibits an X-ray powder diffraction pattern comprising peaks at the following diffraction angles (2θ): 6.2±0.2, 8.5±0.2, 11.9±0.2, 15.5±0.2, 16.2±0.2, 17.6±0.2, 18.3±0.2, 18.8±0.2, 19.2±0.2, and 19.4±0.2.

In some embodiments, the crystalline form exhibits an X-ray powder diffraction pattern comprising some peaks selected from Table 11.

TABLE 11
XRPD Peak List of 2-MXB Form A

| | Position (°2- | Intensity | |
|---|---|---|---|
| Peak | theta) | (Counts) | Rel. Int. (%) |
| 1 | 6.2 | 13291.0 | 100.0 |
| 2 | 8.5 | 4174.0 | 31.4 |
| 3 | 8.6 | 2675.0 | 20.1 |
| 4 | 9.1 | 3282.0 | 24.7 |
| 5 | 11.3 | 2283.0 | 17.2 |
| 6 | 11.4 | 2321.0 | 17.5 |
| 7 | 11.9 | 3767.0 | 28.3 |
| 8 | 13.6 | 2448.0 | 18.4 |
| 10 | 14.1 | 3119.0 | 23.5 |
| 11 | 15.5 | 3314.0 | 24.9 |
| 12 | 16.2 | 3884.0 | 29.2 |
| 13 | 17.3 | 2726.0 | 20.5 |
| 14 | 17.6 | 4919.0 | 37.0 |
| 15 | 18.3 | 3820.0 | 28.7 |
| 16 | 18.8 | 5953.0 | 44.8 |
| 17 | 19.2 | 9790.0 | 73.7 |
| 18 | 19.4 | 5062.0 | 38.1 |
| 19 | 20.2 | 3216.0 | 24.2 |
| 20 | 20.6 | 3119.0 | 23.5 |
| 21 | 20.8 | 2713.0 | 20.4 |
| 22 | 21.1 | 3271.0 | 24.6 |
| 23 | 21.5 | 2558.0 | 19.2 |
| 24 | 22.3 | 2589.0 | 19.5 |
| 25 | 22.7 | 3129.0 | 23.5 |
| 26 | 23.3 | 2985.0 | 22.5 |
| 27 | 24.0 | 2806.0 | 21.1 |

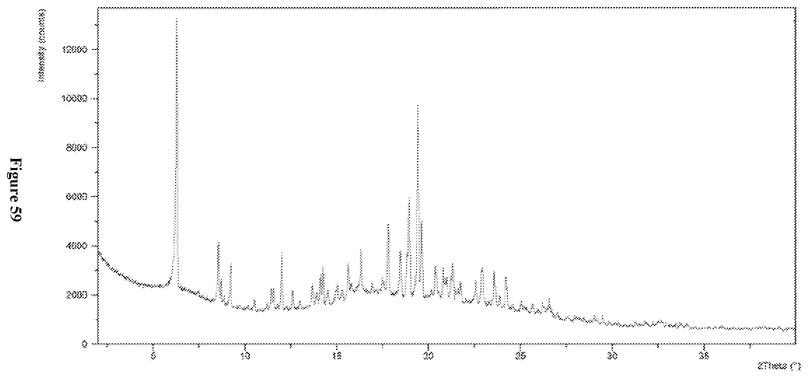

In some embodiments, the crystalline form exhibits an X-ray powder diffraction pattern substantially the same as depicted in FIG. 58.

### 3-MXB Form A

In one aspect, a 3-methoxybenzoic salt of a compound of Formula (I):

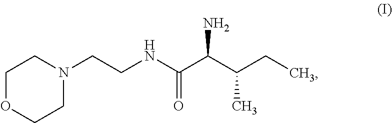 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 8.1±0.2, 17.1±0.2, and 22.0±0.2.

In some embodiments, such a crystalline form exhibits an X-ray powder diffraction pattern comprising peaks at the following diffraction angles (2θ): 8.1±0.2, 17.1±0.2, 18.9±0.2, 22±0.2, and 22.2±0.2.

In some embodiments, such a crystalline form exhibits an X-ray powder diffraction pattern comprising peaks at the following diffraction angles (2θ): 8.1±0.2, 14.3±0.2, 15.0±0.2, 15.7±0.2, 17.1±0.2, 18.9±0.2, 21.7±0.2, 22±0.2, 22.2±0.2, and 22.7±0.2.

In some embodiments, the crystalline form exhibits an X-ray powder diffraction pattern comprising some peaks selected from Table 12.

TABLE 12
XRPD Peak List of 3-MXB Form A

| | Position (°2- | Intensity | |
|---|---|---|---|
| Peak | theta) | (Counts) | Rel. Int. (%) |
| 1 | 4.7 | 3061.0 | 20.6 |
| 2 | 8.1 | 14867.0 | 100.0 |
| 3 | 8.5 | 2906.0 | 19.5 |
| 4 | 9.3 | 2385.0 | 16.0 |
| 5 | 10.8 | 2387.0 | 16.1 |
| 6 | 12.3 | 2745.0 | 18.5 |
| 7 | 14.3 | 4124.0 | 27.7 |
| 8 | 15.0 | 5755.0 | 38.7 |
| 9 | 15.7 | 4263.0 | 28.7 |
| 10 | 17.1 | 7345.0 | 49.4 |
| 11 | 18.3 | 4088.0 | 27.5 |
| 12 | 18.7 | 3345.0 | 22.5 |
| 13 | 18.9 | 6147.0 | 41.3 |
| 14 | 20.1 | 3319.0 | 22.3 |
| 15 | 20.4 | 3481.0 | 23.4 |
| 16 | 21.7 | 4929.0 | 33.2 |
| 17 | 22.0 | 8616.0 | 58.0 |
| 18 | 22.2 | 6550.0 | 44.1 |
| 19 | 22.7 | 4731.0 | 31.8 |
| 20 | 23.5 | 2429.0 | 16.3 |
| 21 | 23.5 | 2429.0 | 16.3 |
| 22 | 23.6 | 2286.0 | 15.4 |
| 23 | 24.9 | 2193.0 | 14.8 |
| 24 | 25.9 | 2611.0 | 17.6 |

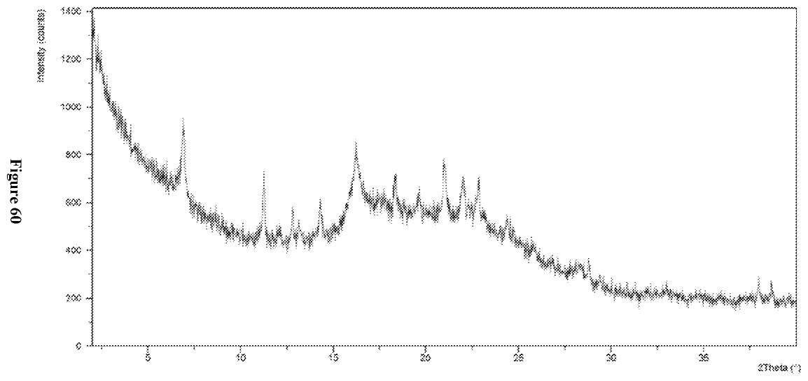

In some embodiments, the crystalline form exhibits an X-ray powder diffraction pattern substantially the same as depicted in FIG. 59.

### FUM Form A

In one aspect, a fumaric salt of a compound of Formula (I):

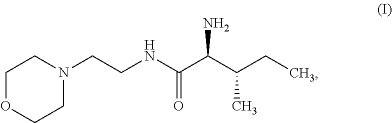 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 6.9±0.2, 16.1±0.2, and 20.8±0.2.

In some embodiments, such a crystalline form exhibits an X-ray powder diffraction pattern comprising peaks at the following diffraction angles (2θ): 6.9±0.2, 11.2±0.2, 16.1±0.2, 18.2±0.2, and 20.8±0.2.

In some embodiments, such a crystalline form exhibits an X-ray powder diffraction pattern comprising peaks at the following diffraction angles (2θ): 6.9±0.2, 11.2±0.2, 12.7±0.2, 14.2±0.2, 16.1±0.2, 18.2±0.2, 19.5±0.2, 20.8±0.2, 21.8±0.2, and 22.6±0.2.

In some embodiments, the crystalline form exhibits an X-ray powder diffraction pattern comprising some peaks selected from Table 13.

TABLE 13
XRPD Peak List of FUM Form A

| | Position (°2- | Intensity | |
|---|---|---|---|
| Peak | theta) | (Counts) | Rel. Int. (%) |
| 1 | 6.9 | 956.0 | 100.0 |
| 2 | 11.2 | 733.0 | 76.7 |
| 3 | 12.7 | 583.0 | 61.0 |
| 4 | 14.2 | 618.0 | 64.6 |
| 5 | 16.1 | 863.0 | 90.3 |
| 6 | 18.2 | 728.0 | 76.2 |
| 7 | 19.5 | 666.0 | 69.7 |
| 8 | 20.8 | 786.0 | 82.2 |
| 9 | 21.8 | 709.0 | 74.2 |
| 10 | 22.6 | 708.0 | 74.1 |

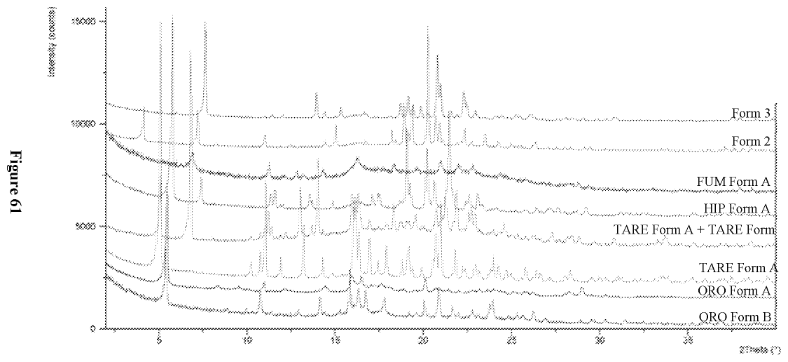

In some embodiments, the crystalline form exhibits an X-ray powder diffraction pattern substantially the same as depicted in FIG. 60.

### HIP Form A

In one aspect, a hippuric salt of a compound of Formula (I):

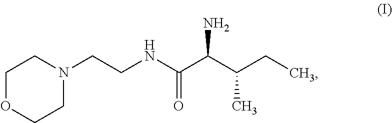 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 5.7±0.2, 18.9±0.2, and 21.2±0.2.

In some embodiments, such a crystalline form exhibits an X-ray powder diffraction pattern comprising peaks at the following diffraction angles (2θ): 5.7±0.2, 7.3±0.2, 18.9±0.2, 20.0±0.2, and 21.2±0.2.

In some embodiments, such a crystalline form exhibits an X-ray powder diffraction pattern comprising peaks at the following diffraction angles (2θ): 5.7±0.2, 7.3±0.2, 11.5±0.2, 17.2±0.2, 18.9±0.2, 19.1±0.2, 20.0±0.2, 20.4±0.2, 21.2±0.2, and 22.8±0.2.

In some embodiments, the crystalline form exhibits an X-ray powder diffraction pattern comprising some peaks selected from Table 14.

TABLE 14
XRPD Peak List of HIP Form A

| | Position (°2- | Intensity | |
|---|---|---|---|
| Peak | theta) | (Counts) | Rel. Int. (%) |
| 1 | 5.7 | 14958.0 | 100.0 |
| 2 | 7.3 | 3179.0 | 21.3 |
| 3 | 11.3 | 2000.0 | 13.4 |
| 4 | 11.5 | 2276.0 | 15.2 |
| 5 | 13.4 | 1916.0 | 12.8 |
| 6 | 13.6 | 1485.0 | 9.9 |
| 7 | 14.7 | 1443.0 | 9.6 |
| 8 | 16.9 | 1903.0 | 12.7 |
| 9 | 17.2 | 2016.0 | 13.5 |
| 10 | 17.3 | 1954.0 | 13.1 |
| 11 | 18.9 | 6850.0 | 45.8 |
| 12 | 19.1 | 2692.0 | 18.0 |
| 13 | 20.0 | 5268.0 | 35.2 |
| 14 | 20.4 | 2928.0 | 19.6 |
| 15 | 21.2 | 8016.0 | 53.6 |
| 16 | 22.2 | 1438.0 | 9.6 |
| 17 | 22.4 | 1823.0 | 12.2 |
| 18 | 22.8 | 2019.0 | 13.5 |
| 19 | 28.9 | 1014.0 | 6.8 |

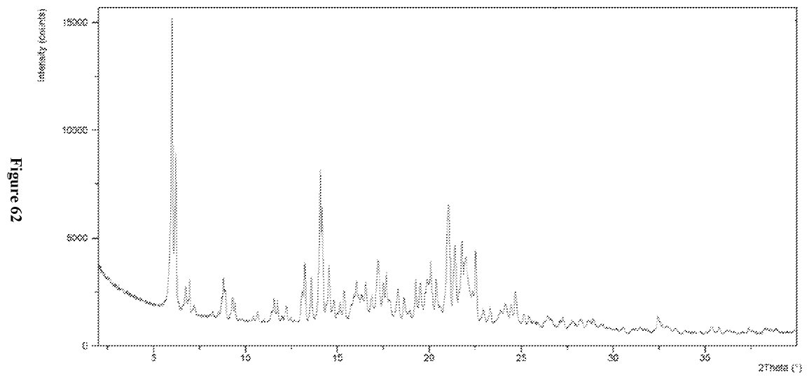

In some embodiments, the crystalline form exhibits an X-ray powder diffraction pattern substantially the same as depicted for HIP Form A in FIG. 61.

### HIP Form B

In one aspect, a hippuric salt of a compound of Formula (I):

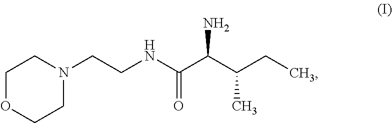 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 5.9±0.2, 6.2±0.2, and 13.9±0.2.

In some embodiments, such a crystalline form exhibits an X-ray powder diffraction pattern comprising peaks at the following diffraction angles (2θ): 5.9±0.2, 6.2±0.2, 13.9±0.2, 20.8±0.2, and 21.6±0.2.

In some embodiments, such a crystalline form exhibits an X-ray powder diffraction pattern comprising peaks at the following diffraction angles (2θ): 5.9±0.2, 6.2±0.2, 13.9±0.2, 17.0±0.2, 19.9±0.2, 20.8±0.2, 21.2±0.2, 21.6±0.2, 21.8±0.2, and 22.3±0.2.

In some embodiments, the crystalline form exhibits an X-ray powder diffraction pattern comprising some peaks selected from Table 15.

TABLE 15
XRPD Peak List of HIP Form B

| | Position (°2- | Intensity | |
|---|---|---|---|
| Peak | theta) | (Counts) | Rel. Int. (%) |
| 1 | 5.9 | 15228.0 | 100.0 |
| 2 | 6.2 | 8954.0 | 58.8 |
| 3 | 6.7 | 2798.0 | 18.4 |
| 4 | 6.9 | 3142.0 | 20.6 |
| 5 | 8.7 | 3161.0 | 20.8 |
| 6 | 9.2 | 2245.0 | 14.7 |
| 7 | 9.4 | 2039.0 | 13.4 |
| 8 | 11.4 | 2224.0 | 14.6 |
| 9 | 11.6 | 2114.0 | 13.9 |
| 10 | 12.1 | 1903.0 | 12.5 |
| 11 | 13.1 | 3903.0 | 25.6 |
| 12 | 13.4 | 3219.0 | 21.1 |
| 13 | 13.9 | 8178.0 | 53.7 |
| 14 | 14.4 | 3740.0 | 24.6 |
| 15 | 14.7 | 2164.0 | 14.2 |
| 16 | 15.0 | 2025.0 | 13.3 |
| 17 | 15.2 | 2616.0 | 17.2 |
| 18 | 15.9 | 3054.0 | 20.1 |
| 19 | 16.1 | 2410.0 | 15.8 |
| 20 | 16.4 | 2982.0 | 19.6 |
| 21 | 16.7 | 2404.0 | 15.8 |
| 22 | 17.0 | 4023.0 | 26.4 |
| 23 | 17.3 | 2966.0 | 19.5 |
| 24 | 17.5 | 3441.0 | 22.6 |
| 25 | 18.1 | 2664.0 | 17.5 |
| 26 | 18.5 | 2261.0 | 14.8 |
| 27 | 19.1 | 3083.0 | 20.2 |
| 28 | 19.3 | 2985.0 | 19.6 |
| 29 | 19.7 | 3092.0 | 20.3 |
| 30 | 19.9 | 3981.0 | 26.1 |
| 31 | 20.2 | 3166.0 | 20.8 |
| 32 | 20.8 | 6625.0 | 43.5 |
| 33 | 21.2 | 4704.0 | 30.9 |
| 34 | 21.6 | 4867.0 | 32.0 |
| 35 | 21.8 | 4152.0 | 27.3 |
| 36 | 22.3 | 4481.0 | 29.4 |
| 37 | 22.7 | 1742.0 | 11.4 |
| 38 | 23.1 | 1826.0 | 12.0 |
| 39 | 23.6 | 1706.0 | 11.2 |
| 40 | 23.9 | 2000.0 | 13.1 |
| 41 | 24.2 | 1946.0 | 12.8 |
| 42 | 24.4 | 2562.0 | 16.8 |
| 43 | 24.9 | 1542.0 | 10.1 |

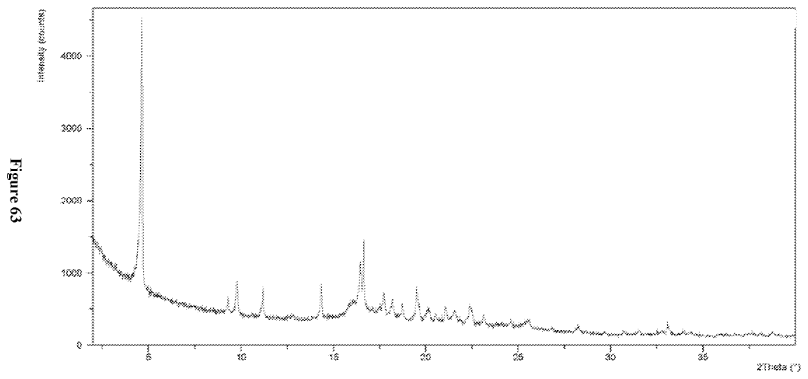

In some embodiments, the crystalline form exhibits an X-ray powder diffraction pattern substantially the same as depicted in FIG. 62.

### MUC Form A

In one aspect, a mucic salt of a compound of Formula (I):

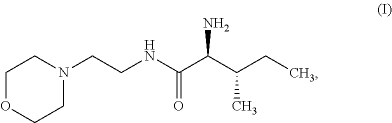 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 4.6±0.2, 16.3±0.2, and 16.5±0.2.

In some embodiments, such a crystalline form exhibits an X-ray powder diffraction pattern comprising peaks at the following diffraction angles (2θ): 4.6±0.2, 9.7±0.2, 14.2±0.2, 16.3±0.2, and 16.5±0.2.

In some embodiments, such a crystalline form exhibits an X-ray powder diffraction pattern comprising peaks at the following diffraction angles (2θ): 4.6±0.2, 9.2±0.2, 9.7±0.2, 11.1±0.2, 14.2±0.2, 16.3±0.2, 16.5±0.2, 17.3±0.2, 17.5±0.2, 18.0±0.2, and 19.3±0.2.

In some embodiments, the crystalline form exhibits an X-ray powder diffraction pattern comprising some peaks selected from Table 16.

TABLE 16
XRPD Peak List of MUC Form A

| | Position (°2- | Intensity | |
|---|---|---|---|
| Peak | theta) | (Counts) | Rel. Int. (%) |
| 1 | 4.6 | 4525.0 | 100.0 |
| 2 | 9.2 | 673.0 | 14.9 |
| 3 | 9.7 | 895.0 | 19.8 |
| 4 | 11.1 | 825.0 | 18.2 |
| 5 | 14.2 | 851.0 | 18.8 |
| 6 | 16.3 | 1146.0 | 25.3 |
| 7 | 16.5 | 1479.0 | 32.7 |
| 8 | 17.3 | 577.0 | 12.8 |
| 9 | 17.5 | 723.0 | 16.0 |
| 10 | 18.0 | 654.0 | 14.5 |
| 11 | 18.5 | 575.0 | 12.7 |
| 12 | 19.3 | 807.0 | 17.8 |
| 13 | 19.9 | 529.0 | 11.7 |
| 14 | 20.9 | 546.0 | 12.1 |
| 15 | 22.2 | 566.0 | 12.5 |
| 16 | 32.7 | 321.0 | 7.1 |

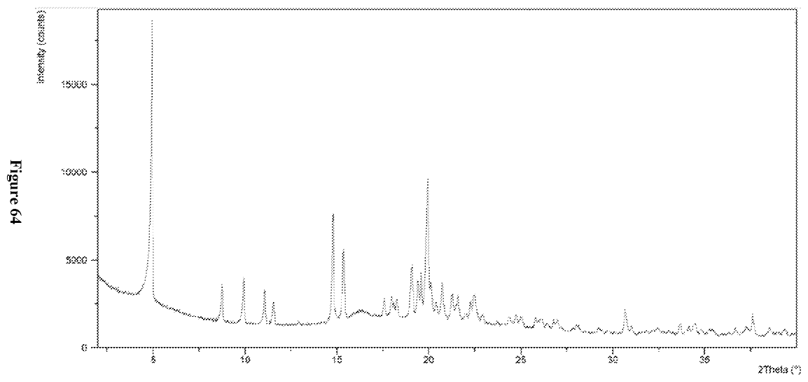

In some embodiments, the crystalline form exhibits an X-ray powder diffraction pattern substantially the same as depicted in FIG. 63.

### MUC Form B

In one aspect, a mucic salt of a compound of Formula (I):

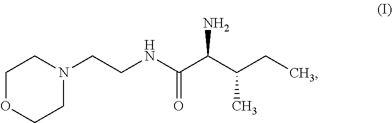 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 4.9±0.2, 14.6±0.2, and 19.7±0.2.

In some embodiments, such a crystalline form exhibits an X-ray powder diffraction pattern comprising peaks at the following diffraction angles (2θ): 4.9±0.2, 14.6±0.2, 15.2±0.2, 18.9±0.2, and 19.7±0.2.

In some embodiments, such a crystalline form exhibits an X-ray powder diffraction pattern comprising peaks at the following diffraction angles (2θ): 4.9±0.2, 9.8±0.2, 14.6±0.2, 15.2±0.2, 18.9±0.2, 19.2±0.2, 19.4±0.2, 19.7±0.2, 19.9±0.2, and 20.5±0.2.

In some embodiments, the crystalline form exhibits an X-ray powder diffraction pattern comprising some peaks selected from Table 17.

TABLE 17
XRPD Peak List of MUC Form B

| | Position (°2- | Intensity | |
|---|---|---|---|
| Peak | theta) | (Counts) | Rel. Int. (%) |
| 1 | 4.9 | 18700.0 | 100.0 |
| 2 | 8.7 | 3630.0 | 19.4 |
| 3 | 9.8 | 4002.0 | 21.4 |
| 4 | 11.0 | 3306.0 | 17.7 |
| 5 | 11.4 | 2718.0 | 14.5 |
| 6 | 14.6 | 7687.0 | 41.1 |
| 7 | 15.2 | 5628.0 | 30.1 |
| 8 | 17.4 | 2805.0 | 15.0 |
| 9 | 17.8 | 3023.0 | 16.2 |
| 10 | 18.1 | 2776.0 | 14.8 |
| 11 | 18.9 | 4730.0 | 25.3 |
| 12 | 19.2 | 3864.0 | 20.7 |
| 13 | 19.4 | 4379.0 | 23.4 |
| 14 | 19.7 | 9683.0 | 51.8 |
| 15 | 19.9 | 3797.0 | 20.3 |
| 16 | 20.2 | 2629.0 | 14.1 |
| 17 | 20.5 | 3829.0 | 20.5 |
| 18 | 21.1 | 3060.0 | 16.4 |
| 19 | 21.4 | 3011.0 | 16.1 |
| 20 | 22.0 | 2666.0 | 14.3 |
| 21 | 22.2 | 3107.0 | 16.6 |
| 22 | 24.5 | 1987.0 | 10.6 |
| 23 | 24.8 | 1828.0 | 9.8 |
| 24 | 25.5 | 1731.0 | 9.3 |
| 25 | 25.8 | 1665.0 | 8.9 |
| 26 | 26.5 | 1615.0 | 8.6 |
| 27 | 26.7 | 1632.0 | 8.7 |
| 28 | 30.4 | 2208.0 | 11.8 |
| 29 | 33.3 | 1418.0 | 7.6 |
| 30 | 34.1 | 1412.0 | 7.6 |
| 31 | 37.2 | 1945.0 | 10.4 |

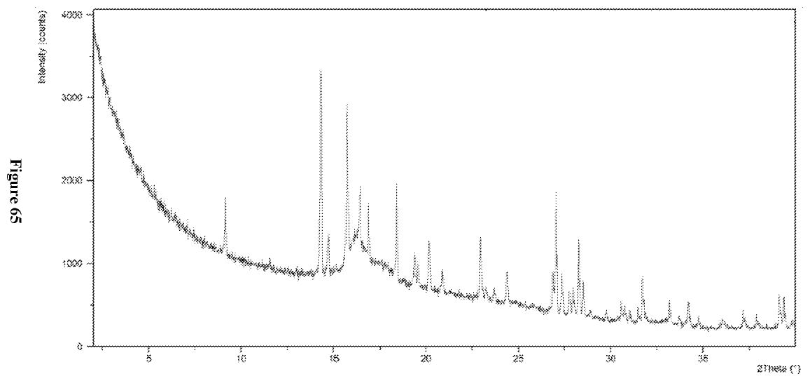

In some embodiments, the crystalline form exhibits an X-ray powder diffraction pattern substantially the same as depicted in FIG. 64.

### MUC Form C

In one aspect, a mucic salt of a compound of Formula (I):

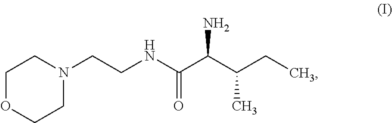 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 14.2±0.2, 15.6±0.2, and 18.2±0.2.

In some embodiments, such a crystalline form exhibits an X-ray powder diffraction pattern comprising peaks at the following diffraction angles (2θ): 14.2±0.2, 15.6±0.2, 16.2±0.2, 18.2±0.2, 26.7±0.2.

In some embodiments, such a crystalline form exhibits an X-ray powder diffraction pattern comprising peaks at the following diffraction angles (2θ): 9.1±0.2, 14.2±0.2, 14.6±0.2, 15.6±0.2, 16.2±0.2, 16.7±0.2, 18.2±0.2, 22.7±0.2, 26.7±0.2, and 28.0±0.2.

In some embodiments, the crystalline form exhibits an X-ray powder diffraction pattern comprising some peaks selected from Table 18.

TABLE 18
XRPD Peak List of MUC Form C

| | Position (°2- | Intensity | |
|---|---|---|---|
| Peak | theta) | (Counts) | Rel. Int. (%) |
| 1 | 9.1 | 1808.0 | 54.1 |
| 2 | 14.2 | 3341.0 | 100.0 |
| 3 | 14.6 | 1363.0 | 40.8 |
| 4 | 15.6 | 2923.0 | 87.5 |
| 5 | 16.2 | 1934.0 | 57.9 |
| 6 | 16.7 | 1731.0 | 51.8 |
| 7 | 18.2 | 1976.0 | 59.1 |
| 8 | 19.2 | 1131.0 | 33.9 |
| 9 | 19.4 | 1029.0 | 30.8 |
| 10 | 19.9 | 1277.0 | 38.2 |
| 11 | 20.7 | 930.0 | 27.8 |
| 12 | 22.7 | 1311.0 | 39.2 |
| 13 | 24.1 | 901.0 | 27.0 |
| 14 | 26.6 | 907.0 | 27.1 |
| 15 | 26.7 | 1886.0 | 56.5 |
| 16 | 27.1 | 873.0 | 26.1 |
| 17 | 27.5 | 670.0 | 20.1 |
| 18 | 27.7 | 707.0 | 21.2 |
| 19 | 28.0 | 1297.0 | 38.8 |
| 20 | 28.2 | 803.0 | 24.0 |
| 21 | 30.3 | 552.0 | 16.5 |
| 22 | 31.4 | 849.0 | 25.4 |
| 23 | 32.8 | 554.0 | 16.6 |
| 24 | 33.9 | 545.0 | 16.3 |
| 25 | 38.7 | 631.0 | 18.9 |
| 26 | 39.0 | 603.0 | 18.0 |

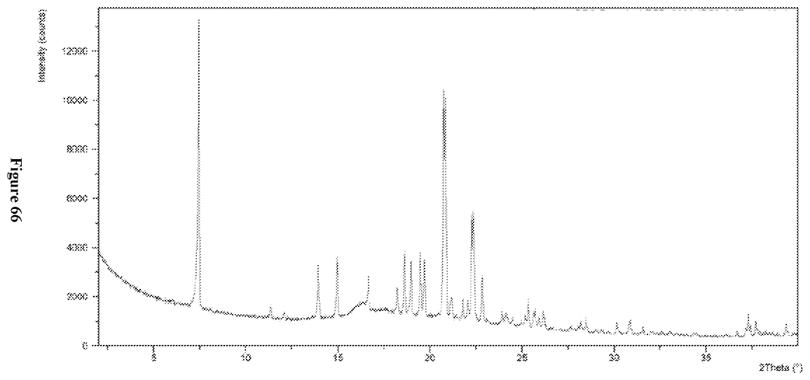

In some embodiments, the crystalline form exhibits an X-ray powder diffraction pattern substantially the same as depicted in FIG. 65.

### PRO Form A

In one aspect, a propionic salt of a compound of Formula (I):

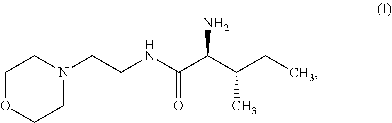 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 7.4±0.2, 20.5±0.2, and 20.6±0.2.

In some embodiments, such a crystalline form exhibits an X-ray powder diffraction pattern comprising peaks at the following diffraction angles (2θ): 7.4±0.2, 20.5±0.2, 20.6±0.2, 22.0±0.2, and 22.1±0.2

In some embodiments, such a crystalline form exhibits an X-ray powder diffraction pattern comprising peaks at the following diffraction angles (2θ): 7.4±0.2, 14.8±0.2, 18.4±0.2, 18.8±0.2, 19.3±0.2, 19.5±0.2, 20.5±0.2, 20.6±0.2, 22.0±0.2, and 22.1±0.2.

In some embodiments, the crystalline form exhibits an X-ray powder diffraction pattern comprising some peaks selected from Table 19.

TABLE 19
XRPD Peak List of PRO Form A

| | Position (°2- | Intensity | |
|---|---|---|---|
| Peak | theta) | (Counts) | Rel. Int. (%) |
| 1 | 7.4 | 13345.0 | 100.0 |
| 2 | 11.3 | 1661.0 | 12.4 |
| 3 | 12.0 | 1420.0 | 10.6 |
| 4 | 13.8 | 3349.0 | 25.1 |
| 5 | 14.8 | 3618.0 | 27.1 |
| 6 | 16.5 | 2891.0 | 21.7 |
| 7 | 18.0 | 2390.0 | 17.9 |
| 8 | 18.4 | 3838.0 | 28.8 |
| 9 | 18.8 | 3461.0 | 25.9 |
| 10 | 19.3 | 3801.0 | 28.5 |
| 11 | 19.5 | 3522.0 | 26.4 |
| 12 | 20.5 | 10436.0 | 78.2 |
| 13 | 20.6 | 10147.0 | 76.0 |
| 14 | 21.0 | 1968.0 | 14.7 |
| 15 | 21.6 | 1916.0 | 14.4 |
| 16 | 21.6 | 1916.0 | 14.4 |
| 17 | 21.8 | 1866.0 | 14.0 |
| 18 | 22.0 | 5334.0 | 40.0 |
| 19 | 22.1 | 5483.0 | 41.1 |
| 20 | 22.6 | 2857.0 | 21.4 |
| 21 | 23.7 | 1426.0 | 10.7 |
| 22 | 23.9 | 1398.0 | 10.5 |
| 23 | 24.2 | 1157.0 | 8.7 |
| 24 | 24.9 | 1279.0 | 9.6 |
| 25 | 25.1 | 1915.0 | 14.3 |
| 26 | 25.5 | 1491.0 | 11.2 |
| 27 | 25.7 | 1174.0 | 8.8 |
| 28 | 25.9 | 1451.0 | 10.9 |
| 29 | 27.9 | 1039.0 | 7.8 |
| 30 | 28.2 | 1145.0 | 8.6 |
| 31 | 29.9 | 946.0 | 7.1 |
| 32 | 30.6 | 1072.0 | 8.0 |
| 33 | 36.9 | 1353.0 | 10.1 |

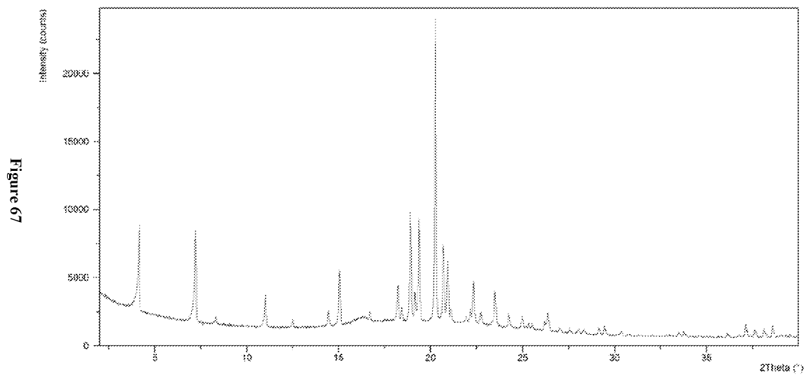

In some embodiments, the crystalline form exhibits an X-ray powder diffraction pattern substantially the same as depicted in FIG. 66.

### Form 2

In one aspect, a polymorph of the compound of Formula (I):

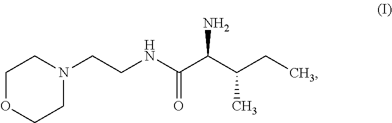 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 19.7±0.2, 19.2±0.2, and 20.1±0.2

In some embodiments, such a crystalline form exhibits an X-ray powder diffraction pattern comprising peaks at the following diffraction angles (2θ): 4.1±0.2, 7.1±0.2, 18.7±0.2, 19.2±0.2, and 20.1±0.2.

In some embodiments, such a crystalline form exhibits an X-ray powder diffraction pattern comprising peaks at the following diffraction angles (2θ): 4.1±0.2, 7.1±0.2, 14.9±0.2, 18.7±0.2, 19.2±0.2, 20.1±0.2, 20.5±0.2, 20.7±0.2, and 22.1±0.2.

In some embodiments, the crystalline form exhibits an X-ray powder diffraction pattern comprising some peaks selected from Table 20.

TABLE 20
XRPD Peak List of Form 2

| | Position (°2- | Intensity | |
|---|---|---|---|
| Peak | theta) | (Counts) | Rel. Int. (%) |
| 1 | 4.1 | 8887.0 | 37.0 |
| 2 | 7.1 | 8475.0 | 35.2 |
| 3 | 8.2 | 2181.0 | 9.1 |
| 4 | 10.9 | 3829.0 | 15.9 |
| 5 | 12.4 | 1932.0 | 8.0 |
| 6 | 14.3 | 2564.0 | 10.7 |
| 7 | 14.9 | 5571.0 | 23.2 |
| 8 | 16.5 | 2553.0 | 10.6 |
| 9 | 18.0 | 4481.0 | 18.6 |
| 10 | 18.2 | 2820.0 | 11.7 |
| 11 | 18.7 | 9812.0 | 40.8 |
| 12 | 19.0 | 4063.0 | 16.9 |
| 13 | 19.2 | 9286.0 | 38.6 |
| 14 | 20.1 | 24047.0 | 100.0 |
| 15 | 20.5 | 7466.0 | 31.0 |
| 16 | 20.7 | 6240.0 | 25.9 |
| 17 | 20.9 | 2657.0 | 11.0 |
| 18 | 21.7 | 2145.0 | 8.9 |
| 19 | 21.9 | 2717.0 | 11.3 |
| 20 | 22.1 | 4763.0 | 19.8 |
| 21 | 22.5 | 2463.0 | 10.2 |
| 22 | 23.3 | 4061.0 | 16.9 |
| 23 | 24.0 | 2370.0 | 9.9 |
| 24 | 24.7 | 2148.0 | 8.9 |
| 25 | 25.9 | 1813.0 | 7.5 |
| 26 | 26.1 | 2444.0 | 10.2 |
| 27 | 28.9 | 1380.0 | 5.7 |
| 28 | 29.2 | 1488.0 | 6.2 |
| 29 | 36.7 | 1613.0 | 6.7 |
| 30 | 37.2 | 1288.0 | 5.4 |
| 31 | 37.7 | 1233.0 | 5.1 |
| 32 | 38.2 | 1512.0 | 6.3 |

In some embodiments, the crystalline form exhibits an X-ray powder diffraction pattern substantially the same as depicted in Figure.

### Form 3

In one aspect, a polymorph of the compound of Formula (I):

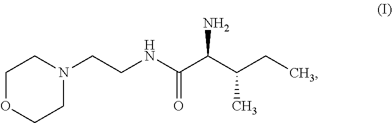 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 7.6±0.2, 20.6±0.2, and 20.8±0.2.

In some embodiments, such a crystalline form exhibits an X-ray powder diffraction pattern comprising peaks at the following diffraction angles (2θ): 7.6±0.2, 13.8±0.2, 20.6±0.2, 20.8±0.2, and 22.1±0.2.

In some embodiments, such a crystalline form exhibits an X-ray powder diffraction pattern comprising peaks at the following diffraction angles (2θ): 7.6±0.2, 13.8±0.2, 18.5±0.2, 19.0±0.2, 19.2±0.2, 19.7±0.2, 20.6±0.2, 20.8±0.2, 22.1±0.2, and 22.2±0.2.

In some embodiments, the crystalline form exhibits an X-ray powder diffraction pattern comprising some peaks selected from Table 21.

TABLE 21
XRPD Peak List of Form 3

| | Position (°2- | Intensity | |
|---|---|---|---|
| Peak | theta) | (Counts) | Rel. Int. (%) |
| 1 | 7.6 | 18615.0 | 100.0 |
| 2 | 13.8 | 5484.0 | 29.5 |
| 3 | 14.3 | 1912.0 | 10.3 |
| 4 | 15.2 | 2745.0 | 14.7 |
| 5 | 16.5 | 1954.0 | 10.5 |
| 6 | 18.5 | 3521.0 | 18.9 |
| 7 | 19.0 | 4847.0 | 26.0 |
| 8 | 19.2 | 3207.0 | 17.2 |
| 9 | 19.7 | 2971.0 | 16.0 |
| 10 | 20.1 | 1600.0 | 8.6 |
| 11 | 20.6 | 12599.0 | 67.7 |
| 12 | 20.8 | 7285.0 | 39.1 |
| 13 | 21.4 | 2015.0 | 10.8 |
| 14 | 21.7 | 1457.0 | 7.8 |
| 15 | 22.1 | 5720.0 | 30.7 |
| 16 | 22.2 | 3706.0 | 19.9 |
| 17 | 22.7 | 2181.0 | 11.7 |
| 18 | 25.0 | 1398.0 | 7.5 |
| 19 | 25.2 | 1294.0 | 7.0 |
| 20 | 25.9 | 1519.0 | 8.2 |
| 21 | 30.5 | 982.0 | 5.3 |
| 22 | 30.6 | 1023.0 | 5.5 |
| 23 | 37.2 | 953.0 | 5.1 |

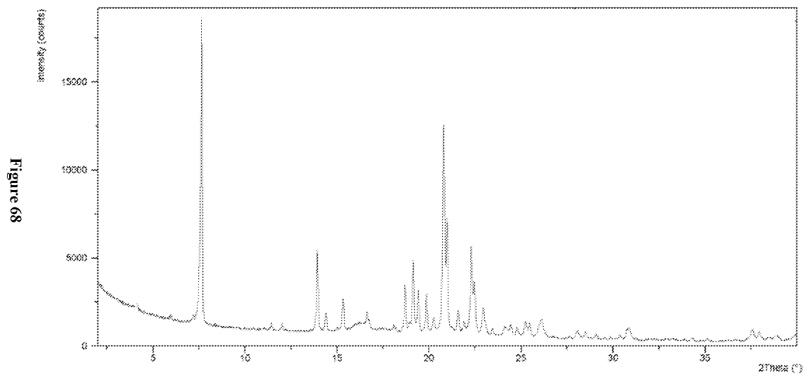

In some embodiments, the crystalline form exhibits an X-ray powder diffraction pattern substantially the same as depicted in FIG. 67.

### Form 4

In one aspect, a polymorph of the compound of Formula (I):

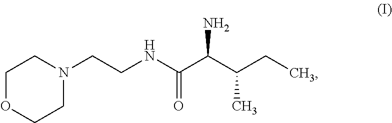 [SMILES: CC[C@H](C)[C@H](N)C(=O)NCCN1CCOCC1]

is provided and/or utilized in a crystalline form. In some embodiments, a particular crystalline form may be detected, for example in a composition, by detection of a set of characteristic peaks.

In some embodiments, such a crystalline form exhibits an X-ray powder diffraction pattern comprising peaks at the following diffraction angles (2θ): 5.2±0.2, 10.0±0.2, and 17.4±0.2.

In some embodiments, such a crystalline form exhibits an X-ray powder diffraction pattern comprising peaks at the following diffraction angles (2θ): 5.2±0.2, 10.0±0.2, 15.1±0.2, 17.4±0.2, and 22.6±0.2.

In some embodiments, such a crystalline form exhibits an X-ray powder diffraction pattern comprising peaks at the following diffraction angles (2θ): 5.2±0.2, 10.0±0.2, 10.4±0.2, 12.4±0.2, 15.1±0.2, 17.4±0.2, 20.7±0.2, 21.6±0.2, 22.1±0.2, and 22.6±0.2.

In some embodiments, the crystalline form exhibits an X-ray powder diffraction pattern comprising some peaks selected from Table 22.

TABLE 22
XRPD Peak List of Form 4

| | Position (°2- | Intensity | |
|---|---|---|---|
| Peak | theta) | (Counts) | Rel. Int. (%) |
| 1 | 5.2 | 10414.0 | 100.0 |
| 2 | 10.0 | 7885.0 | 75.7 |
| 3 | 10.4 | 1549.0 | 14.9 |
| 4 | 12.4 | 2231.0 | 21.4 |
| 5 | 15.1 | 4426.0 | 42.5 |
| 6 | 17.4 | 4589.0 | 44.1 |
| 7 | 18.9 | 1376.0 | 13.2 |
| 8 | 19.5 | 1501.0 | 14.4 |
| 9 | 20.2 | 1313.0 | 12.6 |
| 10 | 20.7 | 2696.0 | 25.9 |
| 11 | 21.6 | 2226.0 | 21.4 |
| 12 | 22.1 | 1891.0 | 18.2 |
| 13 | 22.6 | 3851.0 | 37.0 |
| 14 | 24.4 | 1081.0 | 10.4 |
| 15 | 24.7 | 1178.0 | 11.3 |
| 16 | 25.1 | 1473.0 | 14.1 |
| 17 | 25.5 | 1175.0 | 11.3 |
| 18 | 26.1 | 1141.0 | 11.0 |
| 19 | 26.4 | 997.0 | 9.6 |
| 20 | 28.5 | 976.0 | 9.4 |
| 21 | 29.1 | 990.0 | 9.5 |
| 22 | 38.3 | 1210.0 | 11.6 |

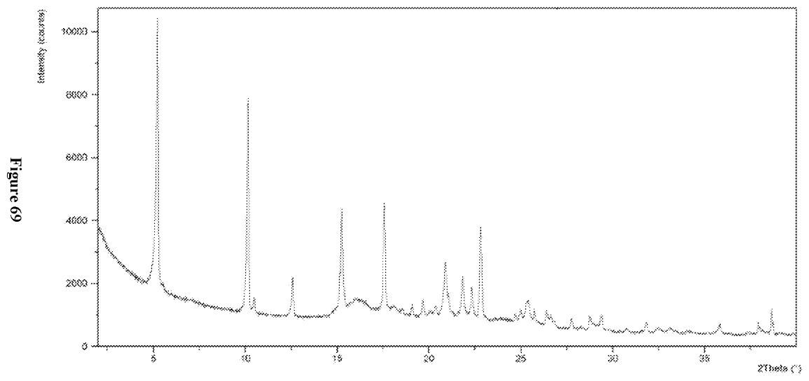

In some embodiments, the crystalline form exhibits an X-ray powder diffraction pattern substantially the same as depicted in FIG. 68.

### Synthesis

Procedures for making provided salts and crystalline forms, and preparations thereof, described herein are provided below. Starting materials used in the following schemes can be purchased or prepared by methods described in the chemical literature, or by adaptations thereof, using methods known by those skilled in the art in light of the present disclosure. The order in which the steps are performed can vary depending on the groups introduced and the reagents used, but would be apparent to those skilled in the art.

In some embodiments a procedure for compounds described herein may include one or more steps of recrystallizing compounds.

In one aspect, the disclosure provides a method of producing a crystalline form as disclosed herein, comprising: combining a compound of Formula (I):

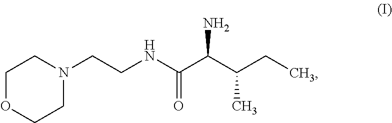 [SMILES: CC[C@H](C)[C@H](N)C(=O)NCCN1CCOCC1]

or a salt thereof, with a solvent, thereby forming a suspension or a solution thereof; optionally heating the suspension, thereby forming the solution; optionally cooling the solution; and forming a precipitate from the solution, thereby forming the crystalline form.

In some embodiments, the compound of Formula (I) is a salt thereof. In some embodiments, the method further comprises treating the suspension or the solution with a base.

In some embodiments, the method further comprises treating the suspension with a base. In some embodiments, the method further comprises treating the solution with a base. In some embodiments, the base is sodium hydroxide.

In some embodiments, the compound of Formula (I) is a free base. In some embodiments, the method further comprises treating the solution or the suspension with an acid. In some embodiments, the acid is adipic acid or gentisic acid. In some embodiments, the acid is adipic acid. In some embodiments, the acid is gentisic acid.

In some embodiments, the crystalline form is ADI Form A as disclosed herein.

In some embodiments, the solvent comprises tetrahydrofuran, ethyl acetate, ethanol, isopropyl alcohol, 2-methyltetrahydrofuran, or isopropyl acetate. In some embodiments, the solvent is tetrahydrofuran. In some embodiments, the solvent is ethyl acetate. In some embodiments, the solvent is ethanol. In some embodiments, the solvent is isopropyl alcohol. In some embodiments, the solvent is 2-methyltetrahydrofuran. In some embodiments, the solvent is isopropyl acetate.

In some embodiments, the solvent is 2-methyltetrahydrofuran or isopropyl acetate. In some embodiments, the solvent is 2-methyltetrahydrofuran. In some embodiments, the ratio of crystalline form to solvent is about 50 mg of the crystalline form to 0.5 mL of the solvent. In some embodiments, the suspension is heated from 25° C. to 80° C. In some embodiments, the suspension is cooled from 80° C. to 25° C.

The present disclosure also teaches that, in some embodiments, it may be desirable to store and/or utilize the crystalline form, e.g., ADI Form A, under low-humidity conditions, for example, in sealed vials and/or in containers and/or rooms with humidity control.

### Pharmaceutical Compositions

Pharmaceutical compositions, including and/or otherwise manufactured from provided salts, crystalline forms, and/or preparations thereof, may be prepared using a variety of technologies that are available for formulating a compound described in the present disclosure together with one or more physiologically acceptable carriers including excipients and auxiliaries which facilitate processing of the compound of Formula (I) into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure

In many embodiments, conventional pharmaceutical manufacturing technologies such as, for example, conventional mixing, dissolving, granulating, dragee-making, levigating, emulsifying, encapsulating, entrapping or compression processes may be utilized in the preparation of pharmaceutical compositions in accordance with the present disclosure.

Manufacturing of a pharmaceutical composition in accordance with the present disclosure (e.g., a pharmaceutical composition including a compound described herein (e.g., a compound of Formula (I) or a pharmaceutically acceptable salt or a crystalline form thereof)) may, in some embodiments, involve one or more steps of: mixing, dissolving, granulating, dragee-making, levigating, emulsifying, encapsulating, entrapping or compression processes.

Those skilled in the art, reading the present disclosure, will appreciate that it provides particular salts and crystal forms of a compound of Formula (I). Provided pharmaceutical compositions will include and/or be manufactured from/by way of at least one such salt or crystal form. Indeed, such skilled persons will appreciate that, in some embodiments, the present disclosure provides certain crystal forms of salt of a compound of Formula (I) that may be particularly useful for manufacturing pharmaceutical compositions.

Those skilled in the art, reading the present disclosure, will appreciate that it surprisingly provides salts of a compound of Formula (I), crystal forms, and preparations thereof, with particular characteristics (e.g., hygroscopicity characteristics) that are especially useful in manufacture and/or use of pharmaceutical compositions.

In some embodiments, provided salts, crystal forms, and/or preparations show particularly desirable stability characteristics. In some embodiments, the present disclosure provides preparations that have been stored and retain significant characteristics of the preparation prior to such storing.

In certain embodiments, pharmaceutical compositions provided herein may also include one or more preservatives to inhibit microbial activity. Suitable preservatives include quaternary ammonium compounds such as benzalkonium chloride, cetyltrimethylammonium bromide and cetylpyridinium chloride.

Pharmaceutical preparations for oral use can be obtained by mixing one or more solid excipients with one or more of salts or crystalline forms provided herein, optionally grinding the resulting mixture, and processing the mixture of granules, after adding suitable auxiliaries, if desired, to obtain tablets, pills, or capsules. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include, for example, push-fit capsules made of gelatin, as well as soft, sealed capsules made of gelatin and a plasticizer, such as glycerol or sorbitol. In some embodiments, push-fit capsules can contain the active ingredient(s) in admixture with filler such as lactose, binders such as starches, and/or lubricants such as talc or magnesium stearate and, optionally, stabilizers. In soft capsules, active compound(s) may be dissolved or suspended in suitable liquids, such as fatty oils, liquid paraffin, or liquid polyethylene glycols. In addition, stabilizers may be added.

In some embodiments, solid dosage forms described herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, provided pharmaceutical compositions may be in the form of a powder.

In some embodiments, pharmaceutical compositions described herein may be administered as a single dosage form or in multiple dosage forms (e.g., where multiple distinct compositions make up a single dose). In some embodiments, the pharmaceutical composition is administered in two, or three, or four, dosage forms.

In some embodiments, solid dosage forms, e.g., tablets, effervescent tablets, and capsules, are prepared by mixing particles of the compound described herein (e.g., a compound of Formula (I) or a pharmaceutically acceptable salt or a crystalline form thereof), with one or more pharmaceutical excipients to form a bulk blend composition. When referring to such bulk blend compositions as homogeneous, it is meant that the particles of a compound described herein (e.g., a compound of Formula (I) or a pharmaceutically acceptable salt or a crystalline form thereof), are dispersed evenly throughout the composition so that the composition may be subdivided into equally effective unit dosage forms, such as tablets, pills, and capsules. Individual dosage forms may also include film coatings, which disintegrate upon oral ingestion or upon contact with diluent. In some embodiments, such formulations can be manufactured by conventional pharmacological techniques.

In some embodiments the pharmaceutical composition may contain less than 10 wt % of excipients. In some embodiments the pharmaceutical composition may contain less than 5 wt % of excipients, such as less than 4 wt % of excipients, such as less than 3 wt % of excipients, such as less than 2 wt % of excipients. In certain embodiments, the pharmaceutical composition contains less than 2 wt % of excipients. In some embodiments, the pharmaceutical composition consists essentially of a compound described herein (e.g., a compound of Formula (I) or a pharmaceutically acceptable salt or a crystalline form thereof).

In some embodiments provided pharmaceutical compositions comprise from about 10 milligram (mg) (free base weight) to about 1,000 mg (free base weight) of a salt of the compound of Formula (I). In some embodiments provided pharmaceutical compositions comprise from about 10 mg (free base weight) to about 800 mg (free base weight) of a salt of the compound of Formula (I). The In some embodiments provided pharmaceutical compositions comprise from about 10 mg (free base weight) to about 600 mg (free base weight) of a salt of the compound of Formula (I). In some embodiments provided pharmaceutical compositions comprise from about 10 mg (free base weight) to about 500 mg (free base weight) of a salt of the compound of Formula (I).

As referred to herein, the “free base weight” refers to a calculated mass of the free base, i.e., the compound of Formula (I), based upon the mass of the salt in the composition. For example, to obtain a pharmaceutical composition with 200 mg (free base weight) of the compound of Formula (I), 260 mg of the ADI Form A 2:1, i.e., 1:0.5, free base/adipate salt of the compound of Formula (I) is added to the composition. The following equation may be used to calculate the weight of the compound of Formula (I) from the weight of the ADI Form A 2:1, i.e., 1:0.5, free base/adipate salt of the compound of Formula (I):

$$( weight ( g ) of the ADI Form A 2 : 1 free base / adipate salt of the compound of Formula ( I ) ) \div ( MW ( g / mol ) of the ADI Form A 2 : 1 free base / adipate salt of the compound of Formula ( I ) ) \times ( 2 mol free base / 1 mol salt ) \times ( MW of free base ) = weight of the free base$$

MW of the ADI Form A 2:1 free base/adipate salt of the compound of Formula (I): 632.84 g/mol
MW of free base (compound of Formula I): 243.35

As ADI Form A 2:1 free base/adipate salt of the compound of Formula (I) is also equivalent to having a 1:0.5 ratio, the weight of the compound of Formula (I) can also be calculated from:

$$( weight ( g ) of the ADI Form A 1 : 0.5 free base / adipate salt of the compound of Formula ( I ) ) \div ( MW ( g / mol ) of the ADI Form A 1 : 0.5 free base / adipate salt of the compound of Formula ( I ) ) \times ( 1 mol free base / 1 mol salt ) \times ( MW of free base ) = weight of the free base$$

MW of the ADI Form A 1:0.5 free base/adipate salt of the compound of Formula (I): 316.42 g/mol
MW of free base (compound of Formula I): 243.35

### Methods of Use

In one aspect, the present disclosure provides a method of treating a disease in a subject in need thereof, comprising administering to the subject a therapeutically effective amount of a salt or a crystalline form of a compound of Formula (I) as disclosed herein. In some embodiments, the disease is a disorder involving degradation, degeneration or dysfunction of cells expressing p75. In some embodiments, the disease is a nervous system disorder including (but not limited to) Alzheimer's disease, Parkinson's disease, Huntington's disease, tauopathies, neuroinflammatory disorders, stroke, traumatic brain injury, spinal cord injury, epilepsy, multiple sclerosis, amyotrophic lateral sclerosis, neuropathies, myopathies and various forms of retinal degeneration. In other embodiments, diseases might primarily lie outside of the nervous systems such as hair loss or immune disorders including various forms of arthritis.

In some embodiments, the disease is Alzheimer's Disease in a subject in need thereof. (e.g., mild to moderate, or preclinical stages such as Mild Cognitive Impairment (MCI) due to underlying Alzheimer's disease pathology).

Provided salts and crystal forms, and preparations and/or compositions manufactured from and/or including them, can be used to modulate p75 neurotrophin receptor activity, or for the treatment of diseases or conditions that would benefit, at least in part, from modulation of p75 neurotrophin receptor activity.

Thus, a method for treating any of the diseases or conditions described herein in a subject in need of such treatment, may involve (e.g., oral) administration of a pharmaceutical composition described herein, that is manufactured from and/or otherwise comprises a salt of Formula (I) or a crystal form thereof as provided by the present disclosure. Typically, such pharmaceutical compositions is administered in therapeutically effective amount(s) to the subject, and preferably in accordance with a dosing regimen that has been established to achieve a designated benefit when administered to an appropriate subject population (e.g., in a clinical trial).

Those skilled in the art, reading the present disclosure will appreciate that, in some embodiments, an appropriate subject may exhibit or be determined to exhibit mild cognitive impairment, which may be due to underlying Alzheimer's disease pathology, or mild to moderate Alzheimer's disease according to McKhann (2011) criteria, in some case with the application of certain diagnostic biomarkers e.g., within six months prior to the administration. In other cases, a subject may exhibit or be determined to be in preclinical or earlier clinical stages of the disease according to the results of appropriate biomarker testing.

In some embodiments, pharmaceutical compositions described herein can be administered for prophylactic and/or therapeutic treatments. In therapeutic applications, the compositions are administered to a patient already suffering from a disease or condition, in an amount effective to treat or at least partially arrest the symptoms or underlying degeneration and progression of the disease or condition. Those skilled in the art will appreciate that an effective amount for such use may depend on the severity and course of the disease or condition, previous therapy, the patient's health status, weight, and response to the drugs, and the judgment of the treating physician.

In prophylactic applications, including those intended to delay onset of clinical symptoms such as loss of cognitive function, provided pharmaceutical compositions are administered to a patient susceptible to or otherwise at risk of a particular disease, disorder or condition. This status can be defined, for example, by the application of available genetic and/or biomarker testing. Such an amount is defined to be a “prophylactically effective amount or dose.” Those skilled in the art will appreciate that the precise amounts for prophylaxis may also depend on the patient's state of health, weight, and the like, and furthermore that effective amounts for this use may depend on the severity and course of the disease, disorder or condition, previous therapy, the patient's health status and response to the drugs, and the judgment of the treating physician.

In some embodiments of any one of the relevant methods described herein, the method described may be used to treat or prevent Alzheimer's disease in subjects with particular characteristics. For example, methods of the disclosure may be used to treat subjects who exhibit mild to moderate Alzheimer's disease according to McKhann (2011) criteria within six months prior to said administration. Other subjects might be in earlier stages such as preclinical or prodromal stages as determined by genetic and/or biomarker testing, for example, in patents having a level (e.g., a plasma level) of tau protein phosphorylated at, e.g., p-tau217, p-tau243, p-tau181, and/or p-tau231, that is indicative of the presence of Alzheimer's Disease pathology in the brain. Methods of the disclosure may be used to treat, delay onset or prevent Alzheimer's disease in subjects from 50 to 100 years old, such as from 50 to 90 years old, such as from 65 to 90 years old. Methods of the disclosure may be used to treat, delay onset, manage, or prevent Alzheimer's disease in subjects exhibiting or determined to exhibit an E4 allele of apolipoprotein E (ApoE) gene (ApoE4). Methods of the disclosure may be used to treat, manage, or prevent Alzheimer's disease in subjects not exhibiting or determined to not exhibit an ε4 allele of apolipoprotein E (ApoE) gene (ApoE4).

In some embodiments of any one of the relevant methods described herein, the method described may provide or result in a slowing of deterioration of, in an improvement of, a value of one or more Alzheimer's disease metrics relative to a baseline value measured at or within six months (e.g., within three months) preceding said administration. The Alzheimer's disease metrics may be selected from anatomical or statistical regional or voxel-based brain glucose metabolism (¹⁸F-FDG-PET) rate, magnetic resonance imaging (MRI) structural or voxel-based or volumetric imaging, cerebrospinal Alzheimer's disease-relevant biomarker levels, blood or plasma biomarkers. CSF biomarkers might include those related to synaptic degeneration such as SNAP-25 or neurogranin; those related to inflammation such as YKL-40 or those related to tau pathology such as total tau or p-tau217. Plasma biomarkers might include p-tau217 or GFAP. The Alzheimer's disease metrics may also be selected from performance on the cognitive testing methods such as ADAS-cog, MMSE and other cognitive testing approaches.

In some embodiments of any one of the relevant methods described herein, methods of the disclosure reduce or prevent loss of certain brain region volumes in said subject. In some embodiments, the brain region volume comprises one or more of a hippocampus volume, basal forebrain volume, lingual gyrus volume, parahippocampal gyrus, and orbitofrontal cortex, parietal cortex and cingulate cortex in said subject. In some embodiments volume of lateral ventricles in measured as an indicator of general brain volume; increased size of lateral ventricular volume is an indicator of diffuse loss of brain parenchymal volume. In some embodiments, the brain region volume comprises other brain region volumes in said subject. Such volumes could be derived from whole brain or targeted region voxel wise or voxel-based analyses.

In some embodiments of any one of the relevant methods described herein, methods of the disclosure reduce or prevent loss of hippocampus volume or loss or changes in other brain region or voxel-based volumes in said subject. In certain embodiments, methods of the disclosure reduce or prevent or mitigate an increase in certain species of amyloid beta (Aβ) level such as Aβ42 (e.g., in a bodily fluid, such as a cerebrospinal spinal fluid or blood). In certain embodiments, methods of the disclosure lead to a reduction in fluid levels of Aβ42, 40 or other species. In certain embodiments, methods of the disclosure reduce or prevent or mitigate an increase in tau or modified versions of tau level (e.g., in a bodily fluid, such as a cerebrospinal spinal fluid or blood or plasma).

In some embodiments, a method of the present disclosure delays or prevents the progression of Alzheimer's disease in the subject. In some embodiments, the delaying or preventing progression comprises one or more of ameliorating, delaying, or preventing symptoms of Alzheimer's Disease compared to a subject not receiving the p75^{NTR} receptor modulator. In some embodiments, a subject not receiving the p75^{NTR} receptor modulator receives a placebo or no treatment. In some embodiments, the subject exhibits one or both of improved cognition or improved function compared to a patient receiving no therapy or a placebo therapy. In some embodiments, the symptoms are assessed by neurological testing. In some embodiments, the neurological testing is selected from the Alzheimer's Disease Assessment Scale-Cognitive (ADAS-Cog), the Mini Mental Status Exam (MMSE), the Geriatric Depression Scale (GDS), and combinations thereof. In some embodiments, the neurological testing is the Alzheimer's Disease Assessment Scale-Cognitive (ADAS-Cog). In some embodiments, the ADAS-Cog score is ADAS11 or ADAS13. In some embodiments, the ADAS-Cog score is ADAS11. In some embodiments, the ADAS-Cog score is ADAS13. In some embodiments, the neurological testing is the Clinical Dementia Rating-Sum of Boxes (CDR-SB). In some embodiments, the neurological testing is the Alzheimer's Disease Composite Score (ADCOMS). In some embodiments, the corresponding reference is determined from an untreated control. In some embodiments, the corresponding reference change is a corresponding pretreatment change within a corresponding time period in said subject. In some embodiments, the time period is about three months. In some embodiments, the time period is about six months. In some embodiments, the period of time is about 18 months.

In some embodiments of any one of the relevant methods described herein, the cognitive testing methods include one or more of the Alzheimer's disease assessment scale (ADAS-cog), mini mental status exam (MMSE), the Geriatric Depression Scale (GDS), and other tests. In some embodiments, the cognitive testing methods include one or more of ADAS-cog and MMSE. In some embodiments, the cognitive testing method is selected from any one or any subset of those set forth in Table 23.

TABLE 23
List of cognitive testing methods

Alzheimer's disease assessment scale (ADAS-cog)
Mini Mental Status Exam (MMSE)
Category fluency test

In some embodiments of any one of the relevant methods described herein, the bodily fluid is a cerebrospinal spinal fluid, blood, serum, or plasma. In some embodiments, the bodily fluid is a cerebrospinal spinal fluid. In some embodiments, the bodily fluid is blood. In some embodiments, the bodily fluid is serum. In some embodiments, the bodily fluid is plasma.

In some embodiments of any one of the relevant methods described herein, the corresponding reference (e.g., level, value, rate, etc.) is a corresponding pretreatment level, value, rate (etc.) of the subject (e.g., in said bodily fluid). In some embodiments, the corresponding reference (e.g., level, value, rate, etc.) is determined from an untreated control (e.g., a corresponding subject or a placebo cohort).

In some embodiments of any one of the relevant methods described herein, the duration or time period (over which a change or a rate of change, for example, an increase or a rate of increase, in the one or more neurodegeneration biomarkers or indicators is determined) is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months. In some embodiments, the duration or time period is at least about 5 days. In some embodiments, the duration or time period is at least about 10 days. In some embodiments, the duration or time period is at least about 15 days. In some embodiments, the duration or time period is at least about 20 days. In some embodiments, the duration or time period is at least about 25 days. In some embodiments, the duration or time period is at least about 30 days. In some embodiments, the duration or time period is at least about 1 month. In some embodiments, the duration or time period is at least about 2 months. In some embodiments, the duration or time period is at least about 3 months. In some embodiments, the duration or time period is at least about 4 months. In some embodiments, the duration or time period is at least about 5 months. In some embodiments, the duration or time period is at least about 6 months. In some embodiments, the duration or time period is at least about 7 months In some embodiments, the duration or time period is at least about 8 months In some embodiments, the duration or time period is at least about 9 months In some embodiments, the duration or time period is at least about 10 months In some embodiments, the duration or time period is at least about 11 months In some embodiments, the duration or time period is at least about 12 months In some embodiments, the duration or time period is at least about 13 months In some embodiments, the duration or time period is at least about 14 months In some embodiments, the duration or time period is at least about 15 months In some embodiments, the duration or time period is at least about 16 months In some embodiments, the duration or time period is at least about 17 months. In some embodiments, the duration or time period is at least about 18 months.

In some embodiments of any one of the relevant methods described herein, the duration or time period (over which a change or a rate of change, for example, an increase or a rate of increase, in the one or more neurodegeneration biomarkers or indicators is determined) is from about 5 days to about 6 months, from about 10 days to about 6 months, from about 15 days to about 6 months, from about 20 days to about 6 months, from about 25 days to about 6 months, from about 30 days to about 6 months, from about 1 month to about 6 months, from about 2 months to about 6 months, from about 3 months to about 6 months, from about 4 months to about 6 months, or from about 5 months to about 6 months. In some embodiments, the duration or time period is from about 5 days to about 6 months. In some embodiments, the duration or time period is from about 10 days to about 6 months. In some embodiments, the duration or time period is from about 15 days to about 6 months. In some embodiments, the duration or time period is from about 20 days to about 6 months. In some embodiments, the duration or time period is from about 25 days to about 6 months. In some embodiments, the duration or time period is from about 30 days to about 6 months. In some embodiments, the duration or time period is from about 1 month to about 6 months. In some embodiments, the duration or time period is from about 2 months to about 6 months. In some embodiments, the duration or time period is from about 3 months to about 6 months. In some embodiments, the duration or time period is from about 4 months to about 6 months. In some embodiments, the duration or time period is from about 5 months to about 6 months. In some embodiments, the duration or time period is at least about 18 months.

In some embodiments of any one of the relevant methods described herein, the duration or time period (over which a change or a rate of change, for example, an increase or a rate of increase, in the one or more neurodegeneration biomarkers or indicators is determined) is from about 5 days to about 18 months, from about 10 days to about 18 months, from about 15 days to about 18 months, from about 20 days to about 18 months, from about 25 days to about 18 months, from about 30 days to about 18 months, from about 1 month to about 18 months, from about 2 months to about 18 months, from about 3 months to about 18 months, from about 4 months to about 18 months, or from about 5 months to about 18 months. In some embodiments, the duration or time period is from about 5 days to about 18 months. In some embodiments, the duration or time period is from about 10 days to about 18 months. In some embodiments, the duration or time period is from about 15 days to about 18 months. In some embodiments, the duration or time period is from about 20 days to about 18 months. In some embodiments, the duration or time period is from about 25 days to about 18 months. In some embodiments, the duration or time period is from about 30 days to about 18 months. In some embodiments, the duration or time period is from about 1 month to about 18 months. In some embodiments, the duration or time period is from about 2 months to about 18 months. In some embodiments, the duration or time period is from about 3 months to about 18 months. In some embodiments, the duration or time period is from about 4 months to about 18 months. In some embodiments, the duration or time period is from about 5 months to about 18 months. In some embodiments, the duration or time period is from about 6 months to about 18 months. In some embodiments, the duration or time period is from about 7 months to about 18 months. In some embodiments, the duration or time period is from about 8 months to about 18 months. In some embodiments, the duration or time period is from about 9 months to about 18 months. In some embodiments, the duration or time period is from about 10 months to about 18 months. In some embodiments, the duration or time period is from about 11 months to about 18 months. In some embodiments, the duration or time period is from about 12 months to about 18 months. In some embodiments, the duration or time period is from about 13 months to about 18 months. In some embodiments, the duration or time period is from about 14 months to about 18 months. In some embodiments, the duration or time period is from about 15 months to about 18 months. In some embodiments, the duration or time period is from about 16 months to about 18 months. In some embodiments, the duration or time period is from about 17 months to about 18 months.

In some embodiments of any one of the relevant methods described herein, the subject exhibits or is determined to exhibit mild to moderate Alzheimer's disease according to McKhann (2011) criteria within six months prior to said administration. In some embodiments, the subject exhibits or is determined to exhibit mild to moderate Alzheimer's disease according to McKhann (2011) criteria within three months prior to said administration. In some embodiments, the subject exhibits or is determined to be at high risk or in a pre-clinical or prodromal state of Alzheimer's disease as determined by genetic risk and/or biomarkers within six months prior to said administration. In some embodiments, the subject exhibits or is determined to be at high risk or in a pre-clinical or prodromal state of Alzheimer's disease as determined by genetic risk and/or biomarkers within three months prior to said administration. In some embodiments, the subject exhibits or is determined to exhibit an ε4 allele of apolipoprotein E (ApoE) gene. In some embodiments, the subject exhibits or is determined to exhibit no ε4 allele of apolipoprotein E (ApoE) gene. In some embodiments, the subject is between the ages of 50 and 90. In some embodiments, the subject is younger than about 72 years of age. In some embodiments, the subject is of about 72 years of age or older.

The disclosure provides a method of treating Alzheimer's Disease in a subject comprising administering an effective amount of a provided salt in some embodiments wherein the method delays or prevents progression of Alzheimer's Disease in the subject.

In another aspect, the disclosure provides a method of treating Alzheimer's Disease in a subject comprising administering an effective amount of a crystalline form in some embodiments wherein the method delays or prevents progression of Alzheimer's Disease in the subject.

In some embodiments, the delaying or preventing progression comprises one or more of ameliorating, delaying, or preventing symptoms of Alzheimer's Disease compared to an untreated subject.

In some embodiments, symptoms are assessed by neurological testing selected from the group consisting of: the Alzheimer's Disease Assessment Scale-Cognitive (ADAS-Cog), the Mini Mental Status Exam (MMSE), the Geriatric Depression Scale (GDS), and combinations thereof.

In some embodiments, symptoms are assessed by Mini Mental Status Exam (MMSE).

### EXAMPLES

Procedures disclosed herein can be conducted in a number of ways based on the teachings contained herein and procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

### Abbreviations

### General

ADAS-cog Alzheimer's disease assessment scale-cognitive subscale
ADCS-iADL Alzheimer's disease cooperative study-instrumental activities of daily living for mild cognitive impairment
ADI adipic salt of the compound of Formula (I)
ALS amyotrophic lateral sclerosis
APC annual percent change
API advanced pharmaceutical ingredient
ApoE apolipoprotein
ApoE4 apolipoprotein gene
aq aqueous
aq. aqueous
Aβ amyloid beta
CHI3L1 chitinase 3-like 1
conc concentration
conc. concentration
DSC differential scanning calorimetry
DVS dynamic vapor sorption
EAFUS substances added to food
Eq equivalents
Et ethyl
FDG-PET fluoro-deoxy-glucose positron emission tomography
GDS geriatric depression scale
GEN gentisic salt of the compound of Formula (I)
GRAS generally regarded as safe
h hour or hours
HPLC high-performance liquid chromatography
KF Karl Fischer water content
mAU milliabsorbance unit
Me methyl
MHz megahertz
min minute
MMSE mini-mental state examination
MRI magnetic resonance imaging
MS mass spectrometry
MSZ metastable zone
MUC mucic salt of the compound of Formula (I)
NA not applicable
NG neurogranin
NG36 neurogranin 36
NMR nuclear magnetic resonance
NP not performed
ø diameter
ORO orotic salt of the compound of Formula (I)
Ph phenyl
ppm parts per million
RH relative humidity
RT room temperature
SAF substances added to food
sec second or seconds
SNAP25 synaptosome associated protein 25
SNAP-25 synaptosome associated protein 25
SSQ sum of squares
sTREM2 soluble triggering receptor expressed on myeloid cells 2
SYT1 synaptotagmin 1
TAR tartaric salt of the compound of Formula (I)
TAR thermal analysis
TGA thermogravimetric analysis
v volume
vol volume
WSSQ weighted sum of squares
wt weight
XRPD x-ray powder diffraction
YKL40 chitinase 3-like 1

### Solvents, Reagents and Conditions

2-MXB 2-methoxybenzoic
3-HXB 3-hydroxybenzoic
3-MXB 3-methoxybenzoic
AcOH acetic acid
BHS bishydrogen sulfate
CSA camphorsulfonic acid
DCM dichloromethane
DMSO dimethylsulfoxide
EtOAc ethyl acetate
EtOH ethanol
HPMC hydroxypropyl methylcellulose
IPA isopropyl alcohol
MTBE methyl tert-butyl ether
MeCN acetonitrile
MEK methyl ethyl ketone
MIK methyl isobutyl ketone
NaOMe sodium methoxide
RT room temperature
TBME methyl tert-butyl ether
THF tetrahydrofuran

### General Experimental

### Analytical Instrumentation

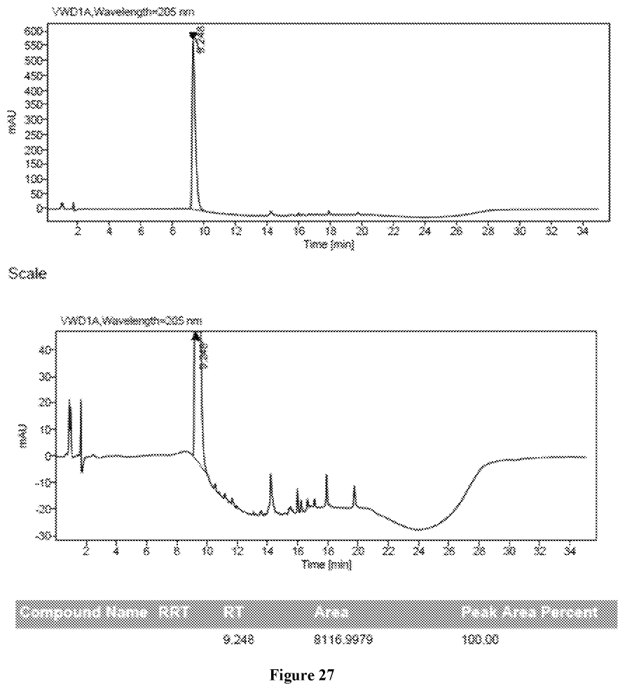

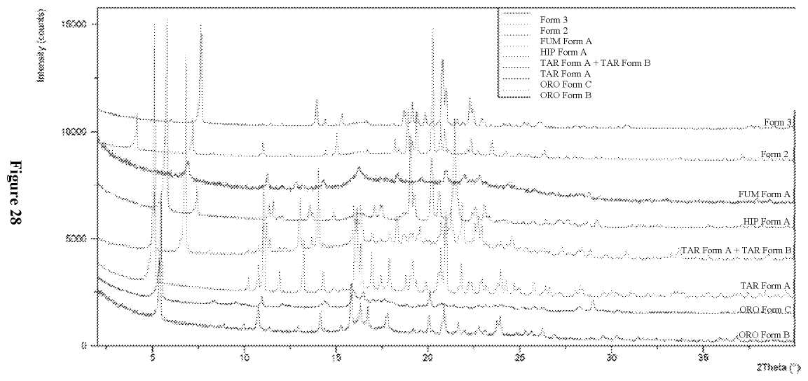

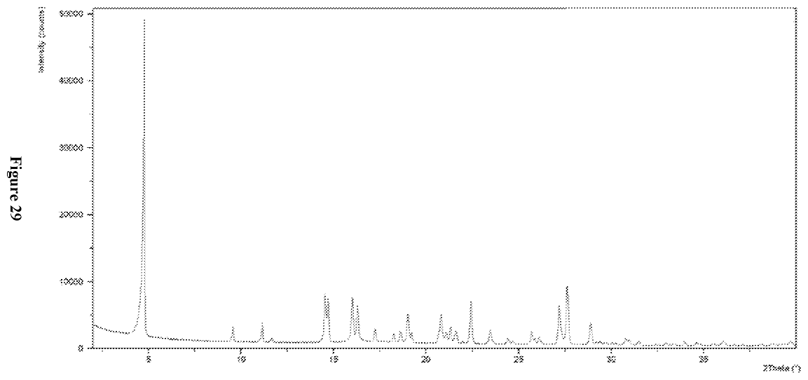

X-Ray Powder Diffraction (XRPD). Diffraction data was collected using a PANanalytical EMPYREAN vs. 8.2 20201404 diffractometer with a Cu-Kα radiation (λ=1.541874 Å) source operating at 45 kV and 40 mA in transmission theta-theta configuration. Samples were scanned from 2 to 40°2theta with a step size of 0.013°2theta and a time per step of 78.795 sec. Data collection was performed with DATA COLLECTOR vs. 7.1.
Differential Scanning Calorimetry (DSC). DSC analyses were performed using a Thermal Analysis (TA) Discovery instrument model DSC 25. The samples, typically weighing 1-3 mg, were heated in a sealed standard Tzero aluminum pan at a heating rate of 10° C./min under a nitrogen glass flow of 50 ml/min. A sealed empty Tzero aluminum pan was used as a reference. Data collection was performed with TRIOS software.
Thermogravimetric Analysis (TGA) TGA analyses were conducted in a Thermal Analysis (TA) Discovery instrument model TGA 550 using a nitrogen atmosphere with a gas flow of 60 mL/min and a heating rate of 10° C./min. Approximately 1 to 5 mg of sample was used. Data collection was performed with TRIOS software.
HPLC. HPLC analyses were performed on an Agilent 1220 Infinity II Liquid Chromatography system, equipped with a high-pressure binary pump, an auto-injector, a thermostatically controlled column compartment and a variable wavelength detector. For data processing and acquisition, OpenLab CDS software was used. Samples were weighed in glass vials and filtered prior to injection in the HPLC system. Table 24 and Table 25 describe the HPLC method conditions used to determine the purity and concentration of the salts. Using this method, the compound of Formula (I) peak elutes at approximately 9.2 min. The compound of Formula (I) was analyzed by HPLC as a reference (see FIG. 27).

TABLE 24
HPLC Parameters

| Column | XBridge Shield RP18, 3.5 μm, 4.6 x | |
|---|---|---|
| Column temperature | 30° | C. |
| Flow rate | 1 | mL/min |
| Mobile phase A | H₂O pH 10 with NH₄OH | |
| Mobile phase B | MeCN | |
| Injection volume | 15 | μL |
| Detection wavelength | 205 | nm |

| | |
|---|---|
| Sample Preparation | 0.5 mg/mL in MeCN:H₂O (1:1) |

TABLE 25
HPLC Method

| Time (min) | % A | % B |
|---|---|---|
| 0.5 | 90 | 10 |
| 5.5 | 90 | 10 |
| 15.5 | 10 | 90 |
| 18.5 | 10 | 90 |
| 24.5 | 90 | 10 |

Balances. A RAGWAG analytical balance model AS.220 R2 PLUS and a TA microbalance from TA Discovery TGA 550 instruments were used.
Proton Nuclear Magnetic Resonance (¹H NMR). ¹H NMR analyses were performed using an Agilent Mercury400 NMR Spectrometer. Approximately 5 mg of the sample was dissolved in 0.5-0-7 mL of deuterated DMSO.
Heating And Cooling Blocks. Temperature controlled experiments were performed with a Polar Bear Plus heating and cooling platform from Cambridge Reactor Design.
Humidity Chambers. The temperature was maintained using IVYX Scientific incubators, and the humidity by using a saturated aqueous sodium chloride solution for 75% RH/40° C. and a saturated aqueous potassium sulfate solution for 96% RH/25° C.
Particle Size Distribution (PSD) Analysis. Data were collected on a Malvern Mastersizer 3000 with red (4 mW He—Ne, 632.8 nm) and blue (10 mW LED, 470 nm) lasers. For wet measurements, the Hydro MV dispersion unit was used covering the range 10 nm to 3.5 mm. Instrument control and data analysis was performed using Mastersizer 3000 software.

### Particle Size Distribution Parameters

Dispersion unit Hydro MV
Dispersant Heptane with 0.2 vol % Span 80
Stir speed (rpm) 2500
Sample preparation ~3 ml dispersant added to the sample (mass ~22 mg), sample swirled and whole sample added to the sizer
Pre-measurement delay (sec) 100
Measurement time (sec) 20 (10 for red laser then 10 for blue laser)
Optical Model Non-spherical particle type (Mie theory)
Optical Properties Refractive Index: 1.53; Absorption Index: 0.00
Analysis 3 samples analysed 3 times consecutively
Reporting The 9 sets of raw data were averaged to give the Global Mean

### Buffer Solution Preparation

pH 1.2 (HCl—KCl): Potassium chloride (149.1 mg) was dissolved in deionized water (10 mL). 2.5 mL of the KCl solution were added into a 10 mL graduated measuring cylinder and 0.2 M HCl (aq.) (4.3 mL) were added. The pH was adjusted to pH 1.2 with 1M HCl and deionized water was added to volume.
pH 4.5 (Acetic acid-Sodium acetate): Sodium acetate (18 mg) was dissolved in deionized water (8 ml). 2M acetic acid solution (140 μL) were added, the pH was adjusted to pH 4.5 with 1M NaOH or 1M HCl and deionized water was added to volume.
pH 6.5 (NaOH—KH₂PO₄): Potassium phosphate monobasic (272 mg) was dissolved in deionized water (10 mL). 2.5 mL of the KH₂PO₄ solution were added into a 10 mL graduated measuring cylinder and 0.2 M NaOH (aq.) (700 μL) were added. The pH was adjusted to pH 6.5 with 1M NaOH and deionized water was added to volume.
pH 7.4 (NaOH—KH₂PO₄): Potassium phosphate monobasic (272 mg) was dissolved in deionized water (10 mL). 2.5 mL of the KH₂PO₄ solution were added into a 10 mL graduated measuring cylinder and 0.2 M NaOH (aq.) (2.0 mL) were added. The pH was adjusted to pH 7.4 with 1M NaOH and deionized water was added to volume.

### Example 1: Preparation and Characterization of the Compound of Formula (I)

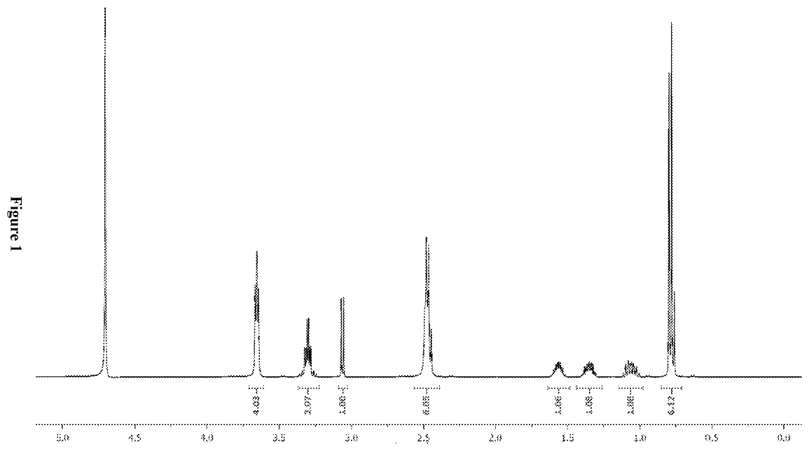

The BHS salt of the compound of Formula (I) (1 g) was suspended in DCM (20 V, 20 mL/g). An aqueous solution of NaOH 1M (9.2 mL, 4 equivalents) was added forming two layers. The mixture was transferred to a separatory funnel, was shaken, the phases were allowed to separate and the organic layer (lower) was drained. The aqueous layer (upper) was extracted with DCM (3×20 V, 20 mL) and the organic layers were combined and dried over sodium sulfate. The solution was concentrated to dryness in the rotary evaporator to give an oil (555 mg, 98%). The oil was analyzed by ¹H NMR and it is consistent with the structure (see FIG. 1).

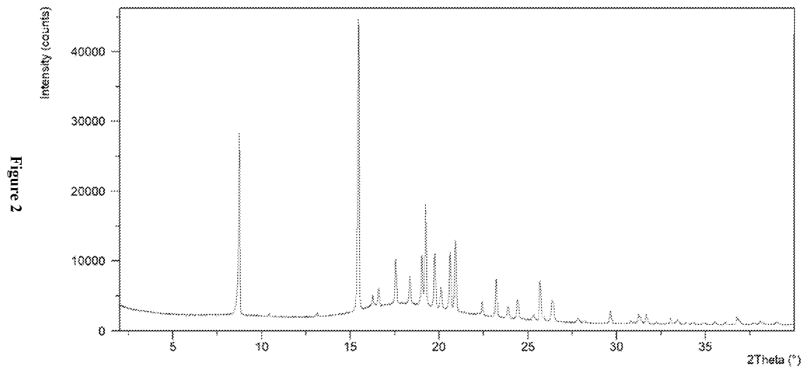

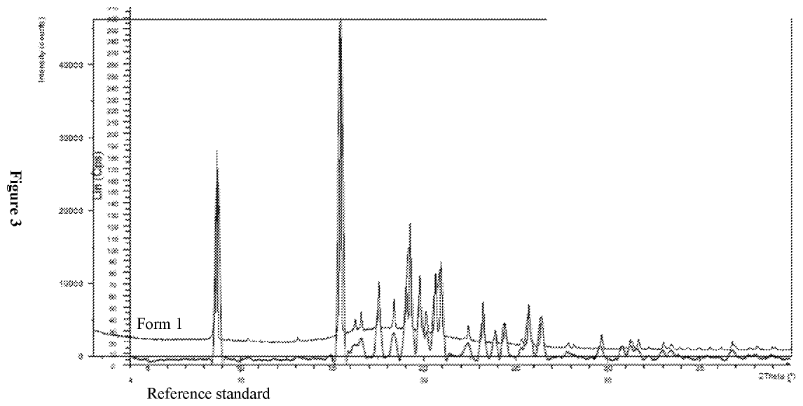

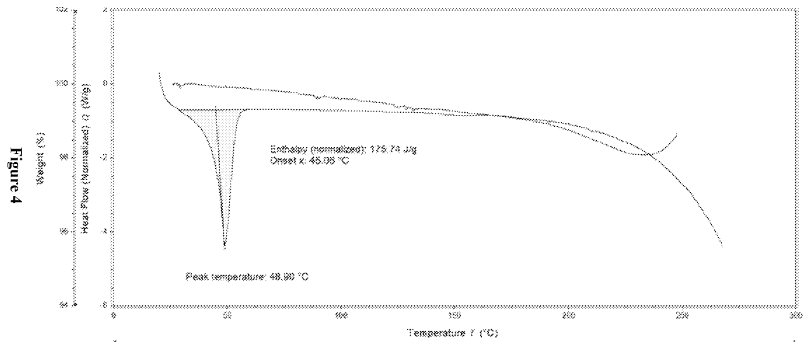

The compound of Formula (I) (oil, 1 g) was dissolved in IPA (10 mL) and evaporated in the rotary evaporator to dryness. Residual solvent was evaporated with nitrogen flow and a white solid was obtained in the round-bottomed flask. The resulting crystalline solid was analyzed by XRPD, DSC and TGA (see FIG. 2 and FIG. 4) and an XRPD comparison is showed in FIG. 3. The crystalline free base is anhydrous (no significant weight loss in TGA), with low melting point (onset 45° C. by DSC). The results obtained for this free-base crystalline form (Form 1) of the compound of Formula (I) are consistent with a reference standard.

### Example 2: Salt/Cocrystal Screening

The compound of Formula (I) was previously evaluated to identify salts and solid forms to be used in certain clinical trials associated with LM11A-31. Previously, the sulfate form of the compound of Formula (I) was selected, and described as having desirable stability. See in WO2011060262. However, during process development of this sulfate form of LM11A-31, it was surprisingly discovered that the sulfate form suffered from hygroscopicity and stability liabilities in scale-up.

### Summary

New salt and crystal form screening was performed on the compound of Formula (I). The present Example reports the process and outcome of this screening, namely that numerous (at least 59) coformers were analyzed across at least 184 screening conditions, and only eleven salts and 3 new polymorphs of the free base were identified.

Of those new salt and crystal forms, it was surprisingly discovered that the gentisate, orotate and hemi-adipate salts provide favorable stability and hygroscopicity characteristics in comparison with the sulfate salt as benchmark. These three salts showed high solubility (>10 mg/mL) over the pH range 1.2 to 7.4. Polymorphism assessments showed that the orotate and the gentisate salts exhibits polymorphism, while only one form was seen for the hemi-adipate salt.

Process development, solubility modelling and scale-up were performed for the gentisate and hemi-adipate salts. Three batches of 80-100 g of gentisate and two batches of 140-150 g of the hemi-adipate salt were prepared and characterized.

### Salt Screen

Those skilled in the art are aware that the standard set of pharmaceutically acceptable salts includes acetate, ascorbate, bicarbonate, calcium, chloride, citrate, gluconate, lactate, magnesium, malate, phosphate, potassium, sodium, succinate, sulfate, and tartrate. See, for example, Dhaval et al., Pharmaceutical Salts: Comprehensive Insights from Fundamental Chemistry to FDA Approvals (2019-2023) AAPS Phar Sci Tech 26:36, Jan. 16, 2025.

In light of the discovery of hygroscopicity and stability challenges with a previously-selected sulfate salt, a new salt screen was designed that included unorthodox strategies.

The compound of Formula (I) contains two basic sites with pKas of 6.9 and 8.4. The molecule also contains both hydrogen bond donors (NH) and acceptors (amine) and may form cocrystals with suitable coformers.

Based on the pKa of the API and on the potential binding motifs analysis, 59 coformers were selected as amongst pharmaceutically acceptable acids and compounds present in the GRAS (Generally Regarded As Safe) or SAF (Substances Added to Food, formerly EAFUS) lists.

The coformers, solvents and techniques used in the salt screen are summarized in Table 26. As can be seen, these include a variety of agents not typically found in conventional salt screens.

TABLE 26
Coformers, Solvents and Techniques Selected for Salt Screen
Coformers

| | | |
|---|---|---|
| 2,4,6-Trimethylphenol | Trimethylamine N-oxide | Sorbic Acid |
| 2,4-Dihydroxybenzoic Acid | Urea | L-Tartaric acid |
| 2,4-Lutidine | Vanillin | Fumaric acid |
| 2,5-Dihydroxybenzoic Acid | Ketoglutaric acid | L-Ascorbic acid |
| 2,6-Lutidine | Glucuronic acid | L-Lactic acid |
| Acetic Acid | Citric acid | Octanoic acid |
| Benzenesulfonic Acid | L-Tartaric acid | L-Glutamic acid |
| Betaine | Aspartic acid | Glutaric Acid |
| Ethyl Vanillin | Benzoic acid | p-Toluenesulfonic acid |
| Hippuric Acid | L-Malic acid | Methanesulfonic acid |
| Maleic Acid | Mucic acid | 2,3,6-Trimethylphenol |
| m-Cresol | Phosphoric acid | 4-Allyl-2,6-dimethoxyphenol |
| Nicotinamide | Adipic Acid | 4-tert-Butylphenol |
| Nicotinic Acid | Benzoic acid | Hydrochloric acid |
| Orotic Acid | Citric acid | 2-Methoxybenzoic Acid |
| 4-Aminobenzoic Acid | Linoleic Acid | 3-Methoxybenzoic Acid |
| p-Cresol | DL-Malic acid | 4-Methoxybenzoic Acid |
| Saccharine | L-Malic acid | 3,4-Dihydroxybenzoic Acid |
| Succinic Acid | Propionic acid | 3-Hydroxybenzoic Acid |
| DL-Tartaric acid | D-Tartaric acid | |

Solvents

| | |
|---|---|
| IPA | EtOAc |

Techniques

| | | |
|---|---|---|
| Heat-Cool cycling | Slurries at 0° C. | Slow evaporation |
| Wet grinding | | |

All solids from the screening experiments were analysed by XRPD before and after drying the sample under vacuum at RT overnight. The resulting patterns were compared with the corresponding coformer. Those samples showing a distinct XRPD pattern were further characterized by some, or all, of the following techniques, depending on material availability: ¹H NMR, DSC and TGA. Then, they were stored in XRPD sample holders in a humidity chamber at 40° C./75% RH for one week. Upon storage, samples were monitored by XRPD over time.

An additional stability study was performed at 25° C./96% RH for one week with the salts considered most promising for stability reasons. Samples were also monitored by XRPD over time.

The results are provided in Table 27, Table 28, Table 29, and Table 30.

### Heat-Cool Cycling

Dried compound of Formula (I) (approx. 20 mg) and 1.1 equivalents of the corresponding acid were weighed into 2 mL HPLC vials. The corresponding solvent (100 μL) was added and the mixtures were subjected to heat-cool cycles between 25° C. and 50° C. (30 min at each temperature, with heating and cooling at 1° C./min) for 1 to 4 days. The suspensions were centrifuged, decanted and solids were analysed by XRPD before and after drying under vacuum at RT.

Slurries at Low Temperature after Heat-Cool Cycling

Oils and solutions from the heat-cool cycling experiments were stirred at 0° C. for 24 h. However, no solids were observed in any of the experiments.

### Slow Evaporation of Samples at Low Temperature

Solutions of samples at low temperature were allowed to slowly evaporate until dryness at room temperature; via a needle in a septum, or directly opening the vial for the low volatile solvents. However, oils were observed in all the experiments.

### Wet Grinding

The compound of Formula (I) Form 1 (20 mg), 0.5-2.01 equivalents of the corresponding coformer and the corresponding solvent (5-10 μL) were weighed into 2 mL HPLC vials, each one equipped with 3 stainless steel 3 mm ø balls and were ground in a Fritsch Pulverisette planetary mill at 400 rpm for a period of 16 cycles of 1 h with 5 minutes resting between cycles. The resulting solids were analysed by XRPD before and after drying under vacuum at RT unless oils or solutions were obtained.

### Results—Screening

All results from the salt/cocrystal screening study are provided in Table 27 below.

TABLE 27
All Salt/Cocrystal Screen Experiments

| | | | | Heat-Cool Cycling | | Wet Grinding* | |
|---|---|---|---|---|---|---|---|
| Ref. | Coformer | Eq. | Solvent | Wet | Dry | Wet | Dry |
| 1 | 2,4- | 1.1 | IPA | Solution | NP | NP | NP |
| | Dihydroxybenzoic | | | | | | |
| | acid | | | | | | |
| 2 | 2,4- | 2 | IPA | Solution | NP | NP | NP |
| | Dihydroxybenzoic | | | | | | |
| | acid | | | | | | |
| 3 | 2,4- | 1.1 | EtOAc | Solution | NP | NP | NP |
| | Dihydroxybenzoic | | | | | | |
| | acid | | | | | | |
| 4 | 2,4- | 2 | EtOAc | Solution | NP | NP | NP |
| | Dihydroxybenzoic | | | | | | |
| | acid | | | | | | |
| 5 | 2,4- | 1 | EtOAc | NP | NP | Coformer | NP |
| | Dihydroxybenzoic | | | | | | |
| | acid | | | | | | |
| 6 | 2,4- | 1 | EtOAc | NP | NP | Coformer | NP |
| | Dihydroxybenzoic | | | | | | |
| | acid | | | | | | |
| 7 | 2,4- | 2 | EtOAc | NP | NP | Coformer | NP |
| | Dihydroxybenzoic | | | | | | |
| | acid | | | | | | |
| 8 | Gentisic acid | 1.1 | IPA | Solution | NP | NP | NP |
| 9 | Gentisic acid | 2 | IPA | Solution | NP | NP | NP |
| 10 | Gentisic acid | 1.1 | EtOAc | GEN Form A | GEN Form A | NP | NP |
| 11 | Gentisic acid | 2 | EtOAc | Amorphous | NP | NP | NP |
| 12 | Gentisic acid | 1 | EtOAc | NP | NP | GEN Form A | GEN Form A |
| 13 | Acetic acid | 1.1 | IPA | Solution | NP | NP | NP |
| 14 | Acetic acid | 2 | IPA | Solution | NP | NP | NP |
| 15 | Acetic acid | 1.1 | EtOAc | Form 2 | Form 2 | NP | NP |
| 16 | Acetic acid | 2 | EtOAc | Form 2 | Form 2 + | NP | NP |
| | | | | | Form 3 | | |
| | | | | | (minor) | | |
| 17 | Acetic acid | 1 | EtOAc | NP | NP | Form 3 | Form 3 |
| 18 | Benzenesulfonic | 1.1 | IPA | Solution | NP | NP | NP |
| | acid | | | | | | |
| 19 | Benzenesulfonic | 2 | IPA | Solution | NP | NP | NP |
| | acid | | | | | | |
| 20 | Benzenesulfonic | 1.1 | EtOAc | Solution | NP | NP | NP |
| | acid | | | | | | |
| 21 | Benzenesulfonic | 2 | EtOAc | Amorphous | NP | NP | NP |
| | acid | | | | | | |
| 22 | Benzenesulfonic | 1 | EtOAc | NP | NP | Oil | NP |
| | acid | | | | | | |
| 23 | Hippuric acid | 1.1 | IPA | Solution | NP | NP | NP |
| 24 | Hippuric acid | 2 | IPA | Solution | NP | NP | NP |
| 25 | Hippuric acid | 1.1 | EtOAc | Solution | NP | NP | NP |
| 26 | Hippuric acid | 2 | EtOAc | Coformer | NP | NP | NP |
| 27 | Hippuric acid | 1 | EtOAc | NP | NP | HIP Form A | HIP Form A |
| 28 | Maleic acid | 1.1 | IPA | Solution | NP | NP | NP |
| 29 | Maleic acid | 2 | IPA | Solution | NP | NP | NP |
| 30 | Maleic acid | 1.1 | EtOAc | Solution | NP | NP | NP |
| 31 | Maleic acid | 2 | EtOAc | Solution | NP | NP | NP |
| 32 | Maleic acid | 0.5 | EtOAc | NP | NP | Oil | NP |
| 33 | Maleic acid | 1 | EtOAc | NP | NP | Oil | NP |
| 34 | Nicotinic acid | 1.1 | IPA | Solution | NP | NP | NP |
| 35 | Nicotinic acid | 2 | IPA | Coformer | NP | NP | NP |
| 36 | Nicotinic acid | 1.1 | EtOAc | Solution | NP | NP | NP |
| 37 | Nicotinic acid | 2 | EtOAc | Coformer | NP | NP | NP |
| 38 | Nicotinic acid | 1 | EtOAc | NP | NP | Oil | NP |
| 39 | Orotic acid | 1.1 | IPA | ORO Form A + | ORO Form B + | NP | NP |
| | | | | ORO Form B | ORO Form A | | |
| | | | | | (traces) | | |
| 40 | Orotic acid | 1.1 | IPA | ORO Form C | ORO Form C | NP | NP |
| 41 | Orotic acid | 2 | IPA | ORO Form B + | ORO Form B + | NP | NP |
| | | | | coformer | coformer | | |
| | | | | (minor) | (minor) | | |
| 42 | Orotic acid | 1.1 | EtOAc | ORO Form A + | ORO Form A + | NP | NP |
| | | | | ORO Form B | ORO Form B | | |
| 43 | Orotic acid | 1.1 | EtOAc | ORO Form A | ORO Form A | NP | NP |
| 44 | Orotic acid | 2 | EtOAc | ORO Form A | ORO Form A | NP | NP |
| | | | | | + coformer | | |
| 45 | Orotic acid | 1 | EtOAc | NP | NP | ORO Form A | ORO Form A |
| 46 | Succinic acid | 1.1 | IPA | Solution | NP | NP | NP |
| 47 | Succinic acid | 2 | IPA | Solution | NP | NP | NP |
| 48 | Succinic acid | 1.1 | EtOAc | Solution | NP | NP | NP |
| 49 | Succinic acid | 2 | EtOAc | Coformer | NP | NP | NP |
| 50 | Succinic acid | 0.5 | EtOAc | NP | NP | Oil | NP |
| 51 | Succinic acid | 1 | EtOAc | NP | NP | Oil | NP |
| 52 | 2,4,6- | 1.1 | IPA | Solution | NP | NP | NP |
| | Trimethylphenol | | | | | | |
| 53 | 2,4,6- | 1.1 | EtOAc | Solution | NP | NP | NP |
| | Trimethylphenol | | | | | | |
| 54 | 2,4-Lutidine | 1.1 | IPA | Solution | NP | NP | NP |
| 55 | 2,4-Lutidine | 1.1 | EtOAc | Solution | NP | NP | NP |
| 56 | 3-Ethoxy-4- | 1.1 | IPA | Red solution | NP | NP | NP |
| | hydroxybenzaldeh | | | | | | |
| | yde | | | | | | |
| 57 | Nicotinamide | 1.1 | EtOAc | Coformer | NP | NP | NP |
| 58 | m-Cresol | 1.1 | IPA | Solution | NP | NP | NP |
| 59 | m-Cresol | 1.1 | EtOAc | Solution | NP | NP | NP |
| 60 | p-Cresol | 1.1 | IPA | Solution | NP | NP | NP |
| 61 | p-Cresol | 1.1 | EtOAc | Solution | NP | NP | NP |
| 62 | o-Sulfobenzimide | 1.1 | IPA | Solution | NP | NP | NP |
| 63 | o-Sulfobenzimide | 1.1 | EtOAc | Solution | NP | NP | NP |
| 64 | Urea | 1.1 | IPA | Solution | NP | NP | NP |
| 65 | Urea | 1.1 | EtOAc | Solution | NP | NP | NP |
| 66 | 4-Aminobenzoic | 1.1 | IPA | Solution | NP | NP | NP |
| | acid | | | | | | |
| 67 | 4-Aminobenzoic | 2 | IPA | Solution | NP | NP | NP |
| | acid | | | | | | |
| 68 | 4-Aminobenzoic | 1.1 | EtOAc | Form 2 | Form 2 + | NP | NP |
| | acid | | | | coformer | | |
| 69 | 4-Aminobenzoic | 2 | EtOAc | Form 2 | Form 2 + | NP | NP |
| | acid | | | | coformer | | |
| 70 | 4-Aminobenzoic | 1 | EtOAc | NP | NP | Oil | NP |
| | acid | | | | | | |
| 71 | Ketoglutaric acid | 1.1 | IPA | Solution | NP | NP | NP |
| 72 | Ketoglutaric acid | 2 | IPA | Solution | NP | NP | NP |
| 73 | Ketoglutaric acid | 1.1 | EtOAc | Solution | NP | NP | NP |
| 74 | Ketoglutaric acid | 2 | EtOAc | Amorphous | NP | NP | NP |
| 75 | Ketoglutaric acid | 0.5 | EtOAc | NP | NP | Oil | NP |
| 76 | Ketoglutaric acid | 1 | EtOAc | NP | NP | Oil | NP |
| 77 | D-Glucuronic acid | 1.1 | IPA | Solution | NP | NP | NP |
| 78 | D-Glucuronic acid | 1.1 | EtOAc | Solution | NP | NP | NP |
| 79 | Vanillin | 1.1 | IPA | Solution | NP | NP | NP |
| 80 | Vanillin | 1.1 | EtOAc | Solution | NP | NP | NP |
| 81 | 2,6-Lutidine | 1.1 | IPA | Solution | NP | NP | NP |
| 82 | 2,6-Lutidine | 1.1 | EtOAc | Solution | NP | NP | NP |
| 83 | Betaine | 1.1 | IPA | Solution | NP | NP | NP |
| 84 | Betaine | 1.1 | EtOAc | Solution | NP | NP | NP |
| 85 | Trimethylamine | 1.1 | IPA | Solution | NP | NP | NP |
| | N-Oxide | | | | | | |
| 86 | Trimethylamine | 1.1 | EtOAc | Solution | NP | NP | NP |
| | N-Oxide | | | | | | |
| 87 | Citric acid | 2 | IPA | Coformer | NP | NP | NP |
| 88 | Citric acid | 2 | EtOAc | Coformer | NP | NP | NP |
| 89 | Citric acid | 0.5 | EtOAc | NP | NP | Oil | NP |
| 90 | Citric acid | 1 | EtOAc | NP | NP | Coformer | NP |
| 91 | Citric acid | 1 | EtOAc | NP | NP | Oil | NP |
| 92 | Citric acid | 2 | EtOAc | NP | NP | Coformer | NP |
| 93 | L-Tartaric acid | 2 | IPA | Coformer | NP | NP | NP |
| 94 | L-Tartaric acid | 2 | EtOAc | TAR Form A | TAR Form A + | NP | NP |
| | | | | | coformer | | |
| 95 | L-Tartaric acid | 0.5 | EtOAc | NP | NP | TAR Form A | TAR Form A |
| 96 | L-Tartaric acid | 1 | EtOAc | NP | NP | TAR Form A + | NP |
| | | | | | | TAR Form B + | |
| | | | | | | Form 1 + | |
| | | | | | | coformer | |
| 97 | L-Tartaric acid | 1 | EtOAc | NP | NP | TAR Form A | TAR Form A |
| 98 | L-Tartaric acid | 2 | EtOAc | NP | NP | TAR Form B + | TAR Form B + |
| | | | | | | TAR Form A | TAR Form A |
| | | | | | | (minor) | (minor) + |
| | | | | | | | coformer |
| 99 | L-Tartaric acid | 2 | EtOAc | NP | NP | TAR Form A | TAR Form A |
| | | | | | | + coformer | |
| 100 | DL-Tartaric acid | 0.5 | EtOAc | NP | NP | Coformer | NP |
| 101 | DL-Tartaric acid | 1 | EtOAc | NP | NP | Coformer | NP |
| 102 | D-Tartaric acid | 0.5 | EtOAc | NP | NP | Oil | NP |
| 103 | D-Tartaric acid | 2 | EtOAc | NP | NP | Oil | NP |
| 104 | L-Aspartic acid | 2 | IPA | Coformer | NP | NP | NP |
| 105 | L-Aspartic acid | 2 | EtOAc | Coformer | NP | NP | NP |
| 106 | L-Aspartic acid | 0.5 | EtOAc | NP | NP | Amorphous | NP |
| 107 | L-Aspartic acid | 1 | EtOAc | NP | NP | Form 1 + | NP |
| | | | | | | coformer | |
| 108 | Benzoic acid | 2 | IPA | Solution | NP | NP | NP |
| 109 | Benzoic acid | 2 | EtOAc | Form 2 | NP | NP | NP |
| 110 | Benzoic acid | 1 | EtOAc | NP | NP | Oil | NP |
| 111 | Benzoic acid | 1 | EtOAc | NP | NP | Oil | NP |
| 112 | Benzoic acid | 2 | EtOAc | NP | NP | Oil | NP |
| 113 | Fumaric acid | 1 | EtOAc | FUM Form A + | Form 4 | NP | NP |
| | | | | Form 4 | | | |
| 114 | Fumaric acid | 0.5 | EtOAc | NP | NP | Form 4 | Form 4 |
| 115 | Fumaric acid | 1 | EtOAc | NP | NP | Form 4 | Deliquescent |
| 116 | Fumaric acid | 2 | EtOAc | NP | NP | FUM Form A | FUM Form A |
| 117 | Fumaric acid | 2 | EtOAc | NP | NP | Form 4 + | Form 4 + |
| | | | | | | coformer | coformer** |
| 118 | L-Malic acid | 1 | EtOAc | NP | NP | Coformer | NP |
| 119 | L-Malic acid | 1 | EtOAc | NP | NP | Oil | NP |
| 120 | L-Malic acid | 2 | EtOAc | NP | NP | Coformer | NP |
| 121 | L-Malic acid | 2 | EtOAc | NP | NP | Coformer | NP |
| 122 | L-Malic acid | 1 | EtOAc | NP | NP | Oil | NP |
| 123 | Propionic acid | 1 | EtOAc | Form 2 | Form 2 | NP | NP |
| 124 | Propionic acid | 1 | EtOAc | NP | NP | PRO Form A | PRO Form A** |
| 125 | Propionic acid | 2 | EtOAc | NP | NP | Oil | NP |
| 126 | Sorbic acid | 1 | EtOAc | NP | NP | Oil | NP |
| 127 | Sorbic acid | 2 | EtOAc | NP | NP | Oil | NP |
| 128 | L-Ascorbic acid | 1 | EtOAc | NP | NP | Oil | NP |
| 129 | L-Ascorbic acid | 2 | EtOAc | NP | NP | Coformer | NP |
| 130 | L-Lactic acid | 1 | EtOAc | NP | NP | Oil | NP |
| 131 | L-Lactic acid | 2 | EtOAc | NP | NP | Oil | NP |
| 132 | Octanoic acid | 1 | EtOAc | NP | NP | Oil | NP |
| 133 | Octanoic acid | 2 | EtOAc | NP | NP | Oil | NP |
| 134 | L-Glutamic acid | 0.5 | EtOAc | NP | NP | Oil | NP |
| 135 | L-Glutamic acid | 1 | EtOAc | NP | NP | Oil | NP |
| 136 | L-Glutamic acid | 2 | EtOAc | NP | NP | Coformer | NP |
| 137 | Glutaric Acid | 0.5 | EtOAc | NP | NP | Oil | NP |
| 138 | Glutaric Acid | 1 | EtOAc | NP | NP | Oil | NP |
| 139 | Glutaric Acid | 2 | EtOAc | NP | NP | Oil | NP |
| 140 | p-Toluenesulfonic | 1 | EtOAc | NP | NP | Oil | NP |
| | acid monohydrate | | | | | | |
| 141 | p-Toluenesulfonic | 2 | EtOAc | NP | NP | Oil | NP |
| | acid monohydrate | | | | | | |
| 142 | Methanesulfonic | 1 | EtOAc | NP | NP | Oil | NP |
| | acid | | | | | | |
| 143 | Methanesulfonic | 2 | EtOAc | NP | NP | Oil | NP |
| | acid | | | | | | |
| 144 | 2,3,6- | 1 | EtOAc | NP | NP | Oil | NP |
| | Trimethylphenol | | | | | | |
| 145 | 2,3,6- | 2 | EtOAc | NP | NP | Oil | NP |
| | Trimethylphenol | | | | | | |
| 146 | 4-ally1-2,6- | 1 | EtOAc | NP | NP | Oil | NP |
| | dimethoxyphenol | | | | | | |
| 147 | 4-ally1-2,6- | 2 | EtOAc | NP | NP | Oil | NP |
| | dimethoxyphenol | | | | | | |
| 148 | 4-tert-Butylphenol | 1 | EtOAc | NP | NP | Oil | NP |
| 149 | 4-tert-Butylphenol | 2 | EtOAc | NP | NP | Oil | NP |
| 150 | Hydrochloric acid | 1 | EtOAc | NP | NP | Oil | NP |
| 151 | Hydrochloric acid | 2 | EtOAc | NP | NP | Oil | NP |
| 152 | Galactaric acid | 1 | EtOAc | NP | NP | Coformer | NP |
| 153 | Galactaric acid | 2 | EtOAc | NP | NP | Coformer | NP |
| 154 | Phosphoric acid | 1 | EtOAc | NP | NP | Coformer | NP |
| 155 | Phosphoric acid | 2 | EtOAc | NP | NP | Coformer | NP |
| 156 | Adipic acid | 0.5 | EtOAc | NP | NP | ADI Form A | ADI Form A |
| 157 | Adipic acid | 0.5 | EtOAc | ADI Form A | NP | NP | NP |
| 158 | Adipic acid | 1 | EtOAc | NP | NP | ADI Form A | ADI Form |
| | | | | | | | A** |
| 159 | Adipic acid | 1 | EtOAc | Solution | Solution | NP | NP |
| 160 | Adipic acid | 2 | EtOAc | NP | NP | ADI Form B + | Coformer |
| | | | | | | coformer | |
| 161 | Linoleic acid | 1 | EtOAc | NP | NP | Oil | NP |
| 162 | Linoleic acid | 2 | EtOAc | NP | NP | Oil | NP |
| 163 | DL-Malic acid | 0.5 | EtOAc | NP | NP | Form 4 | Form 4 |
| 164 | DL-Malic acid | 1 | EtOAc | NP | NP | Form 4 | Deliquescent |
| 165 | DL-Malic acid | 1 | EtOAc | Solution | Solution | NP | NP |
| 166 | DL-Malic acid | 2 | EtOAc | NP | NP | Form 4 + | Form 4 + |
| | | | | | | coformer | coformer** |
| 167 | Mucic acid | 0.5 | EtOAc | NP | NP | MUC Form A + | MUC Form A |
| | | | | | | MUC Form B + | |
| | | | | | | Form 1 | |
| 168 | Mucic acid | 0.5 | EtOAc | MUC Form B | MUC Form B | NP | NP |
| 169 | 2-Methoxybenzoic | 1 | EtOAc | NP | NP | 2-MXB Form | 2-MXB Form |
| | acid | | | | | A | A |
| 170 | 2-Methoxybenzoic | 1 | EtOAc | Form 2 | NP | NP | NP |
| | acid | | | | | | |
| 171 | 2-Methoxybenzoic | 2 | EtOAc | NP | NP | 2-MXB Form | 2-MXB Form |
| | acid | | | | | A | A |
| 172 | 2-Methoxybenzoic | 2 | EtOAc | Form 2 | NP | NP | NP |
| | acid | | | | | | |
| 173 | 3-Methoxybenzoic | 1 | EtOAc | NP | NP | 3-MXB Form | 3-MXB Form |
| | acid | | | | | A | A |
| 174 | 3-Methoxybenzoic | 1 | EtOAc | Form 2 | NP | NP | NP |
| | acid | | | | | | |
| 175 | 3-Methoxybenzoic | 2 | EtOAc | NP | NP | 3-MXB Form | 3-MXB Form |
| | acid | | | | | A | A |
| 176 | 3-Methoxybenzoic | 2 | EtOAc | Form 2 | NP | NP | NP |
| | acid | | | | | | |
| 177 | 4-Methoxybenzoic | 1 | EtOAc | NP | NP | Coformer | NP |
| | Acid | | | | | | |
| 178 | 4-Methoxybenzoic | 2 | EtOAc | NP | NP | Coformer | NP |
| | Acid | | | | | | |
| 179 | 3,4- | 1 | EtOAc | NP | NP | Coformer | NP |
| | Dihydroxybenzoic | | | | | | |
| | Acid | | | | | | |
| 180 | 3,4- | 2 | EtOAc | NP | NP | Coformer | NP |
| | Dihydroxybenzoic | | | | | | |
| | Acid | | | | | | |
| 181 | 3-Hydroxybenzoic | 1 | EtOAc | NP | NP | 3-HXB Form | 3-HXB Form |
| | acid | | | | | A | A |
| 182 | 3-Hydroxybenzoic | 1 | EtOAc | 3-HXB Form | 3-HXB Form | NP | NP |
| | acid | | | A | A | | |
| 183 | 3-Hydroxybenzoic | 2 | EtOAc | NP | NP | 3-HXB Form | 3-HXB Form |
| | acid | | | | | A | A |
| 184 | 3-Hydroxybenzoic | 2 | EtOAc | 3-HXB Form | 3-HXB Form | NP | NP |
| | acid | | | A | A | | |

*Experiments performed with the compound of Formula (I) Form 1.
**Solids not stable at ambient conditions (25 ± 1° C./70 ± 5% RH). Deliquescence was observed.

Results from the salt/cocrystal screening study are summarized in Table 28 below.

TABLE 28
Summary of Salt/Cocrystal Screen Experiments

| | | Acid | | | | | |
|---|---|---|---|---|---|---|---|
| Refer- | | equi- | | Heat-Cool Cycling | | Wet Grinding* | |
| ence | Coformer | valents | Solvent | Wet | Dry | Wet | Dry |
| 1 | Orotic acid | 1.1 | IPA | ORO Form A + | ORO Form B + | NP | NP |
| | | | | ORO Form B | ORO Form A | | |
| | | | | | (traces) | | |
| 2 | Orotic acid | 1.1 | IPA | ORO Form C | ORO Form C | NP | NP |
| 3 | Orotic acid | 2.0 | IPA | ORO Form B + | ORO Form B + | NP | NP |
| | | | | coformer | coformer | | |
| | | | | (minor) | (minor) | | |
| 4 | Orotic acid | 1.1 | EtOAc | ORO Form A + | ORO Form A + | NP | NP |
| | | | | ORO Form B | ORO Form B | | |
| 5 | Orotic acid | 2.0 | EtOAc | ORO Form A | ORO Form A + | NP | NP |
| | | | | | coformer | | |
| 6 | Orotic acid | 1.1 | EtOAc | ORO Form A | ORO Form A | NP | NP |
| 7 | Gentisic | 1.0 | EtOAc | NP | NP | GEN Form A | GEN Form A |
| | acid | | | | | | |
| 8 | Hippuric | 1.0 | EtOAc | NP | NP | HIP Form A | HIP Form A |
| | acid | | | | | | |
| 9 | L-Tartaric | 2.0 | EtOAc | TAR Form A | TAR Form A + | NP | NP |
| | acid | | | | coformer | | |
| 10 | L-Tartaric | 1.0 | EtOAc | NP | NP | TAR Form A + | NP |
| | acid | | | | | TAR Form B + | |
| | | | | | | Form 1 + | |
| | | | | | | coformer | |
| 11 | L-Tartaric | 2.0 | EtOAc | NP | NP | TAR Form B + | TAR Form B + |
| | acid | | | | | TAR Form A | TAR Form A |
| | | | | | | (minor) | (minor) + |
| | | | | | | | coformer |
| 12 | L-Tartaric | 1.0 | EtOAc | NP | NP | TAR Form A | TAR Form A |
| | acid | | | | | | |
| 13 | L-Tartaric | 2.0 | EtOAc | NP | NP | TAR Form A + | TAR Form A |
| | acid | | | | | coformer | |
| 14 | L-Tartaric | 0.5 | EtOAc | NP | NP | TAR Form A | TAR Form A |
| | acid | | | | | | |
| 15 | Fumaric | 2.0 | EtOAc | NP | NP | FUM Form A | FUM Form A |
| | acid | | | | | | |
| 16 | Fumaric | 1.0 | EtOAc | NP | NP | Form 4 | Deliquescent |
| | acid | | | | | | |
| 17 | Fumaric | 2.0 | EtOAc | NP | NP | Form 4 + | Form 4 + |
| | acid | | | | | coformer | coformer** |
| 18 | Fumaric | 0.5 | EtOAc | NP | NP | Form 4 | Form 4 |
| | acid | | | | | | |
| 19 | DL-Malic | 1.0 | EtOAc | NP | NP | Form 4 | Deliquescent |
| | acid | | | | | | |
| 20 | DL-Malic | 2.0 | EtOAc | NP | NP | Form 4 + | Form 4 + |
| | acid | | | | | coformer | coformer** |
| 21 | DL-Malic | 0.5 | EtOAc | NP | NP | Form 4 | Form 4 |
| | acid | | | | | | |
| 22 | Mucic acid | 0.5 | EtOAc | NP | NP | MUC Form A + | MUC Form A |
| | | | | | | MUC Form B + | |
| | | | | | | Form 1 | |
| 23 | Mucic acid | 0.5 | EtOAc | MUC Form B | MUC Form B | NP | NP |
| 24 | Adipic acid | 1.0 | EtOAc | NP | NP | ADI Form A | ADI Form A** |
| 25 | Adipic acid | 2.0 | EtOAc | NP | NP | ADI Form B + | Coformer |
| | | | | | | coformer | |
| 26 | Adipic acid | 0.5 | EtOAc | NP | NP | ADI Form A | ADI Form A |
| 27 | Adipic acid | 0.5 | EtOAc | ADI Form A | NP | NP | NP |
| 28 | Propionic | 1.0 | EtOAc | NP | NP | PRO Form A | PRO Form A** |
| | acid | | | | | | |
| 29 | 2- | 1.0 | EtOAc | NP | NP | 2-MXB Form | 2-MXB Form |
| | Methoxyben | | | | | A | A |
| | zoic acid | | | | | | |
| 30 | 2- | 1.0 | EtOAc | Form 2 | NP | NP | NP |
| | Methoxyben | | | | | | |
| | zoic acid | | | | | | |
| 31 | 2- | 2.0 | EtOAc | NP | NP | 2-MXB Form | 2-MXB Form |
| | Methoxyben | | | | | A | A |
| | zoic acid | | | | | | |
| 32 | 2- | 2.0 | EtOAc | Form 2 | NP | NP | NP |
| | Methoxyben | | | | | | |
| | zoic acid | | | | | | |
| 33 | 3- | 1.0 | EtOAc | NP | NP | 3-MXB Form | 3-MXB Form |
| | Methoxyben | | | | | A | A |
| | zoic acid | | | | | | |
| 34 | 3. | 1.0 | EtOAc | Form 2 | NP | NP | NP |
| | Methoxyben | | | | | | |
| | zoic acid | | | | | | |
| 35 | 3- | 2.0 | EtOAc | NP | NP | 3-MXB Form | 3-MXB Form |
| | Methoxyben | | | | | A | A |
| | zoic acid | | | | | | |
| 36 | 3- | 2.0 | EtOAc | Form 2 | NP | NP | NP |
| | Methoxyben | | | | | | |
| | zoic acid | | | | | | |
| 37 | 3- | 1.0 | EtOAc | NP | NP | 3-HXB Form | 3-HXB Form |
| | Hydroxyben | | | | | A | A |
| | zoic acid | | | | | | |
| 38 | 3- | 1.0 | EtOAc | 3-HXB Form | 3-HXB Form | NP | NP |
| | Hydroxyben | | | A | A | | |
| | zoic acid | | | | | | |
| 39 | 3- | 2.0 | EtOAc | NP | NP | 3-HXB Form | 3-HXB Form |
| | Hydroxyben | | | | | A | A |
| | zoic acid | | | | | | |
| 40 | 3- | 2.0 | EtOAc | 3-HXB Form | 3-HXB Form | NP | NP |
| | Hydroxyben | | | A | A | | |
| | zoic acid | | | | | | |
| 41 | Acetic acid | 1.1 | EtOAc | Form 2 | Form 2 | NP | NP |
| 42 | Acetic acid | 2.0 | EtOAc | Form 2 | Form 2 + | NP | NP |
| | | | | | Form 3 | | |
| | | | | | (minor) | | |
| 43 | Acetic acid | 1.0 | EtOAc | NP | NP | Form 3 | Form 3 |
| 44 | 4- | 1.1 | EtOAc | Form 2 | Form 2 + | NP | NP |
| | Aminobenz | | | | Coformer | | |
| | oic acid | | | | | | |
| 45 | 4- | 2.0 | EtOAc | Form 2 | Form 2 + | NP | NP |
| | Aminobenz | | | | Coformer | | |
| | oic acid | | | | | | |
| 46 | Benzoic | 2.0 | EtOAc | Form 2 | NP | NP | NP |
| | acid | | | | | | |

*Experiments performed with the compound of Formula (I) Form 1.
**Solids not stable at ambient conditions (25 ± 1° C./70 ± 5% RH). Deliquescence was observed.

From the salt screening experiments, 9 different crystalline forms were observed in 8 different acids. It is worth noting that with orotic acid three different crystalline forms were observed and with L-tartaric acid two different crystalline forms were observed. With acetic acid, 4-aminobenzoic acid and benzoic acid the same crystalline form, the compound of Formula (I) Form 2, was observed, which was suspected to be a polymorph of the compound of Formula (I). Another compound of Formula (I) crystalline form with acetic acid was observed (the compound of Formula (I) Form 3). Crystalline forms MUC Form A, 2-MXB Form A, 3-MXB Form A and 3-HXB Form A were stable under ambient conditions. However, the use of the same coformers by heat-cool cycling (25° C. to 50° C.) lead to the obtention of the compound of Formula (I) Form 2 (polymorph of the compound of Formula (I)) instead of the formation of the corresponding salt, except for the 3-hydroxybenzoic acid, where 3-HXB Form A was obtained.

A new compound of Formula (I) crystalline form, called Form 4, was observed with fumaric acid and DL-Malic, which was suspected to be a polymorph of the compound of Formula (I). This form was not stable at ambient conditions and deliquescence was observed.

Stability tests in the XRPD sample holders at 40° C./75% RH were performed with the crystalline forms that were stable after drying the solid under vacuum at RT overnight (see Table 27) and they were compared with the sulfate salt of the compound of Formula (I). The results are shown in Table 29.

TABLE 29
Stability Study at 40° C./75% RH of Salts in XRPD Sample Holder

| Refer- | | XRPD | XRPD | XRPD | XRPD | XRPD | XRPD |
|---|---|---|---|---|---|---|---|
| ence | Acid | t0 | 1d | 2d | 3d | 6d | 7d |
| 1 | Sulfuric acid | Form A | Deliquescent | NP | NP | NP | NP |
| 2 | Orotic acid | ORO | ORO | ORO | ORO | ORO | ORO |
| | | Form C | Form A + | Form A + | Form A + | Form A + | Form A + |
| | | | ORO | ORO | ORO | ORO | ORO |
| | | | Form D | Form D | Form D | Form D | Form D |
| | | | (minor) | (minor) | (minor) | (minor) | (minor) |
| 3 | Orotic acid | ORO | ORO | ORO | ORO | ORO | ORO |
| | | Form B | Form A + | Form A + | Form A + | Form A + | Form A + |
| | | | ORO | ORO | ORO | ORO | ORO |
| | | | Form D | Form D | Form D | Form D | Form D |
| | | | (minor) | (minor) | (minor) | (minor) | (minor) |
| 4 | Orotic acid | ORO | ORO | ORO | ORO | ORO | ORC |
| | | Form A | Form A + | Form A + | Form A + | Form A + | Form A + |
| | | | ORO | ORO | ORO | ORO | ORO |
| | | | Form D | Form D | Form D | Form D | Form D |
| | | | (minor) | (minor) | (minor) | (minor) | (minor) |
| 5 | L-Tartaric | TAR | TAR | TAR | TAR | TAR | TAR |
| | acid | Form A | Form A | Form A | Form A | Form A | Form A |
| 6 | Gentisic acid | GEN | GEN | GEN | GEN | GEN | GEN |
| | | Form A | Form A | Form A | Form A | Form A | Form A |
| 7 | Hippuric acid | HIP | Deliquescent | NP | NP | NP | NP |
| | | Form A | | | | | |
| 8 | Fumaric acid | FUM | Deliquescent | NP | NP | NP | NP |
| | | Form A | | | | | |
| 9 | Acetic acid | Form 2 | Form 2 | Form 2 | Form 2 | Form 2 | Form 2 |
| 10 | Acetic | Form 2 + | Form 2 | NP | NP | NP | NP |
| | | Form 3 | | | | | |
| | | (minor) | | | | | |
| 11 | 4-Amino- | Form 2 + | Form 2 + | Form 2 + | Form 2 + | Form 2 + | Form 2 + |
| | benzoic | Coformer | Coformer | Coformer | Coformer | Coformer | Coformer |
| | acid | | | | | | |
| 12 | Acetic acid | Form 3 | Deliquescent | NP | NP | NP | NP |
| 13 | Mucic acid | MUC | MUC | MUC | MUC | NP | MUC |
| | | Form A | Form A | Form C | Form C | | Form C |
| 14 | 2-Methoxy- | 2-MXB | Deliquescent | NP | NP | NP | NP |
| | benzoic | Form A | | | | | |
| | Acid | | | | | | |
| 15 | 3-Methoxy- | 3-MXB | Deliquescent | NP | NP | NP | NP |
| | benzoic | Form A | | | | | |
| | Acid | | | | | | |
| 16 | 3-Hydroxy- | 3-HXB | 3-HXB | 3-HXB | NP | 3-HXB | 3-HXB |
| | benzoic | Form A | Form A | Form A | | Form A | Form A |
| | Acid | | | | | | |
| 17 | Adipic acid | ADI | ADI | ADI | NP | NP | ADI |
| | | Form A | Form A | Form A | | | Form A |

Four of the crystalline salts (TAR Form A, GEN Form A, 3-HXB Form A and ADI Form A) stored under accelerated conditions at 40° C./75% RH were stable for at least 1 week.

The compound of Formula (I) Form 2, which was suspected to be a polymorph of the compound of Formula (I), was stable at these conditions for 1 week.

ORO Form A, ORO Form B and ORO Form C were not stable after 1 day and a new crystalline form, called ORO Form D, was observed mixed with ORO Form A in the three cases. These mixtures ORO Form A and ORO Form D remained stable after 1 week.

MUC Form A transformed after 2 days into a new crystalline form, called MUC Form C, which was stable for at least 1 week at 40° C./75% RH.

Five of the crystalline forms (hippurate salt, fumarate salt, 2-methoxybenzoate salt, 3-methoxybenzoate salt and Form 3) were deliquescent after 1 day of storage at these conditions. Similarly, the sulfate salt showed deliquescence after 1 day.

Additionally, the salts that were stable at 40° C./75% RH were stored at 25° C./96% RH in an XRPD sample holder. The results are shown in Table 30.

TABLE 30
Stability Study at 25° C./96% RH of Salts in XRPD Sample Holders

| | | XRPD | | | XRPD | XRPD | |
|---|---|---|---|---|---|---|---|
| Reference | Acid | t0 | XRPD 1d | XRPD 2d | 3d | 6d | XRPD 7d |
| 1 | Orotic acid | ORO | ORO Form | ORO Form | ORO | ORO | ORO Form |
| | | Form A | A + ORO | A + ORO | Form | Form | A + ORO |
| | | | Form D | Form D | A + | A + | Form D |
| | | | | | ORO | ORO | |
| | | | | | Form | Form | |
| 2 | L-Tartaric acid | TAR | Deliquescent | NP | NP | NP | NP |
| | | Form A | | | | | |
| 3 | Gentisic acid | GEN | GEN Form | GEN Form | GEN | GEN | GEN Form |
| | | Form A | A | A | Form | Form | A |
| | | | | | A | A | |
| 4 | 3- | 3-HXB | 3-HXB | 3-HXB Form | NP | NP | Deliquescent |
| | Hydroxybenzoic | Form A | Form A | A | | | |
| | acid | | | | | | |
| 5 | Adipic acid | ADI | ADI Form A | Deliquescent | NP | NP | NP |
| | | Form A | | | | | |

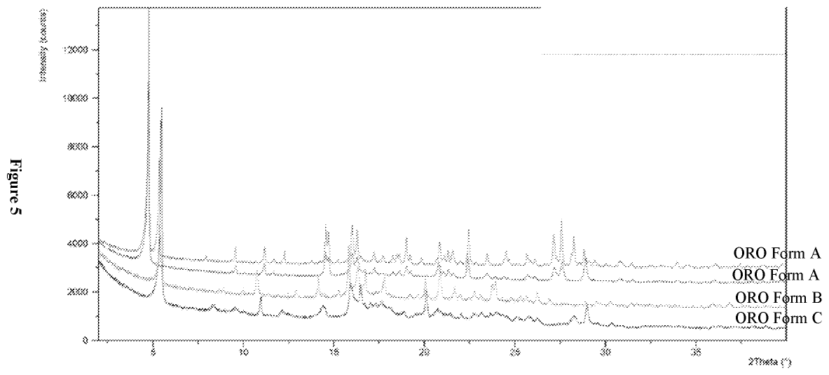

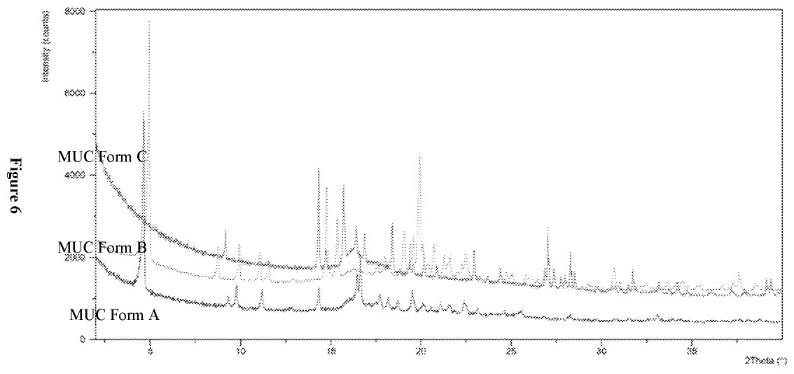

ORO Form A was transformed partially into ORO Form D after 1 day of storage at 25° C./96% RH conditions (see FIG. 5).

Only the crystalline salt GEN Form A stored under accelerated conditions at 25° C./96% RH was stable for at least 1 week.

The L-tartrate and adipate salts were deliquescent after 1 or 2 days of storage at 25° C./96% RH conditions.

3-HXB Form A was stable for at least 2 days at 25° C./96% RH, but deliquescence was observed after 7 days of storage.

### Further Experiments Relating to Form 2 and Form 3

As described above, a new crystalline form, called Form 2, was obtained with 3 different coformers, and therefore it was suspected to be a polymorph of the compound of Formula (I), instead of a salt/cocrystal. These experiments were repeated with the addition of a blank (without conformer). The results are summarized in Table 31. Form 2 was seen with 4-aminobenzoic acid and benzoic acid, however this time in acetic acid a different form was seen, Form 3. It is assigned as a polymorph of the compound of Formula (I).

An attempt was made to reproduce Form 2 at 1 g scale, but a solution was observed in this case.

TABLE 31
Additional Experiments to Obtain Forms 2 and 3

| | | | Scale | | Vol | XRPD | |
|---|---|---|---|---|---|---|---|
| Reference | Acid | Equivalents | (mg) | Solvent | (V) | wet | XRPD dry |
| 1 | Acetic acid | 1 | 50 | EtOAc | 5 | Form 3 | Form 3 |
| 2 | 4-Aminobenzoic | 1 | 50 | EtOAc | 3 | Form 2 | Deliquescent |
| | acid | | | | | | |
| 3 | Benzoic acid | 1 | 50 | EtOAc | 3 | Form 2 | Deliquescent |
| 4 | — | — | 100 | EtOAc | 2 | Form 2 | Form 2* |
| 5 | — | — | 1000 | EtOAc | 1 | Solution | Solution |

*Sticky solid

### Summary of Example 2

Of the coformers initially tested in the salt screen with oily compound of Formula (I) or Form 1, 9 new crystalline forms were obtained with different acids. It was suspected that 2 of these 9 crystalline forms (Form 2 and Form 3) were polymorphs of the compound of Formula (I). Of these 9 new forms, most of them were not stable under accelerated conditions at 40° C./75% RH and deliquescence was observed. The crystalline salt with gentisic acid (GEN Form A) was stable for at least 1 week at 40° C./75% RH while a mixture of forms was observed after 1 day for the salts prepared with orotic acid (ORO Forms A, B and C) and deliquescence was observed after 1 day for the salt with L-tartaric acid (TAR Form A). At 25° C./96% RH, GEN Form A was stable for at least 1 week. The L-tartrate salt (TAR Form A) was deliquescent after 1 day at these conditions while a mixture of ORO Form A and a new crystalline form, called ORO Form D, was observed after 1 day. Crystalline Form 2 was stable for 1 week at 40° C./75% RH while Form 3 was deliquescent after 1 day. Form 2 was reproduced at 100 mg scale (sticky solid obtained) without coformer confirming that it is a polymorph of the compound of Formula (I). However, it was not possible to obtain Form 2 at 1 g scale. From the salt screen, 8 new crystalline forms were observed and 5 of them were stable under ambient conditions (MUC Form A, 2-MXB Form A, 3-MXB Form A, 3-HXB Form A and ADI Form A). It was suspected that 1 of these new crystalline forms (Form 4) was a polymorph of the compound of Formula (I). Form 3-HXB Form A and ADI Form A were stable for at least 1 week at 40° C./75% RH. MUC Form A transformed completely into MUC Form C after 2 days under these conditions. At 25° C./96% RH, 3-HXB Form A and ADI Form A were deliquescent after 2 to 7 days. The crystalline salt with adipic acid (ADI Form A) was surprisingly stable for at least 1 week at high temperature and humidity (40° C./75% RH, while at 25° C./96% RH deliquescence was observed after 2 days).

### Example 3: Scale Up and Additional Characterization

### Preparation at 50-250 mg Scale

ORO Form A, ORO Form B, TAR Form A, GEN Form A, 3-HXB Form A, MUC Form A and ADI Form A were prepared at 50-250 mg scale for further characterization using the compound of Formula (I) Form 1. Heat-cool cycles between 25° C. to 50° C. (30 min at each temperature, with heating and cooling at 1° C./min) for 1 day were performed. The procedures were not optimized. The experiments are described in Table 32.

TABLE 32
Summary of Experiments Performed at 100-250 mg Scale

| | | Equiv | Scale | | | |
|---|---|---|---|---|---|---|
| Reference | Acid | alents | (mg) | Solvent | XRPD wet | XRPD dry |
| 1 | Orotic acid | 1 | 50 | EtOAc | ORO Form A | ORO Form A |
| 2 | Orotic acid | 1 | 250 | EtOAc | ORO Form A | ORO Form A |
| 3 | Orotic acid | 1 | 50 | IPA | ORO Form B | ORO Form B |
| 4 | Orotic acid | 1 | 250 | IPA | ORO Form B + | ORO Form B + ORO |
| | | | | | ORO Form C | Form C |
| 5 | L-Tartaric | 1 | 50 | EtOAc | TAR Form A + | TAR Form A |
| | acid | | | | TAR Form B | |
| 6 | L-Tartaric | 1 | 100 | EtOAc | TAR Form A + | TAR Form B + TAR |
| | acid | | | | TAR Form B | Form A + Form 1 + |
| | | | | | | coformer |
| 7 | Gentisic acid | 1 | 100 | EtOAc | GEN Form A | GEN Form A |
| 8 | 3- | 1 | 250 | EtOAc | 3-HXB Form | 3-HXB Form A |
| | Hydroxybenzoic | | | | A | |
| | acid | | | | | |
| 9* | Mucic acid | 1 | 100 | EtOAc | MUC form C + | Amorphous |
| | | | | | MUC Form A | |
| 10* | Adipic acid | 1 | 250 | EtOAc | ADI Form A | ADI Form A |

*Sticky solid recovered after the filtration.

ORO Form A, GEN Form A, 3-HXB Form A and ADI Form A were reproduced successfully.

The preparation of ORO Form B afforded a mixture of ORO Form B and ORO Form C. When TAR Form A was reproduced at 100 mg scale, some disproportionation occurred giving a mixture of Form 1 of the compound of Formula (I), tartaric acid, together with TAR Form A and TAR Form B.

The preparation of MUC Form A afforded in wet a mixture of MUC Form C and MUC Form A. However, after drying the solid an amorphous pattern was obtained by XRPD.

### Preparation at 500 mg or 1 g Scale

ORO Form A, GEN Form A, TAR Form A, and ADI Form A were prepared at 1 g scale and 3-HXB Form A and TAR Form A were prepared at 500 mg scale, to complete the characterization and perform a polymorphism assessment and a solubility study in water at different pHs. A stability study at different humidity and temperature conditions was also performed. In these scale up experiments the compound of Formula (I) was added as a solid and the processes used were based on the screening procedures. The experiments are described in Table 33.

TABLE 33
Summary of Experiments Performed at 500 mg or 1 g Scale

| Reference | Acid | Equivalents | Solvent | Vol (V) | XRPD wet | XRPD dry |
|---|---|---|---|---|---|---|
| 1 | Orotic | 1 | EtOAc | 5 | ORO Form A | ORO Form A |
| 2 | Gentisic | 1 | EtOAc | 5 + 15 | GEN Form A | GEN Form A |
| 3 | L-Tartaric acid** | 1 | EtOAc | 5 | TAR Form A | TAR Form A + TAR |
| | | | | | | Form B (minor) |
| 4 | L-Tartaric acid*** | 1 | Acetone | 5 | TAR Form A + | NP |
| | | | | | coformer* | |
| 5 | L-Tartaric acid*** | 0.9 | EtOAc | 5 | TAR Form A | TAR Form A |
| 6 | 3-Hydroxybenzoic | 1 | EtOAc | 20 + 5 | 3-HXB Form | 3-HXB Form A |
| | acid | | | | A | |
| 7 | Adipic acid | 1 | EtOAc | 1 | ADI Form A* | ADI Form A |
| 8 | Adipic acid | 1 | THF | 1 | ADI Form A | ADI Form A |

*Sticky solid obtained.
**1000 mg scale.
***500 mg scale.

### Scale Up of Gentisate Salt

After some initial small-scale trials, the process (starting from the sulfate salt) was scaled up first to 4 g and then to 80-100 g batches in a 1-L jacketed vessel (see Table 34). Approximately 260 g of gentisate salt was prepared from the sulfate in 3 batches, first cracking the sulfate with NaOH and partitioning in DCM-water, concentrating the organic and crystallizing the gentisate in ethanol (4 vol) with seeding at 68° C., followed by cooling to 5° C. and antisolvent addition (EtOAc, 7 vol).

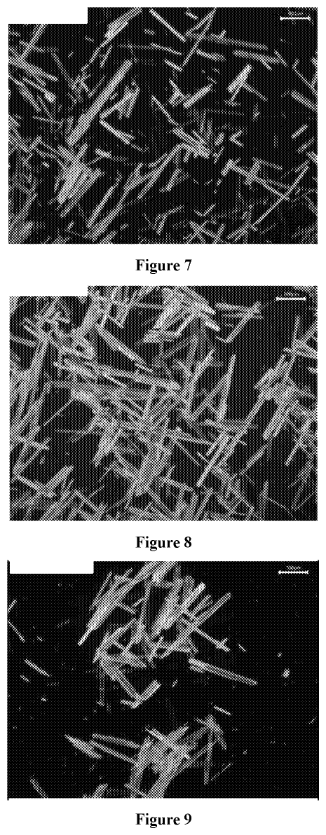

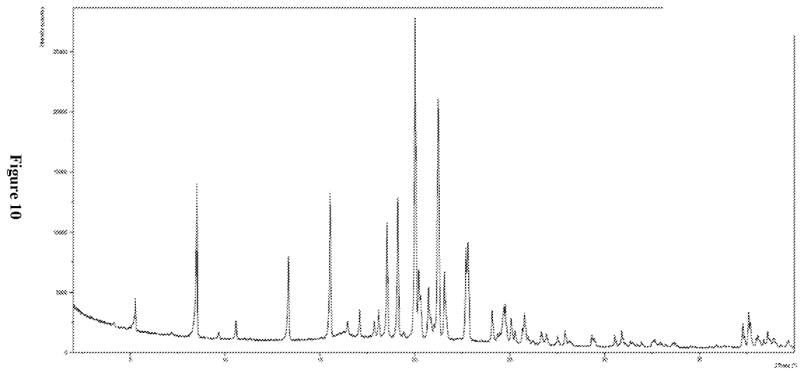

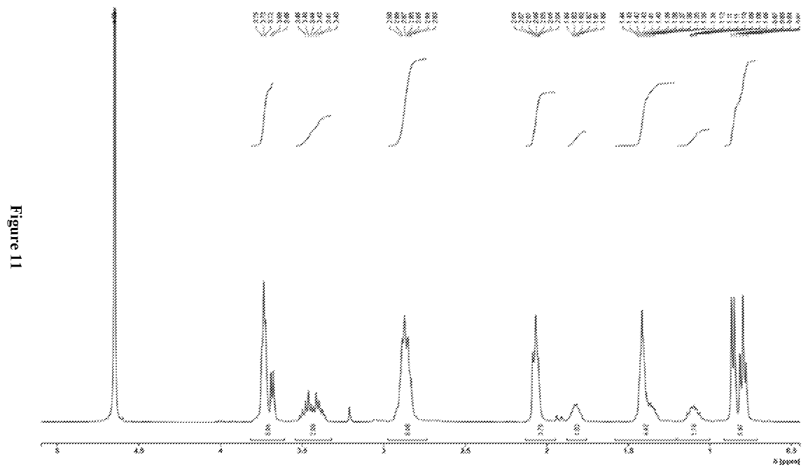

TABLE 34
Scale-up Batches, Gentisate Salt

| Reference | Yield (g, %) | XRPD | 1H NMR | DSC | TGA | Microscopy |
|---|---|---|---|---|---|---|
| 1 | 4 g, 84.1% | GEN Form A | conforms | NP | NP | NP |
| 2 | 78 g, 85.9% | GEN Form A | conforms | Onset 168.2° C. | 0.80% | FIG. 7 |
| | | | | (133 J/g) | | |
| 3 | 82 g, 89.5% | GEN Form A | conforms | Onset 168.6° C. | 0.60% | FIG. 8 |
| | | | | (134 J/g) | | |
| 4 | 99 g, 88.4% | GEN Form A | conforms | Onset 168.6° C. | 0.70% | FIG. 9 |
| | | | | (135 J/g) | | |

### Experimental Procedures—Scale Up

Preparation of ORO Form A. The compound of Formula (I) (1 g) and orotic acid (655.5 mg, 1.0 eq.) were suspended in EtOAc (5 mL, 5 vol). The resulting mixture was subjected to heat-cool cycles between 25° C. and 50° C. with 30 minutes at each temperature with heating and cooling of 1° C./min for 1 day. The mixture was filtered, washed with EtOAc (2×2 mL, 2 vol) and analysed wet by XRPD affording ORO Form A. Then, the solid was dried under vacuum at 25° C. overnight. An off-white solid 897 mg (54.5%) was obtained which corresponded to ORO Form A by XRPD. ORO Form A was also characterized by ¹H NMR, DSC, TGA, DVS and HPLC. A white solid block was observed at the bottom of the vial which was analysed by XRPD affording a mixture of ORO Form A and conformer. The two solids were not mixed.

Preparation of TAR Form A, 500 mg Scale. The compound of Formula (I) (552 mg) and L-tartaric acid (298 mg, 0.9 eq.) were suspended in EtOAc (2.5 mL, 5 vol). The resulting mixture was subjected to heat-cool cycles between 25° C. and 50° C. with 30 minutes at each temperature with heating and cooling of 1° C./min for 1 day. The mixture was filtered, washed with EtOAc (2×1 mL, 2 vol) and dried with a nitrogen flux. Then, the solid was analysed by XRPD affording TAR Form A 767 mg (98.4%). TAR Form A was also characterized by ¹H NMR, DSC, TGA and DVS.

Preparation of TAR Form A, 1 g Scale. The compound of Formula (I) (1 g) and L-tartaric acid (618.0 mg, 1.0 eq.) were suspended in EtOAc (5 mL, 5 vol). The resulting mixture was subjected to heat-cool cycles between 25° C. and 50° C. with 30 minutes at each temperature with heating and cooling of 1° C./min for 1 day. The mixture was filtered, washed with EtOAc (2×2 mL, 2 vol) and analysed wet by XRPD affording TAR Form A. Then, the solid was dried under vacuum at 25° C. overnight. An off-white solid 1.482 g (91.5%) was obtained which corresponded to TAR Form A mixed with a minor amount of TAR Form B by XRPD.

Preparation of 3-HXB Form A, 500 mg Scale. The compound of Formula (I) (507.7 mg) and 3-hydroxybenzoic acid (295.5 mg, 1.0 eq.) were suspended in EtOAc (10 mL, 20 vol). The resulting mixture was subjected to heat-cool cycles between 25° C. and 50° C. with 30 minutes at each temperature with heating and cooling of 1° C./min for 1 day. A thick suspension was obtained and EtOAc (2.5 mL, 5 vol) was added. After 1 h, the mixture was filtered, washed with EtOAc (2×1 mL, 2 vol) and analysed in wet by XRPD affording 3-HXB Form A. Then, the solid was dried under vacuum at 25° C. overnight. An off-white solid 692 mg (86.9%) was obtained which corresponded to 3-HXB Form A by XRPD. 3-HXB Form A was also characterized by ¹H NMR, DSC and TGA.

Preparation of ADI Form A in EtOAc, 1 g Scale. The compound of Formula (I) (1 g) and adipic acid (622.9 mg, 1.0 eq.) were suspended in EtOAc (10 mL, 10 vol). The resulting mixture was subjected to heat-cool cycles between 25° C. and 50° C. with 30 minutes at each temperature with heating and cooling of 1° C./min for 1 day. A thick suspension was obtained and EtOAc (2 mL, 2 vol) were added. The resulting suspension was subjected to heat-cool cycles between 25° C. and 50° C. with 30 minutes at each temperature with heating and cooling of 1° C./min for another day. The mixture was filtered, washed with EtOAc (2×2 mL, 2 vol) and analysed wet by XRPD affording ADI Form A. Then, the solid was dried under vacuum at 25° C. overnight. An off-white solid 1.247 g (79.6%) was obtained which corresponded to ADI Form A by XRPD. ADI Form A was also characterized by ¹H NMR, DSC, TGA, DVS and HPLC.

Preparation of ADI Form A in THF, 1 g Scale. The compound of Formula (I) (1 g) and adipic acid (618.4 mg, 1.0 eq.) were suspended in THF (10 mL, 10 vol). After 30 minutes a thick suspension was obtained. THF (15 mL, 15 vol) were added and the mixture was stirred at RT for 2 h. The mixture was filtered, washed with THF (2×2 mL, 2 vol) and analysed wet by XRPD affording ADI Form A. Then, the solid was dried with a nitrogen flux. An off-white solid 1.088 g (82.2%) was obtained which corresponded to ADI Form A. ADI Form A was also characterized by 1H NMR, DSC, TGA, DVS, and KF.

Preparation of GEN Form A in EtOAC. The compound of Formula (I) (1 g) and 2,5-dihydroxybenzoic acid (647.9 mg, 1.0 eq.) were suspended in EtOAc (5 mL, 5 vol). The resulting mixture was subjected to heat-cool cycles between 25° C. and 50° C. with 30 minutes at each temperature with heating and cooling of 1° C./min for 1 day. A thick suspension was obtained and EtOAc (15 mL, 15 vol) were added. After 1 h, the mixture was filtered, washed with EtOAc (2×2 mL, 2 vol) and analysed wet by XRPD affording GEN Form A. Then, the solid was dried under vacuum at 25° C. overnight. An off-white solid 1.496 g (91.6%) was obtained which corresponded to GEN Form A by XRPD. GEN Form A was also characterized by ¹H NMR, DSC, TGA, DVS and HPLC.

Preparation of GEN Form A in EtOAc, 2 g Scale. The compound of Formula (I) (2.013 g) and 2,5-dihydroxybenzoic acid (1.303 g, 1.0 eq.) were suspended in EtOAc (40 mL, 10 vol). The resulting mixture was stirred at 25° C. for 22 hours. The mixture was filtered, washed with EtOAc (2×2 vol, 4 mL) and dried under vacuum at 25° C. for 24 h. An off-white solid 3.044 g (92.5%) was obtained which corresponded to GEN Form A by XRPD.

Preparation of GEN Form A in EtOAc, 100 g Scale. The BHS salt of the compound of Formula (I) (100.5 g), sodium hydroxide (41.4 g) and water (400 mL) were introduced into a 1 L jacketed vessel and stirred for 15 minutes. The mixture was allowed to settle, and the layers were separated. The lower aqueous phase was reintroduced to the reactor with dichloromethane (200 mL) and stirred for 5 minutes. The mixture was allowed to settle, the layers separated, and the organic phases combined were separated giving a biphasic mixture. The layers were separated, and the aqueous phase extracted with dichloromethane (3×100 mL). The organic phases were combined and washed with water (100 mL) and then evaporated to dryness on a rotary evaporator. To the residue ethanol (365 mL) was added, charged to an oversized 1 L jacketed vessel, heated to 75° C. and 2,5-dihydroxybenzoic acid (36.074 g, 1.0 eq.) were added together with an additional 8 mL of ethanol washing the funnel. The solution was cooled to 68° C. and seeded with GEN Form A, 50 mg. The mixture was stirred for 1 hour at this temperature, then cooled at 0.5° C./min to 5° C. and maintained at 5° C. for 13 hours. EtOAc (640 mL) was added and the mixture stirred for 90 minutes. The mixture was filtered, washed with cold EtOAc (2×100 mL) and dried under vacuum at 50° C. for 24 h. An off-white solid 78.25 g (85.9%) was obtained.

### Additional Characterization of New Salt Forms

The new stable crystalline salt forms (ORO Form A, GEN Form A and 3-HXB Form A) were characterized by XRPD, ¹H NMR, DSC, TGA and DVS. The results are summarized in Table 35.

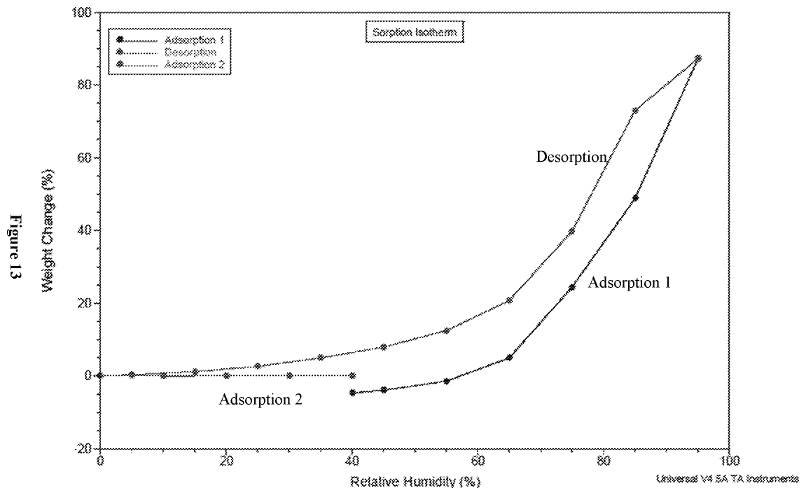

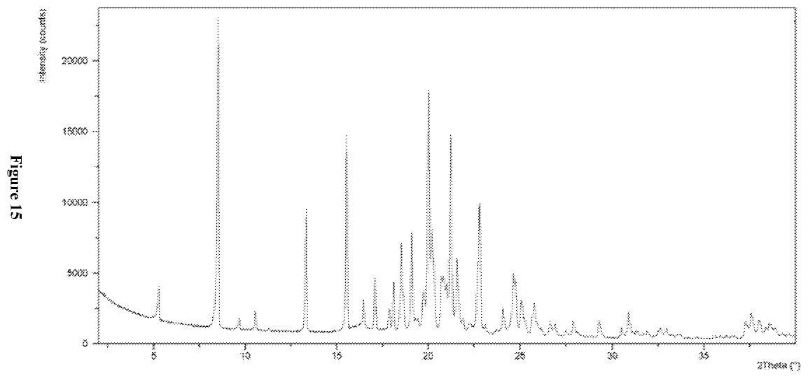

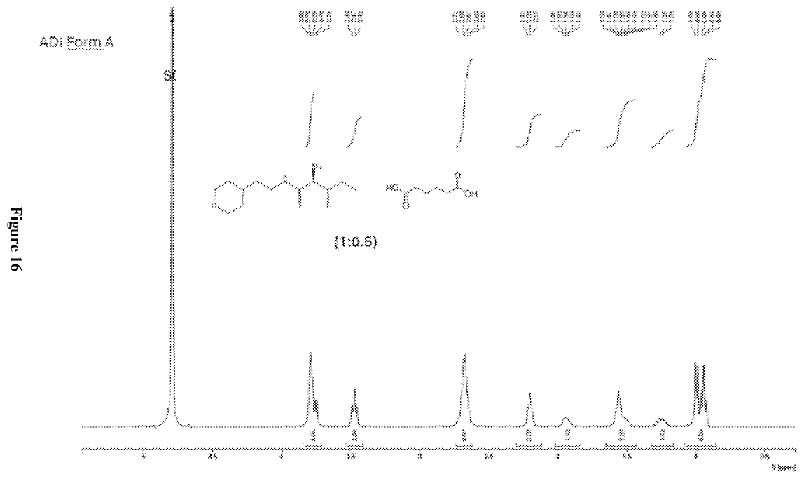

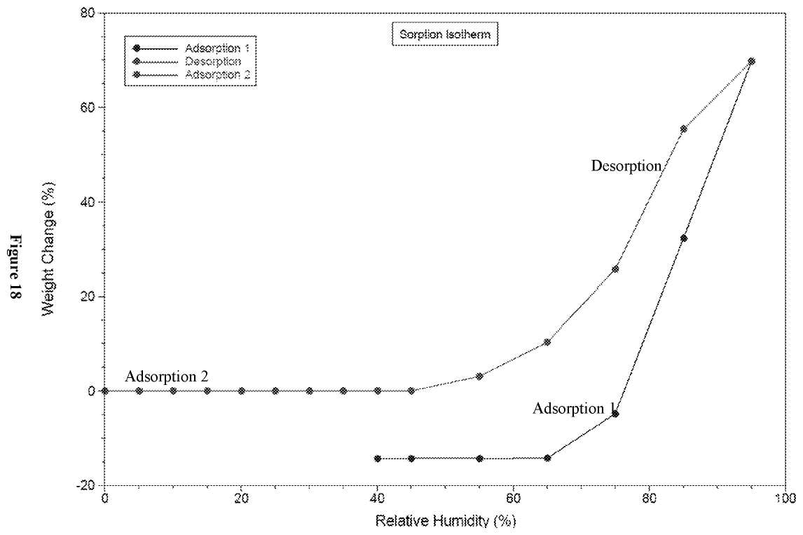

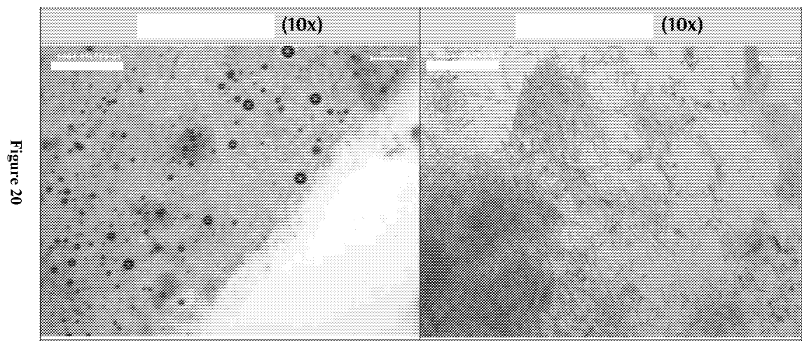

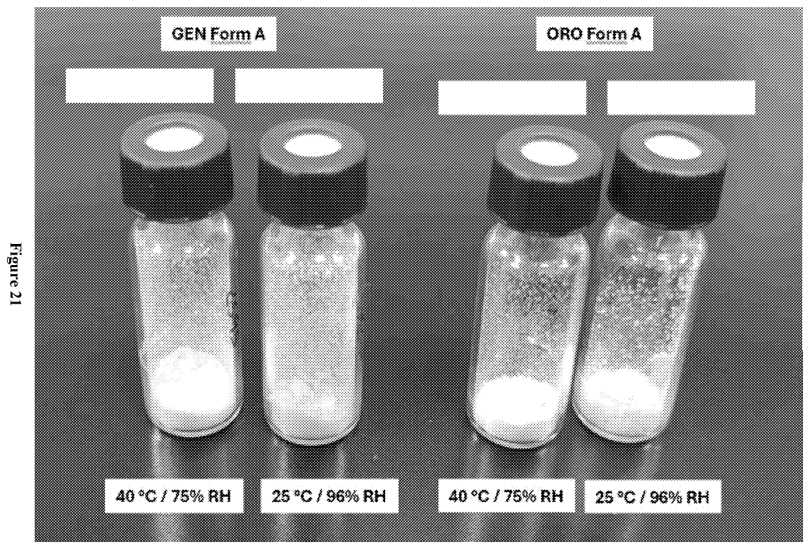

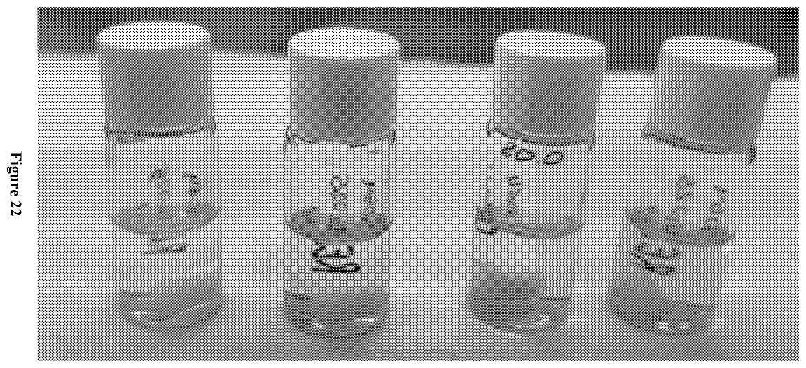

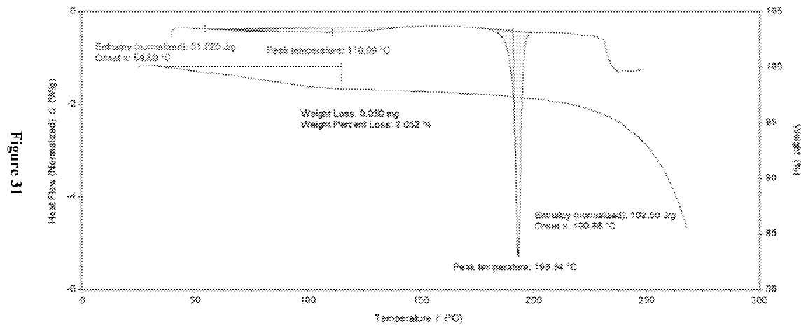

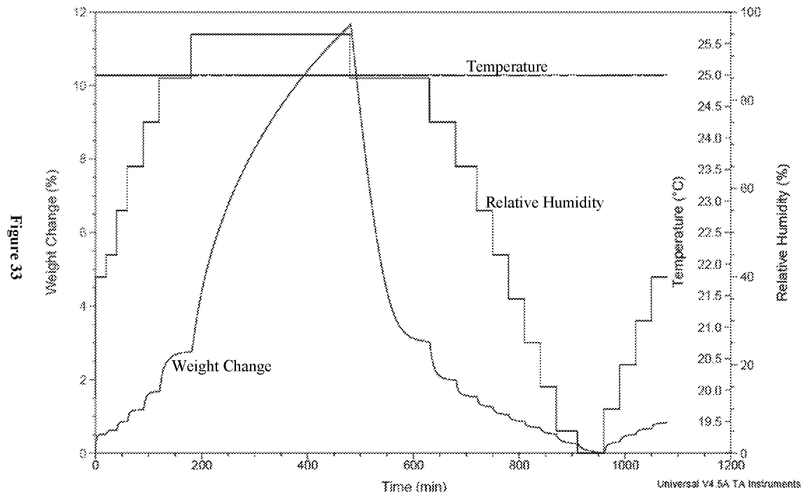

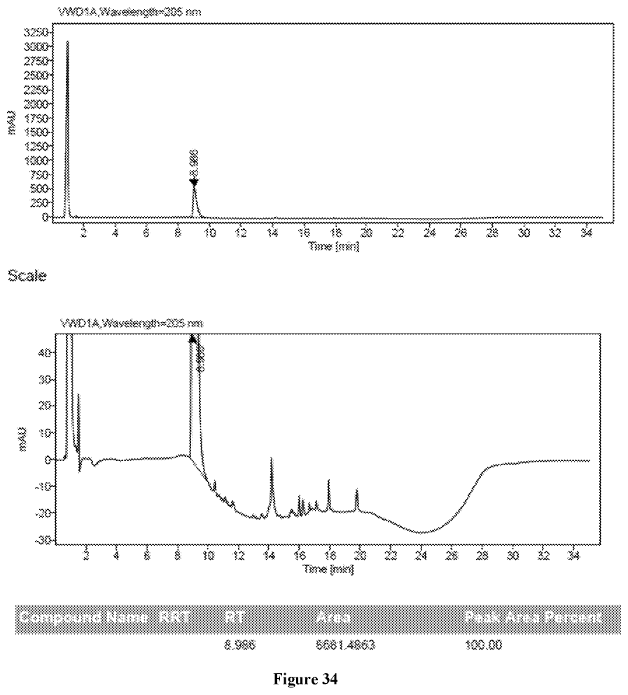

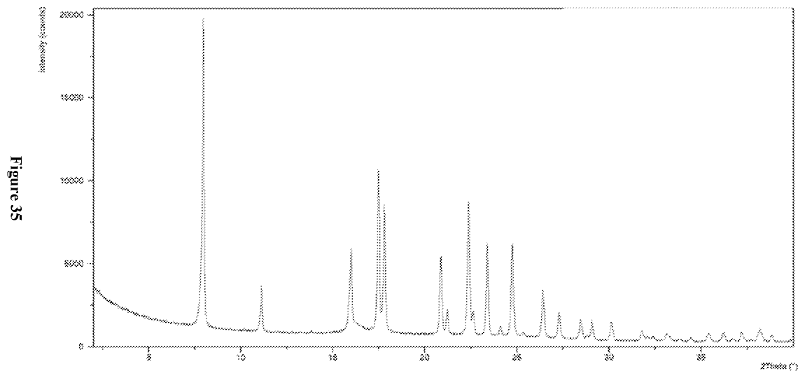

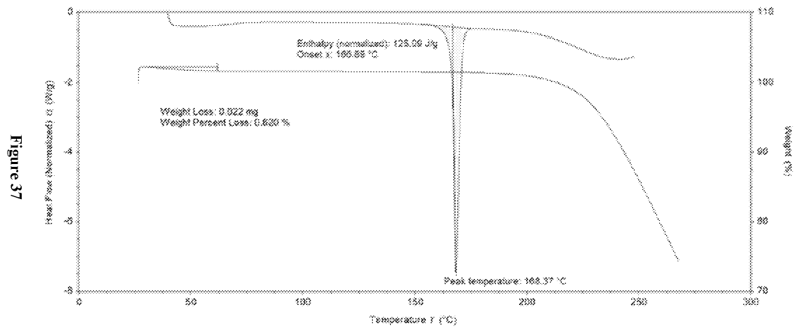

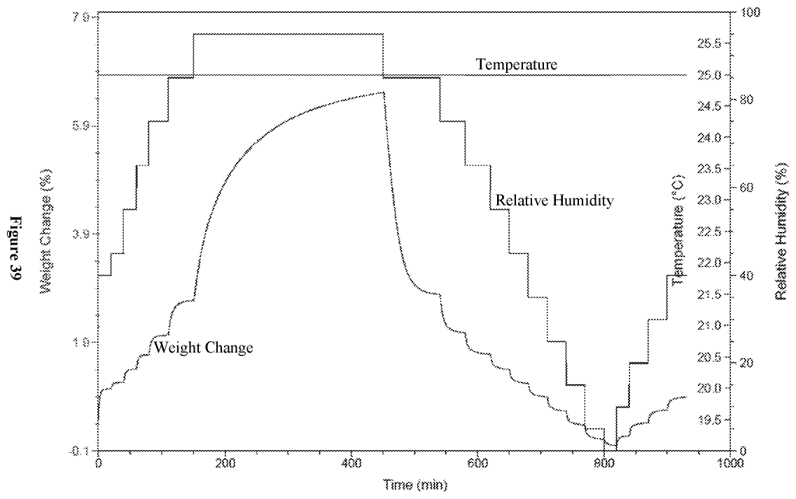

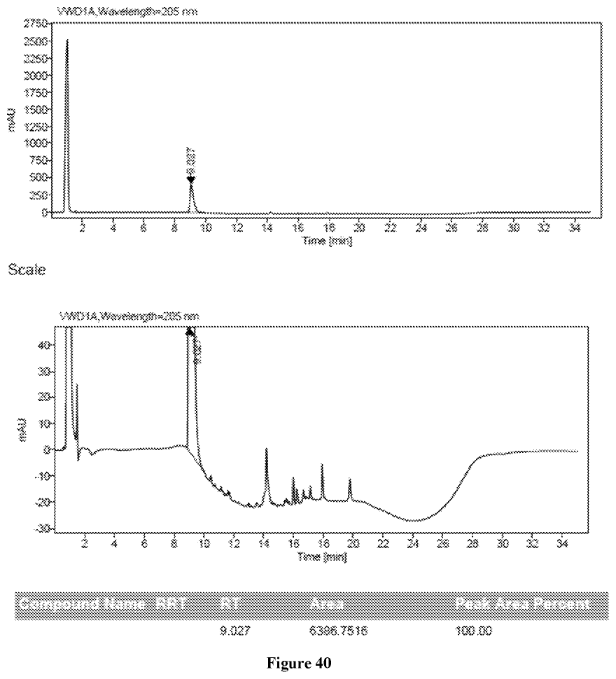

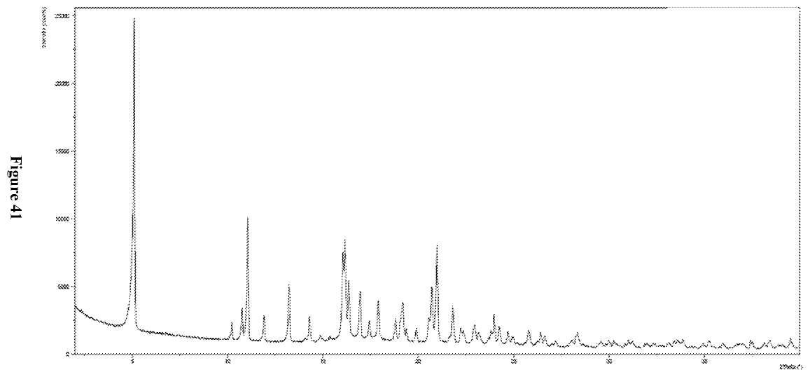

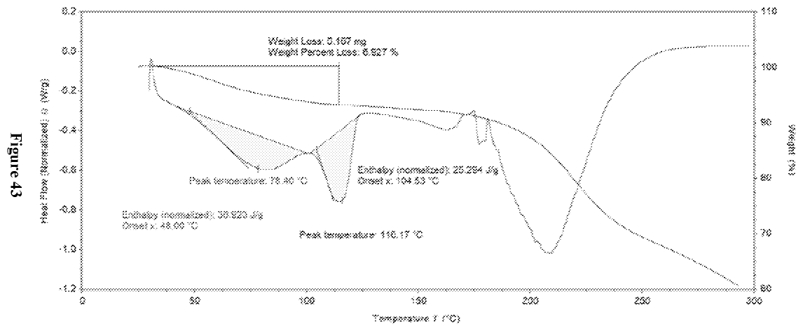

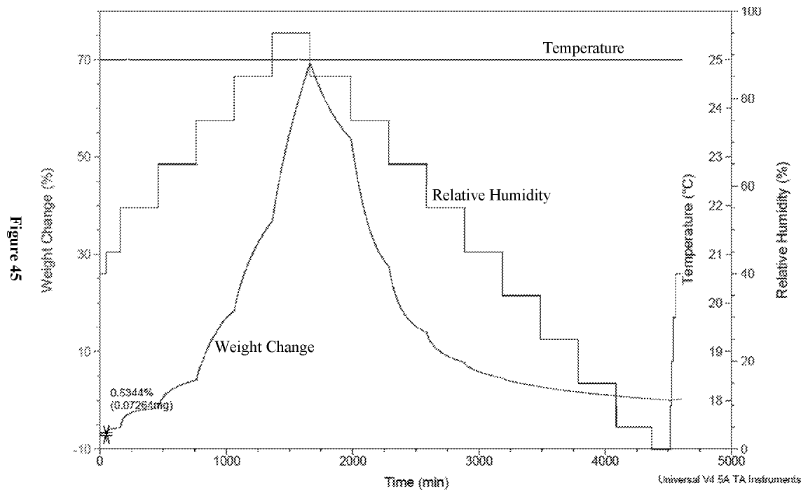

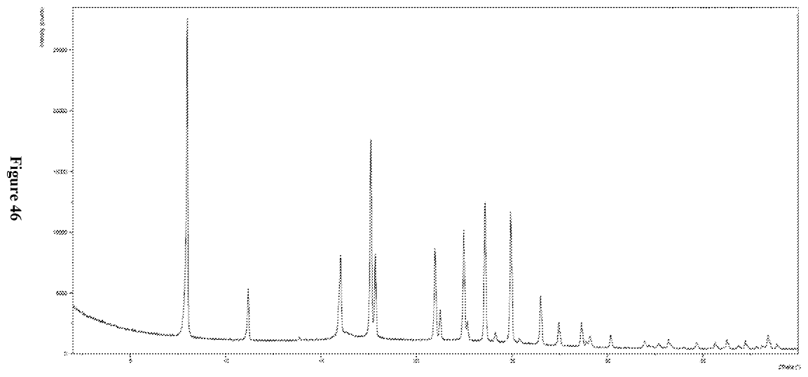

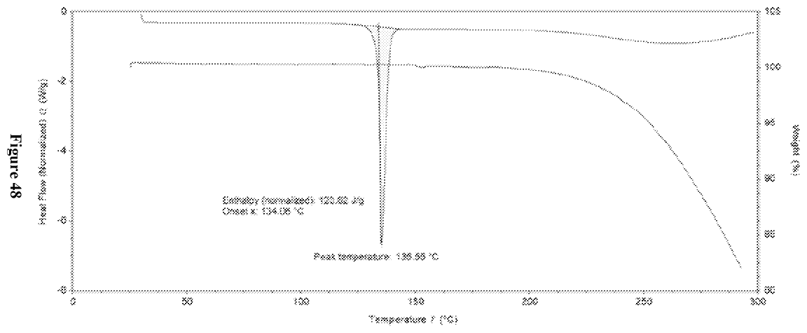

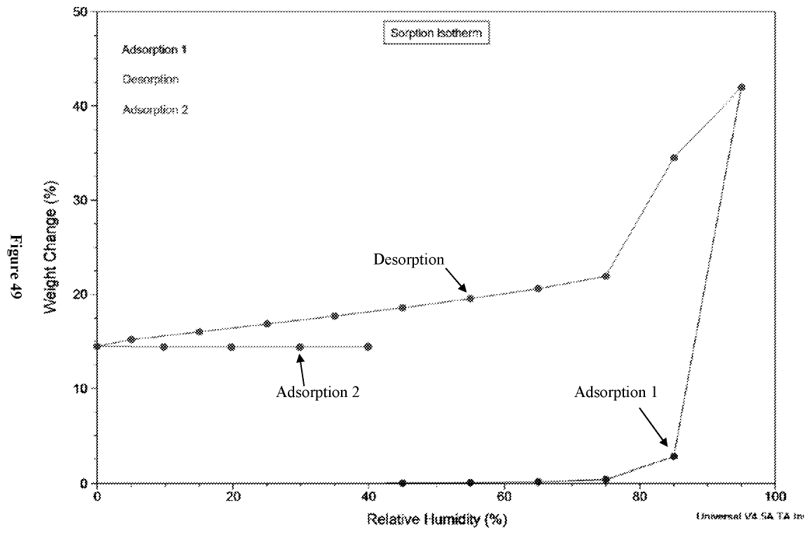

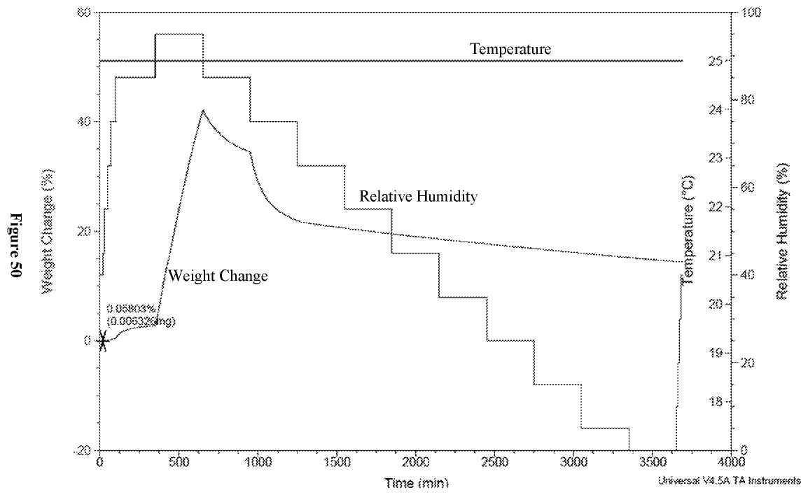

TABLE 35
Characterization Summary

| | | | | Stability at | Stability at 25° C./ | |
|---|---|---|---|---|---|---|
| Form | 1H NMR (D20) | DSC | TGA | 40° C./75% RH | 96% RH | DVS |
| ORO Form A | API:Orotic acid (1:0.7) | Broad endothermic | 2.1% loss at 30° C. to | Transforms | Transforms | 11.6% weight |
| FIG. 29 | FIG. 30 | event with onset at | 110° C. | partially | partially into ORO | change FIG. 32 |
| | | 55° C. (probably | Degradation at | into ORO | Form D after 1 day | and FIG. 33 |
| | | superficial solvent) | ~200° C. FIG. 31 | Form D | | |
| | | Melt onset at 194° C. | | after 1 day | | |
| | | FIG. 31 | | | | |
| GEN Form A | API:Gentisic acid (1:1) | Melt onset at 167° C. | 0.6% loss at 30° C. to | Stable for | Stable for 1 week | 6.5% weight |
| FIG. 35 | FIG. 36 | FIG. 37 | 60° C. Degradation at | 1 week | | change GEN |
| | | | ~200° C. FIG. 37 | | | Form A by |
| | | | | | | XRPD |
| | | | | | | FIG. 38 and |
| | | | | | | FIG. 39 |
| TAR Form A | API:L-Tartaric acid | Broad endothermic | 6.9% loss at 30° C. to | Deliquescent | Deliquescent after 1 | 69.2% weight |
| FIG. 41 | (1:1) FIG. 42 | event with onset at | 110° C. Degradation at | after 1 | day (sample holder) | change FIG. 44 |
| | | 48° C. (probably | ~180° C. FIG. 43 | day (vial) | | and FIG. 45 |
| | | superficial solvent) | | | | |
| | | Melt onset at 105° C. | | | | |
| | | FIG. 43 | | | | |
| 3-HXB Form A | API:3- | Melt onset at 134° C. | Degradation at ~200º | Stable for | Stable between 2 to | 42.0% weight |
| FIG. 46 | Hydroxybenzoic acid | FIG. 48 | C. FIG. 48 | 1 week | 7 days (sample | change FIG. 49 |
| | (1:1) FIG. 47 | | | | holder) | and FIG. 50 |
| ADI Form A in | API:Adipic acid (2:1) | Melt onset at 104° C. | 0.5% loss at 30° C. to | Stable for | Deliquescent after 2 | 87.3% weight |
| EtOAc FIG. | FIG. 11 | FIG. 12 | 65° C. Degradation at | 1 week | days (sample | change FIG. 13 |
| 10 | | | ~150° C. FIG. 12 | | holder) | and FIG. 14 |
| ADI Form A in | API:Adipic acid (2:1) | Melt onset at 104° C. | Degradation at ~150° | Stable for | Deliquescent after 2 | 84% weight |
| THF FIG. 15 | FIG. 16 | FIG. 17 | C. FIG. 17 | 1 week | days (sample | change FIG. 18 |
| | | | | | holder) | and FIG. 19 KF: |
| | | | | | | 0.41% |

### Summary of Example 3

Experiments at 1 g scale were performed successfully for ORO Form A and GEN Form A. These forms were further characterized by ¹H NMR, DSC, TGA, DVS and HPLC. The experiment performed at 1 g scale with L-tartaric acid afforded a mixture of TAR Form A and TAR Form B. GEN Form A and ORO Form A were stable in open vials after 1 week of storage at 40° C./75% RH and 25° C./96% RH by XRPD and HPLC. Slightly higher amounts of water were observed by TGA in comparison with the starting materials. Experiments at 500 mg scale were performed successfully for 3-HXB Form A, TAR Form A and ADI Form A. These forms were further characterized by ¹H NMR, DSC, TGA and DVS. ADI Form A was prepared successfully to 1 g scale in THF. The API:adipic acid ratio was 2:1 for the same prepared in EtOAc and the sample prepared in THF. Experiments at 1 g scale were performed successfully to obtain ADI Form A in THF and EtOAc. However, in EtOAc a sticky solid was obtained while in THF a fine powder was observed.

### Example 4: Further Investigation of GEN Form A, ORO Form A, TAR Form A and 3-HXB Form A

### Polymorphism Assessment of Salts

Four salts obtained during the salt screening and scaled up to 500 mg or 1 g scale (ORO Form A, GEN Form A, TAR Form A, 3-HXB Form A) were further studied by means of polymorphism assessments for each. Twelve different solvent systems were studied for each salt to provide an early insight into the propensity for polymorphism of the salts.

Procedure. The samples (20 mg±1 mg) were weighed and suspended in 5-10 volumes of the corresponding solvent. Then, mixtures were subjected to heat-cool cycles between 25° C. to 50° C. (30 min at each temperature, with heating and cooling at 1° C./min) for 2 days. Then, mixtures were centrifuged, decanted and the solids were first analysed by XRPD as wet cakes before drying under vacuum at ambient temperature and reanalysed by XRPD if a new form was obtained. The results for the polymorphism assessments for the five salts are tabulated below.

TABLE 36
Polymorphism Assessment of ORO Form A

| Ref- | | | | |
|---|---|---|---|---|
| erence | Solvent | Aspect 2 days | XRPD wet | XRPD dry |
| 1 | Water | Solution | NA | NA |
| 2 | MeCN | Suspension | ORO Form A | NP |
| 3 | EtOH | Suspension | ORO Form A + | ORO Form A + |
| | | | ORO Form D | ORO Form D |
| 4 | IPA | Suspension | ORO Form A | NP |
| 5 | Acetone | Suspension | ORO Form D | ORO Form A + |
| | | | | ORO Form D |
| 6 | MEK | Suspension | ORO Form A | NP |
| 7 | EtOAc | Suspension | ORO Form A | NP |
| 8 | i-PrOAc | Suspension | ORO Form A | NP |
| 9 | MTBE | Suspension | ORO Form A | NP |
| 10 | THF | Suspension | ORO Form A | NP |
| 11 | Toluene | Suspension | ORO Form A | NP |
| 12 | Heptane | Suspension | ORO Form A | NP |

Four different polymorphs of the orotate salt were found of which ORO Form A appears more stable. XRPD (FIG. 29), 1H NMR (FIG. 30) DSC, TGA (FIG. 31) and DVS analyses (FIG. 32 and FIG. 33) were performed. By 1H NMR the stoichiometry is 1:0.7 API:orotic acid. This form showed good crystallinity by XRPD and contains some moisture (2%) with a melt at onset 191° C. By DVS approximately 12 wt % uptake was seen, mainly above 80% RH. The adsorption-desorption process was reversible with XRPD reanalysis showing unchanged ORO Form A. ORO Form A was stable for at least 1 week at both 40° C./75% RH, 25° C./96% RH. The salt showed high solubility (>10 mg/ml) over the pH range 1.7-7.2.

TABLE 37
Polymorphism Assessment of GEN Form A

| Ref- | | | | |
|---|---|---|---|---|
| erence | Solvent | Aspect 2 days | XRPD wet | XRPD dry |
| 1 | Water | Red solution | NA | NA |
| 2 | MeCN | Suspension | GEN Form A | NP |
| 3 | EtOH | Suspension | GEN Form A | NP |
| 4 | IPA | Suspension | GEN Form A | NP |
| 5 | Acetone | Solution | NA | NA |
| 6 | MEK | Solution | NA | NA |
| 7 | EtOAc | Suspension | GEN Form A | NP |
| 8 | i-PrOAc | Suspension | GEN Form A | NP |
| 9 | MTBE | Suspension | GEN Form A | NP |
| 10 | THF | Suspension | GEN Form B | GEN Form A |
| 11 | Toluene | Suspension | GEN Form A | NP |
| 12 | Heptane | Suspension | GEN Form A | NP |

Two different polymorphs of the gentisate salt were found, GEN Form A and Form B. They appear to be related by a different degree of hydration and GEN Form A appears more stable under ambient conditions. XRPD (FIG. 35), 1H NMR (FIG. 36) DSC, TGA (FIG. 37) and DVS analyses (FIG. 38 and FIG. 39) were performed for GEN Form A. By 1H NMR the stoichiometry is 1:1 API:gentisic acid. This form showed good crystallinity by XRPD and contains some moisture (0.6%) with a melt at onset 167° C. By DVS approximately 6.5 wt % uptake was seen, mainly above 80% RH. The adsorption-desorption process was reversible with XRPD reanalysis showing unchanged GEN Form A. GEN Form A was stable for at least 1 week at both 40° C./75% RH, 25° C./96% RH. The salt showed high solubility (>10 mg/ml) over the pH range 1.7-7.2.

TABLE 38
Polymorphism Assessment of TAR Form A + TAR Form B (minor)

| Reference | Solvent | Aspect 2 days | XRPD wet | XRPD dry |
|---|---|---|---|---|
| 1 | Water | Solution | NA | NA |
| 2 | MeCN | Suspension | TAR Form A | NP |
| 3 | EtOH | Solution | NA | NA |
| 4 | IPA | Solid on the | Amorphous + | NP |
| | | walls | coformer | |
| 5 | Acetone | Suspension | TAR Form A + | NP |
| | | | coformer | |
| 6 | MEK | Suspension | TAR Form A + | NP |
| | | | coformer | |
| 7 | EtOAc | Suspension | TAR Form A + TAR | NF |
| | | | Form B | |
| 8 | i-PrOAc | Suspension | TAR Form A + TAR | NP |
| | | | Form B (minor) | |
| 9 | MTBE | Suspension | TAR Form A + TAR | NP |
| | | | Form B (minor) | |
| 10 | THF | Suspension | TAR Form C | TAR Form C + TAR |
| | | | | Form A (minor) + |
| | | | | coformer (minor) |
| 11 | Toluene | Suspension | TAR Form A + TAR | NP |
| | | | Form B (minor) | |
| 12 | Heptane | Suspension | TAR Form A + TAR | NF |
| | | | Form B | |

Three different polymorphs were identified, with TAR Form A as that most commonly obtained. XRPD (FIG. 41), 1H NMR (FIG. 42) DSC, TGA (FIG. 43) and DVS analyses (FIG. 44 and FIG. 45) were performed for TAR Form A. By 1H NMR the stoichiometry is 1:1 API:L-tartaric acid. While showing good crystallinity by XRPD, TAR Form A is hygroscopic, deliquescent and the absorption behavior in the DVS was not reversible. Additional polymorphism screening was performed using the L-tartrate salt of LM11A-BHS (TAR Form A+TAR Form B). 10 different solvent systems, together with different techniques, were used to obtain a diverse range of nucleation conditions (see Table 40).

TABLE 39
Polymorphism Assessment of 3-HXB Form A

| Ref- | | | | |
|---|---|---|---|---|
| erence | Solvent | Aspect 2 days | XRPD wet | XRPD dry |
| 1 | Water | Solution | NA | NA |
| 2 | MeCN | Suspension | 3-HXB | 3-HXB |
| | | | Form A | Form A |
| 3 | EtOH | Solution | 3-HXB | 3-HXB |
| | | | Form A | Form A |
| 4 | IPA | Suspension | 3-HXB | 3-HXB |
| | | | Form A | Form A |
| 5 | Acetone | Solution | NA | NA |
| 6 | MEK | Solution | NA | NA |
| 7 | EtOAc | Suspension | 3-HXB | 3-HXB |
| | | | Form A | Form A |
| 8 | i-PrOAc | Suspension | 3-HXB | 3-HXB |
| | | | Form A | Form A |
| 9 | MTBE | Suspension | 3-HXB | 3-HXB |
| | | | Form A | Form A |
| 10 | THF | Suspension | 3-HXB | 3-HXB |
| | | | Form A | Form A |
| 11 | Toluene | Suspension | 3-HXB | 3-HXB |
| | | | Form A | Form A |
| 12 | Heptane | Suspension | 3-HXB | 3-HXB |
| | | | Form A | Form A |

Only one polymorph of the 3-hydroxybenzoate was seen. XRPD (FIG. 46), 1H NMR (FIG. 47) DSC, TGA (FIG. 48) and DVS analyses (FIG. 49 and FIG. 50) were performed for 3-HXB Form A. By 1H NMR the stoichiometry is 1:1 API:3-hydroxybenzoic acid. This form showed good crystallinity by XRPD and is non-solvated with a melt at onset 134° C. By DVS the material was stable up to 80% RH, after which non-reversible absorption, approximately 40 wt %, was seen in the DVS, during which deliquescence occurred.

### Polymorphism Screening of TAR Form A+TAR Form B

Summary. Three different polymorphs of the L-tartrate salt (TAR Form A, TAR Form B and TAR Form C) were observed during the previous studies. However, none of them are stable at ambient conditions (25±1° C./70±5 RH) or under accelerated conditions (40° C./75% RH).

A polymorphism assessment was performed using the L-tartrate salt of the compound of Formula (I) (TAR Form A+TAR Form B). 10 different solvent systems, together with different techniques, were used to obtain a diverse range of nucleation conditions (see Table 40). A total of 20 different experiments were performed.

All the solids obtained during the polymorphism screen were analysed by XRPD as wet samples (undried). If a new crystalline form was seen, then the solid was dried under vacuum at RT and analysed once again by XRPD. Representative samples that showed a different XRPD pattern were stored in the corresponding XRPD sample holder in the humidity chamber at 40° C./75% RH with XRPD analysis periodically.

Experimental Procedures and Results. The sample (TAR Form A+TAR Form B, 25 mg±1 mg) was suspended in the corresponding solvent (250 μL) and slurried at 25° C. for 1 week. After 1 day and after 1 week aliquots were centrifuged, decanted and analysed still wet, before drying under vacuum at RT and reanalysis by XRPD if needed.

The sample (TAR Form A+TAR Form B, 20 mg±1 mg) was suspended in the corresponding solvent (200 μL) and slurried at 40° C. for 1 day. After 1 day, the mixtures were centrifuged, decanted and analysed still wet before drying under vacuum at RT and reanalysis by XRPD if needed.

TABLE 40
Summary of L-tartrate Salt Polymorphism Screening Results

| | Slurries at RT | Slurries at RT | Slurries at |
|---|---|---|---|
| Solvent | (1 d) | (7 d) | 40° C. |
| MeCN | TAR Form A | TAR Form A | TAR Form A |
| IPA | TAR Form A | TAR Form A | Amorphous |
| Acetone | Amorphous | Amorphous | TAR Form A + |
| | | | conformer |
| MEK | TAR Form A | Amorphous | TAR Form A + |
| | | | conformer |
| EtOAc | TAR Form A | TAR Form A | TAR Form A |
| i-PrOAc | TAR Form A + TAR | TAR Form A | TAR Form A |
| | Form B (minor) | | |
| MTBE | TAR Form A + TAR | TAR Form A | TAR Form A |
| | Form B (minor) | | |
| THF | TAR Form A | Amorphous | TAR Form A |
| Toluene | Solution | TAR Form A | TAR Form A |
| Nitromethane | TAR Form A | Amorphous | Amorphous |

### Stability Study of GEN Form A and ORO Form A

GEN Form A and ORO Form A were stored in open vials at 25° C./96% RH and 40° C./75% RH, monitored with dataloggers, for 2 weeks and were analysed by XRPD, TGA and HPLC every week to study their crystalline and chemical stability. A summary of the results is shown in Table 42 and Table 43. All experiments showed the same form as the starting material by XRPD after 1 week, with no evidence of chemical degradation by HPLC. Some discoloration from white or colorless to pink was seen in the gentisate salt stored at 25° C./96% RH. See FIG. 21.

TABLE 42
Stability Studies of GEN Form A and ORO Form A
in Different Conditions by XRPD and TGA

| | | | | | TGA 1 week* |
|---|---|---|---|---|---|
| | | | Aspect 1 | XRPD 1 | (% weight |
| Reference | Form | Conditions | week | week | loss) |
| 1 | GEN Form | 40° C./75% RH | Off-white | GEN Form A | 0.9 |
| | A | | solid | | |
| 2 | ORO Form | 40° C./75% RH | Off-white | ORO Form A | 2.4 |
| | A | | solid | | |
| 3 | GEN Form | 25° C./96% RH | Beige solid | GEN Form A | 0.7 |
| | A | | | | |
| 4 | ORO Form | 25° C./96% RH | Off-white | ORO Form A | 2.5 |
| | A | | solid | | |

*0.6% weight loss observed by TGA for GEN Form A at time 0. 2.0% weight loss observed for ORO Form A at time 0.

TABLE 43
Stability Studies of GEN Form A and ORO
Form A in Different Conditions by HPLC

| Ref- | | | | % Peak Area |
|---|---|---|---|---|
| erence | Starting Form | Conditions | Time Point | 1 week |
| 1 | GEN Form A | — | Time 0 | 100 |
| 2 | ORO Form A | — | Time 0 | 100 |
| 3 | GEN Form A | 25° C./96% RH | 1 week | 100 |
| 4 | ORO Form A | 25° C./96% RH | 1 week | 100 |
| 5 | GEN Form A | 40° C./75% RH | 1 week | 100 |
| 6 | ORO Form A | 40° C./75% RH | 1 week | 100 |

### Thermodynamic Solubility of GEN Form A and ORO Form A at Different pHs

The thermodynamic solubility study at pH 1.2, 4.5, 6.5 and 7.4 were performed with GEN Form A and ORO Form A. The salt, 20 mg of each, was treated with the different buffer solutions (2 mL) and stirred for 24 hours. The pH was measured after 24 hours. In case suspensions were obtained, 1 mL of each suspension was filtered with a nylon syringe filter (0.45 μm) and were diluted by a factor of 6 v/v in MeCN/water (1:1) for HPLC analysis.

TABLE 44
Thermodynamic Solubility at pH 1.2, 4.5, 6.5 and 7.4

| Conditions | Reference | XRPD Initial Solid | Observations | pH 24 h | Conc. (mg/mL) | XRPD Recovered Solid |
|---|---|---|---|---|---|---|
| pH 1.2 | 1 | GEN Form A | Solution | 1.8 | >10 | NA |
| | 2 | ORO Form A | White suspension | 1.7 | 16.2 | New form 1 |
| pH 4.5 | 3 | GEN Form A | Solution | 5.2 | >10 | NA |
| | 4 | ORO Form A | White suspension | 5.2 | 10.1 | New form 2 |
| pH 6.5 | 5 | GEN Form A | Solution | 6.6 | >10 | NA |
| pH 7.4 | 6 | ORO Form A | White suspension | 6.6 | 10.5 | New form 3 |
| | 7 | GEN Form A | Solution | 7.2 | >10 | NA |
| | 8 | ORO Form A | White suspension | 7.2 | 10.6 | New form 3 |

High solubility (>10 mg/mL) was observed in all the pH for GEN Form A. For ORO Form A, suspensions were observed in all the experiments. According to the XRPD, new crystalline forms were detected. However, the analysis by HPLC of the mother liquors afforded similar concentrations as if all the compound of Formula (I) was in solution (≈10 mg/mL). The isolated solids were analysed by HPLC and small amount of compound of Formula (I) were observed in comparison with the orotic acid peak. It is possible that the solids correspond to salts or polymorphs of orotic acid, however the content of compound of Formula (I) is low.

### Summary of Example 4

During the polymorphism assessment performed with the three salt candidates in 12 different solvents, two different polymorphs were observed with the orotate salt, the gentisate salt and the L-tartrate salt. None were stable after drying by XRPD. High solubility (>10 mg/mL) was observed for GEN Form A and ORO Form A in four different buffers (pH 1.2, 4.5, 6.5 and 7.4). No new polymorphs were observed during the polymorphism assessment of the 3-hydroxybenzoate salt and adipic acid.

### Example 5: Solubility Data of GEN Form A in Organic Solvents and Mixtures

### Evaluation

An initial evaluation of the solubility at 25° C. was conducted in different organic solvents and solvent mixtures. The solid (GEN Form A, 50 mg) was weighed in HPLC vials and 500 μL of each solvent were added. Samples were slurried at 25° C. for 24 hours, then approximately 300 μL of suspension were taken, diluted in 2 mL of MeCN/H₂O (1:1), filtered through a 0.45 μm hydrophilic syringe filter and analysed by HPLC. Experiments where solutions were obtained were not analysed by HPLC.

TABLE 45
Dissolution of GEN Form A in Different Organic
Solvents and Solvent Mixtures at 25° C.

| | | | Conc | Conc |
|---|---|---|---|---|
| Reference | Solvent | Aspect at RT | (mg/g solution) | (mg/mL solution) |
| 1 | IPA | Suspension | 8.5 | 6.7 |
| 2 | IPA:H₂O (95:5) | Solution | >126.6 | >100 |
| 3 | IPA:H₂O (90:10) | Solution | >126.6 | >100 |
| 4 | EtOAc | Suspension | 0.17 | 0.15 |
| 5 | MeOH | Solution | NA | >100 |
| 6 | EtOH | Suspension | 43 | 34 |
| 7 | EtOH:H₂O (95:5) | Solution | >126.6 | >100 |
| 8 | EtOH:H₂O (90:10) | Solution | >126.6 | >100 |
| 9 | Heptane | Suspension | NA* | NA* |

*2 phases observed when diluted in MeCN/H₂O (1:1)

### Solubility in IPA and EtOH by Determination of Clear Points

Following from the previous solubility results, IPA and EtOH were studied in more detail. The solid (GEN Form A) was weighed in HPLC vials, 0.5 mL of solvent added, and the solvent weighed for accuracy. A heating ramp from 25-80° C. at 0.5° C./min was applied and the clear point (dissolution temperature) recorded using reflectance probes. All the experiments resulted in dissolution at high temperature and the mixtures were then cooled to ambient. In most cases, spontaneous precipitation occurred during cooling. Reanalysis of the solids by XRPD showed that GEN Form A was recovered. The results are given in Table 46.

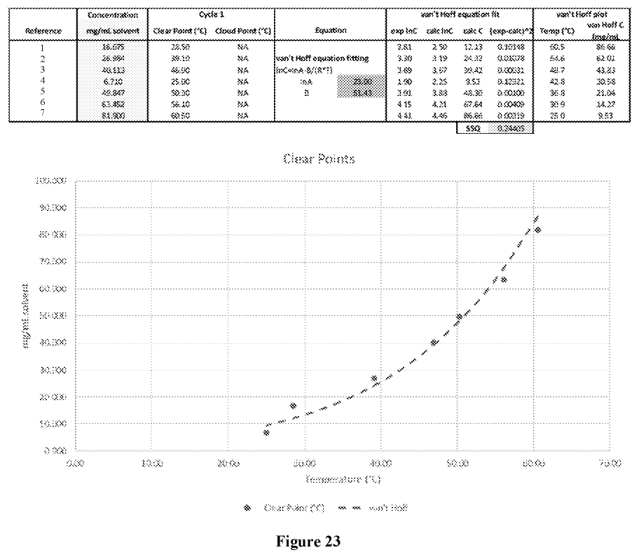

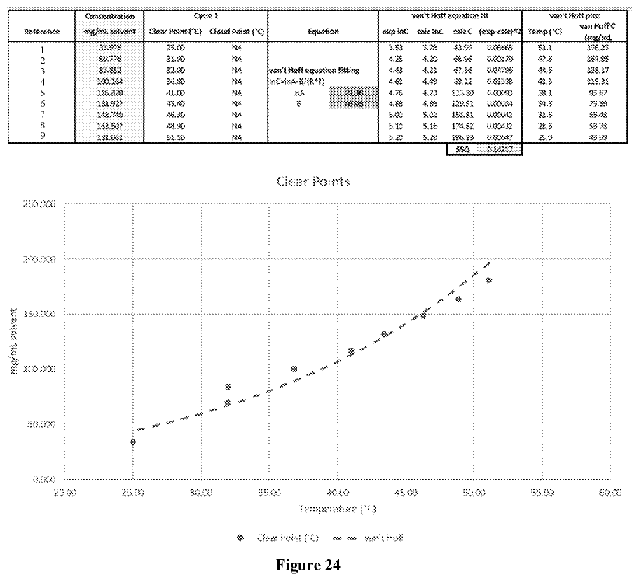

The solubility was higher in EtOH than IPA. The data was used to generate solubility curves as shown in FIG. 23 (IPA) and FIG. 24 (EtOH). The difference of solubility in IPA between high and low temperature seems promising for a cooled crystallization process. In EtOH, an antisolvent will probably be necessary to provide a high yield.

TABLE 46
Dissolution of GEN Form A in IPA and EtOH

| | | | Solvent | Conc (mg/g | Conc (mg/g | Conc | Conc | | Clear point | Aspect RT |
|---|---|---|---|---|---|---|---|---|---|---|
| Reference | Solvent | API (mg) | (mg) | solvent) | solution) | (mg/mL | (mL/mg | Aspect RT | (° C.) | after cooling |
| 1 | IPA | 5 | 381.5 | 13.1 | 12.9 | 16.7 | 60 | Suspension | 28.5 | Solution |
| 2 | IPA | 8.1 | 381.9 | 21.2 | 20.8 | 27 | 37.1 | Suspension | 39.1 | Suspension |
| 3 | IPA | 12 | 380.6 | 31.5 | 30.6 | 40.1 | 24.9 | Suspension | 46.9 | Suspension |
| 4 | IPA | 15.1 | 385.4 | 39.2 | 37.7 | 49.8 | 20.1 | Suspension | 50.3 | Suspension |
| 5 | IPA | 19.7 | 395 | 49.9 | 47.5 | 63.5 | 15.8 | Suspension | 56.1 | Suspension |
| 6 | IPA | 24.7 | 383.7 | 64.4 | 60.5 | 81.9 | 12.2 | Suspension | 60.5 | Suspension |
| 7 | EtOH | 20.1 | 365.1 | 55.1 | 52.2 | 69.8 | 14.3 | Suspension | 31.9 | Solution |
| 8 | EtOH | 25.2 | 380.9 | 66.2 | 62.1 | 83.9 | 11.9 | Suspension | 32 | Suspension |
| 9 | EtOH | 30.3 | 383.4 | 79 | 73.2 | 100.2 | 10 | Suspension | 36.8 | Suspension |
| 10 | EtOH | 35.2 | 381.9 | 92.2 | 84.4 | 116.8 | 8.6 | Suspension | 41 | Suspension |
| 11 | EtOH | 39.7 | 381.4 | 104.1 | 94.3 | 131.9 | 7.6 | Suspension | 43.4 | Suspension |
| 12 | EtOH | 45.1 | 384.3 | 117.4 | 105 | 148.7 | 6.7 | Suspension | 46.3 | Suspension |
| 13 | EtOH | 49.9 | 386.8 | 129 | 114.3 | 163.5 | 6.1 | Suspension | 48.9 | Suspension |
| 14 | EtOH | 55.1 | 385.7 | 142.9 | 125 | 181.1 | 5.5 | Suspension | 51.1 | Suspension |

### Solubility Model for GEN Form A in EtOAc-EtOH

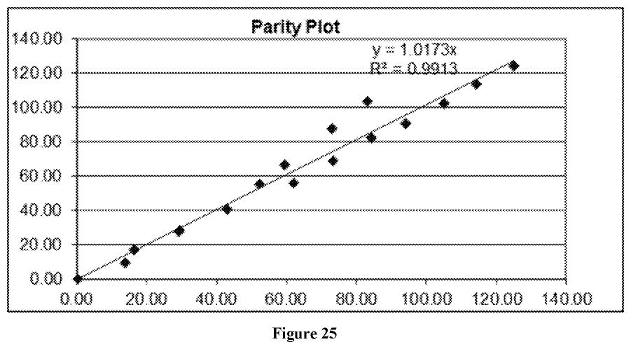

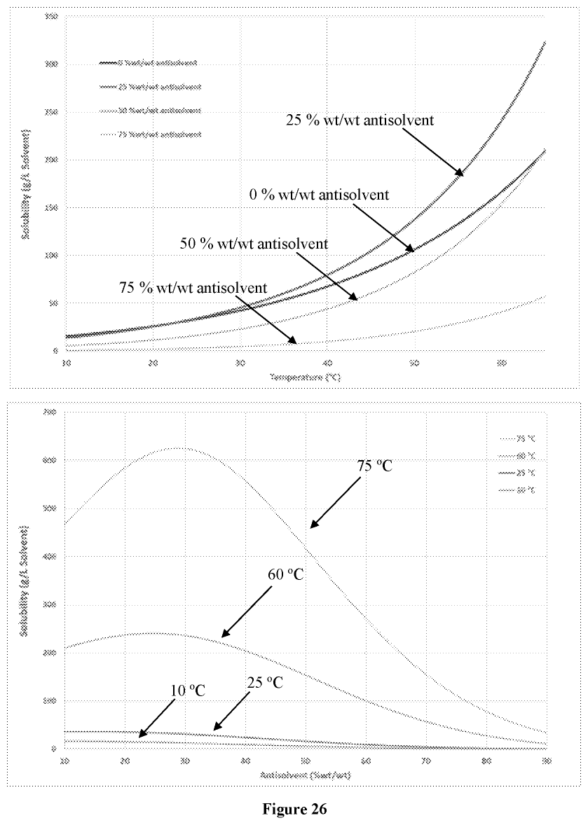

The combination of the solubility data for different experiments in EtOH, EtOAc and mixtures of both allowed the elaboration of solubility models in binary mixtures. Different models based on the van't Hoff equation were used and the constants A, B, C and D regressed against the solubility data (see Table 47). These models require the data to be expressed as g/Kg solution and x1 is the wt % fraction of antisolvent (in this case EtOAc was defined as antisolvent). The data and predictions are given in Table 47 below. The best fit was obtained in model 1, see parity plot FIG. 25, and the solubility curves are shown in FIG. 26.

TABLE 47
Van't Hoff Solubility Models

| | | | | | | | corr | |
|---|---|---|---|---|---|---|---|---|
| A | B | C | D | Equation | WSSQ | SSQ | coeff | R2 |
| 13.07026 | 22.09897 | −44.9672 | 124.0457 | C = exp(lnA0 − B0/(R * T) = | 0.391 | 4.582 | 0.9382 | 0.88 |
| | | | | −C0 * (X1) − D0 * X1/(R * T)) | | | | |
| 17.59626 | 11.83111 | −10.3479 | 22.60885 | C = exp(lnA1 − B1/(R * T) = | 0.019 | 0.288 | 0.9962 | 0.993 |
| | | | | −C1 * (X1) − D1 * exp(X1)/(R * T)) | | | | |
| −22.2027 | −37.7474 | −37.9963 | 18.9455 | C = exp(A2 * (X1\hat{ }2) + B2 * (X1)/ | 0.115 | 1.326 | 0.9825 | 0.965 |
| | | | | (R * T) + C2/(R * T) + D2 * exp(x1)) | | | | |
| 33.7936 | 51.09297 | 9.77998 | −30.34575 | C = exp(lnA3 − B3/(R * T) = | 0.155 | 1.462 | 0.9807 | 0.962 |
| | | | | −C3 * EXP(X1)− | | | | |
| −7.64334 | 7.34841 | −47.1072 | 22.4669 | C = exp(A4 * (X1\hat{ }2) + B4 * (X1)/ | 0.128 | 1.292 | 0.983 | 0.966 |
| | | | | (R * T) + C4/(R * T) + D4 | | | | |

TABLE 48
Solubility Data and Model Predictions in EtOAc-EtOH

| | | | Fraction | | | | | | | |
|---|---|---|---|---|---|---|---|---|---|---|
| | Conc g/L | | antisolv | Conc (g/Kg solution) | | Model Predictions, g/kg solution | | | | |
| Reference | solvent | Solvent | wt/wt % | Temp (° C.) | | model0 | model1 | model2 | model3 | model4 |
| 1 | 131.9 | EtOH | 0 | 43.4 | 94.28 | 107.06 | 90.94 | 90.75 | 99.47 | 96.32 |
| 2 | 148.7 | EtOH | 0 | 46.3 | 105.03 | 115.53 | 102.41 | 103.46 | 118.64 | 113.31 |
| 3 | 163.5 | EtOH | 0 | 48.9 | 114.27 | 123.56 | 113.71 | 116.12 | 138.58 | 130.76 |
| 4 | 181.1 | EtOH | 0 | 51.1 | 125 | 130.68 | 124.08 | 127.86 | 157.73 | 147.33 |
| 5 | 69.8 | EtOH | 0 | 31.9 | 52.18 | 78.01 | 55.52 | 52.66 | 47.85 | 49.05 |
| 6 | 83.9 | EtOH | 0 | 32 | 62.05 | 78.23 | 55.77 | 52.92 | 48.16 | 49.35 |
| 7 | 100.2 | EtOH | 0 | 36.8 | 73.24 | 89.53 | 68.82 | 66.73 | 65.79 | 65.79 |
| 8 | 116.8 | EtOH | 0 | 41 | 84.39 | 100.4 | 82.28 | 81.27 | 85.76 | 84.01 |
| 9 | 16 | EtOAc:EtOH | 77 | 39.9 | 13.82 | 12.25 | 9.56 | 7.3 | 7.52 | 7.46 |
| | | (74.6:25.4) | | | | | | | | |
| 10 | 19.1 | EtOAc:EtOH | 77 | 48.1 | 16.4 | 38.83 | 17.32 | 14.09 | 9.87 | 11.2 |
| | | (74.6:25.4) | | | | | | | | |
| 11 | 36 | EtOAc:EtOH | 53.1 | 33 | 29.48 | 10.89 | 28.13 | 23.49 | 30.54 | 28.15 |
| | | (49.4:50.6) | | | | | | | | |
| 12 | 35.7 | EtOAc:EtOH | 53.1 | 32.9 | 29.26 | 10.76 | 27.95 | 23.31 | 30.4 | 27.99 |
| | | (49.4:50.6) | | | | | | | | |
| 13 | 77.3 | EtOAc:EtOH | 27 | 34.8 | 59.35 | 33.07 | 66.78 | 79.68 | 68.47 | 72.64 |
| | | (24.4:75.6) | | | | | | | | |
| 14 | 96.7 | EtOAc:EtOH | 27 | 40.1 | 73.18 | 47.75 | 87.83 | 109.56 | 90.92 | 97.88 |
| | | (24.4:75.6) | | | | | | | | |
| 15 | 110.9 | EtOAc:EtOH | 27 | 43.4 | 83.02 | 59.65 | 103.68 | 132.87 | 107.95 | 117.25 |
| | | (24.4:75.6) | | | | | | | | |
| 16 | 0.15 | EtOAc | 100 | 25 | 0.17 | 0.4 | 0.2 | 0.28 | 0.31 | 0.3 |
| 17 | 33.98 | EtOH | 0 | 25 | 43.06 | 63.76 | 40.55 | 37.23 | 30.02 | 31.91 |

It is clear from the data and models that the EtOAc-EtOH system is not a simple solvent-antisolvent system and synergistic effect is seen with higher solubility at intermediate composition. The values predicted by the model in this region should be treated with caution as relatively few of the experimental data points represent the intermediate composition.

### Crystallization Experiments of GEN Form A

Following the solubility experiments, crystallization experiments with GEN Form A at 500 mg scale were performed in IPA and a mixture of EtOAc-EtOH. Results were described in Table 49.

TABLE 49
Crystallization Experiments

| | Scale | | Vol | T | Seed T | Seed | Final T | | Yield |
|---|---|---|---|---|---|---|---|---|---|
| Reference | (mg) | Solvent | (V) | (° C.) | (° C.) | age | (° C.) | XRPD | (%) |
| 1 | 500 | IPA | 13 | 75 | 67 | 30 | 25 | GEN | 77 |
| | | | | | | min | | Form A | |
| 2 | 500 | EtOAc: EtOH | 15 | 75 | 70 | 30 | 25 | GEN | 69 |
| | | (65:35) | | | | min | | Form A | |

### Summary for Example 5

Solubility curves in IPA and EtOH and a solubility model in EtOAc-EtOH were elaborated. Crystallization experiments were performed in IPA and EtOAc-EtOH affording 77% and 69% yields respectively.

### Summary of Salt/Cocrystal Studies

Salts were identified with 11 different acids: gentisic (2,5-dihydroxybenzoic acid), L-tartaric, hippuric, orotic, fumaric, propionic, adipic, mucic, 2-methoxybenzoic, 3-methoxybenzoic and 3-hydroxybenzoic acid. The salt from adipic acid was shown to be a hemi-adipate salt. All other salt forms were 1:1 API:acid. Polymorphism was observed with salts from mucic, tartaric and orotic acids. From the perspective of acceptable pharmaceutical dosing, the hemi-adipate salt may be advantageous.

Forms 2, 3 and 4 were also observed during the screening and are suspected to be polymorphs of the free base, since they were formed with different coformers. Form 2 was stable for 1 week at 40° C./75% RH, while Form 3 deliquesced under these conditions and Form 4 deliquesced at ambient temperature and humidity.

Four of the crystalline salts (TAR Form A, GEN Form A, 3-HXB Form A and ADI Form A) stored under accelerated conditions at 40° C./75% RH were stable for at least 1 week.

Four crystalline forms, hippurate, fumarate, 2-methoxybenzoate and 3-methoxybenzoate salts were deliquescent after 1 day of storage at these conditions. Similarly, the sulfate salt showed deliquescence after 1 day.

ORO Form A, ORO Form B and ORO Form C were not stable after 1 day and a new crystalline form, ORO Form D, was observed mixed with ORO Form A in all cases. These mixtures ORO Form A and ORO Form D remained stable after 1 week. It appears that ORO Forms A and D are related by different degrees of hydration.

MUC Form A transformed after 2 days into a new crystalline form, called MUC Form C, which was stable for at least 1 week at 40° C./75% RH.

The L-tartrate, gentisate, orotate, hemi-adipate and 3-hydroxybenzoate salts were characterized by 1H NMR, XRPD, DSC, TGA and DVS. The gentisate and hemi-adipate were also characterized by microscopy. The hemi-adipate exhibited fine needles whereas the particles of the gentisate were larger and blade-like. A KF analysis was performed on the hemi-adipate (0.4%).

With the exception of the adipate, orotate, and gentisate salts, the other salt forms were hygroscopic and showed non-reversible behavior in the DVS, resulting in deliquescence. The hemi-adipate salt showed little water uptake less than 70% RH, after which approximately 80 wt % moisture was absorbed.

High solubility (>10 mg/mL) was observed for GEN Form A, ORO Form A and ADI Form A in four different buffers (pH 1.2, 4.5, 6.5 and 7.4).

The gentisate and orotate salts appear to have better physical stability than the hemi-adipate, while the hemi-adipate may be advantageous from the perspective of acceptable pharmaceutical dosing. Care should be taken to store and process the hemi-adipate under controlled humidity to prevent deliquescence.

For the gentisate salt, process development was performed with solubility curves in IPA and EtOH and a solubility model in EtOAc-EtOH being elaborated to guide scale-up conditions. Three batches at 80-100 g scale were performed in 85-90% yield from EtOAc-EtOH.

In a similar way, solubility modelling of the hemi-adipate salt in 2-MeTHF and EtOAc provided conditions for scale-up. Two batches on 150 g scale in 2-MeTHF-heptane gave quantitative yield.

### Example 6: ADI Form A Scale Up and Additional Characterization

The hemi-adipate salt shows better stability under humidity conditions (40° C./75% RH) than the sulfate salt and crystalline and chemical degradation was not observed at these conditions. However, by DVS, irreversible moisture uptake was observed at high relative humidity resulting in deliquescence of the sample. Solubility modelling was performed to aid initial process development, and two batches were performed at approximately 140-150 g scale from 2-methyltetrahydrofuran-heptane in quantitative yield.

Preparation at 250 mg—1 g Scale

ADI Form A was prepared at 250 mg—1 g scale for a further characterization using the compound of Formula (I) Form 1. Heat-cool cycles between 25° C. to 50° C. (30 min at each temperature, with heating and cooling at 1° C./min) for 1 day was performed. The procedures were not optimized. The experiments are described in Table 50.

TABLE 50
Preparation of Hemi-Adipate at 250 mg - 1 g Scale

| Reference | Acid Eq. | Scale (mg) | Solvent | Vol (V) | XRPD wet | XRPD dry | Yield (%) |
|---|---|---|---|---|---|---|---|
| 1 | 1 | 250 | EtOAc | 5 | ADI Form A* | ADI Form A | 49 |
| 2 | 1 | 1000 | EtOAc | 10 + 2 | ADI Form A* | ADI Form A | 79.6 |
| 3 | 1 | 1000 | THF | 10 + 15 | ADI Form A | ADI Form A | 82.2 |
| 4 | 0.7 | 1000 | THF | 10 + 5 | ADI Form A | ADI Form A | 87.8 |

In THF the procedure gave a free flowing suspension that was easily filtered. However, when EtOAc was used, the solid was sticky after the filtration.

### Additional Characterization of ADI Form A

ADI Form A obtained from EtOAc and THF were characterized by XRPD, ¹H NMR, DSC, TGA and DVS. The results are summarized in Table 51.

TABLE 51
Characterization Summary of ADI Form A

| Solvent | EtOAc | THF |
|---|---|---|
| Aspect | Sticky solid | Free flowing solid |
| XRPD | ADI Form A FIG. 10 | ADI Form A FIG. 7 |
| ¹H NMR (D₂O) | API:Adipic acid (2:1) FIG. 11 | API:Adipic acid (2:1) FIG. 16 |
| DSC | Melt onset at 104° C. FIG. 12 | Melt onset at 104° C. FIG. 17 |
| TGA | 0.5% loss at 30° C. to 65° C. | Degradation at ~150° C. |
| | Degradation at ~150° C. FIG. 12 | FIG. 17 |
| Stability at 40° C./ | Stable for 1 week | Stable for 1 week |
| 75% RH | | |
| Stability at 25° C./ | Deliquescent after 2 days | Deliquescent after 2 days |
| 96% RH | | |
| DVS | 87.3% weight change FIG. 13 | 84% weight change FIG. 18 |
| | and FIG. 14 | and FIG. 19 |
| KF | Not analysed | 0.41% |

### Example 7: Further Investigation of ADI Form A

### Polymorphism Assessment

ADI Form A was further studied by means of polymorphism assessment. 13 different solvent systems were studied to provide an early insight into the propensity for polymorphism of ADI Form A.

Procedure. The sample (20 mg±1 mg) was weighed and suspended in 5-10 volumes of the corresponding solvent. Then, mixtures were subjected to heat-cool cycles between 25° C. to 50° C. (30 min at each temperature, with heating and cooling at 1° C./min) for 1 day. Then, mixtures were centrifuged, decanted and the solids were first analysed by XRPD as wet cakes before drying under vacuum at ambient temperature and reanalysed by XRPD if a new form was obtained.

TABLE 52
Polymorphism Assessment of ADI Form A
(from preparation in EtOAc)

| Ref- | | | | |
|---|---|---|---|---|
| erence | Solvent | Aspect 1 day | XRPD wet | XRPD dry |
| 1 | Water | Solution | NA | NA |
| 2 | MeCN | Suspension/Wax | ADI Form A | ADI Form A |
| 3 | EtOH | Solution | NA | NA |
| 4 | IPA | Solution | NA | NA |
| 5 | Acetone | Solution | NA | NA |
| 6 | MEK | Solution | NA | NA |
| 7 | EtOAc | Suspension/Wax | ADI Form A | ADI Form A |
| 8 | i-PrOAc | Suspension/Wax | ADI Form A | ADI Form A |
| 9 | MTBE | Suspension/Wax | ADI Form A | ADI Form A |
| 10 | THF | Suspension | ADI Form A | ADI Form A |
| 11 | Toluene | Suspension/Wax | ADI Form A | ADI Form A |
| 12 | Heptane | Suspension/Wax | ADI Form A | ADI Form A |

TABLE 53
Polymorphism Assessment of ADI Form A
(from preparation in THF)

| Ref- | | | | |
|---|---|---|---|---|
| erence | Solvent | Aspect 1 day | XRPD wet | XRPD dry |
| 1 | 2-MeTHF | Cotton like | ADI Form A | ADI Form A |
| 2 | MeCN | Free flowing | ADI Form A | ADI Form A |
| | | suspension | | |
| 3 | EtOH | Solution | NA | NA |
| 4 | IPA | Solution | NA | NA |
| 5 | Acetone | Solution | NA | NA |
| 6 | MEK | Solution | NA | NA |
| 7 | EtOAc | Free flowing | ADI Form A | ADI Form A |
| | | suspension | | |
| 8 | i-PrOAc | Free flowing | ADI Form A | ADI Form A |
| | | suspension | | |
| 9 | MTBE | Free flowing | ADI Form A | ADI Form A |
| | | suspension | | |
| 10 | THF | Cotton like | ADI Form A | ADI Form A |
| 11 | Toluene | Free flowing | ADI Form A | ADI Form A |
| | | suspension | | |
| 12 | Heptane | Free flowing | ADI Form A | ADI Form A |
| | | suspension | | |

Only one polymorph of the hemi-adipate salt was seen. XRPD (FIG. 15), 1H NMR (FIG. 16) DSC, TGA (FIG. 17) and DVS analyses (FIG. 18 and FIG. 19) were performed for ADI Form A. By 1H NMR the ratio of API:adipic acid is 1:0.5. This form showed good crystallinity by XRPD, is essentially non-solvated with a melt at onset 104° C. By DVS approximately 80 wt % uptake was seen, mainly above 80% RH. The adsorption-desorption process was non-reversible with deliquescence occurring during the process. ADI Form A was stable for at least 1 week at 25° C./75% RH, but deliquesced at 25° C./96% RH after 2 days. The salt showed high solubility (>10 mg/mL) over the pH range 1.7-7.2.

### Stability Study of ADI Form A

ADI Form A was stored in open vials at 25° C./60% RH and 40° C./75% RH, monitored with dataloggers, for 2 weeks and were analysed by XRPD, TGA and HPLC every week to study their crystalline and chemical stability. A summary of the results is shown in Table 54. By HPLC no impurities were seen.

TABLE 54
Stability Studies of ADI Form A in Different Conditions by XRPD and TGA

| Ref- | | | | | TGA (% |
|---|---|---|---|---|---|
| erence | Conditions | Time Point | Aspect | XRPD | weight loss) |
| 1 | — | Time 0 | Off-white solid | ADI Form A | 0 |
| 2 | 40° C./75% RH | 1 week | Off-white solid | ADI Form A | 1.9 |
| 3 | 40° C./75% RH | 2 weeks | Off-white solid | ADI Form A | 0.4 |
| 4 | 25° C./60% RH | 1 week | Off-white solid | ADI Form A | 0 |
| 5 | 25° C./60% RH | 2 weeks | Off-white solid | ADI Form A | 0 |

### Thermodynamic Solubility of ADI Form A at Different pHs

The thermodynamic solubility study at pH 1.2, 4.5, 6.5 and 7.4 was performed with ADI Form A.

Procedure. The salt, 20 mg of each, was treated with the different buffer solutions (2 mL) and stirred for 24 hours at 25° C. The pH was measured after 24 hours. In case suspensions were obtained, 1 mL of each suspension was filtered with a nylon syringe filter (0.45 μm) and were diluted by a factor of 6 v/v in MeCN/water (1:1) for HPLC analysis.

TABLE 55
Thermodynamic Solubility at pH 1.2, 4.5, 6.5 and 7.4

| | | XRPD | | | Conc. | XRPD |
|---|---|---|---|---|---|---|
| Conditions | Reference | Initial Solid | Observations | pH 24 h | (mg/mL) | Recovered Solid |
| pH 1.2 | 1 | ADI Form | Solution | 1.8 | >10 | NA |
| | | A | | | | |
| pH 4.5 | 2 | ADI Form | Solution | 5.5 | >10 | NA |
| | | A | | | | |
| pH 6.5 | 3 | ADI Form | Solution | 6.7 | >10 | NA |
| | | A | | | | |
| pH 7.4 | 4 | ADI Form | Solution | 7.2 | >10 | NA |
| | | A | | | | |

High solubility (>10 mg/mL) was observed in all aqueous buffers, from pH 1.2 to 7.4.

### Summary of Example 6

During the polymorphism assessment in 13 different solvents no new crystalline forms were detected. High solubility (>10 mg/mL) was observed for ADI Form A in four different buffers (pH 1.2, 4.5, 6.5 and 7.4).

### Example 8: Solubility Data of ADI Form A in Organic Solvents and Mixtures

### Evaluation

An initial evaluation of the solubility at 25° C. was conducted in different organic solvents. Solvents were chosen on the basis of ICH class, looking for both high (solvent) and low (antisolvent) solubilizing potential. The solid (ADI Form A, 50 mg) was weighed in HPLC vials and 500 μL of each solvent were added. Samples were slurried at 25° C. for 24 hours, then approximately 300 μL of suspension were taken, diluted in 2 mL of MeCN/H₂O (1:1), filtered through a 0.45 μm hydrophilic syringe filter and analysed by HPLC. Experiments where solutions were obtained were not analysed by HPLC.

TABLE 56
Dissolution of ADI Form A in Different Organic Solvents at 25° C.

| | | | | Conc | Conc |
|---|---|---|---|---|---|
| Ref- | | ICH | Aspect | (mg/g | (mg/mL |
| erence | Solvent | Class | at RT | solution) | solution) |
| 1 | 2-MeTHF | 3 | Suspension | 19 | 16.2 |
| 2 | MeCN | 2 | Suspension | 13.7 | 10.8 |
| 3 | EtOH | 3 | Solution | >126.6 | >100 |
| 4 | IPA | 3 | Solution | >126.6 | >100 |
| 5 | Acetone | 3 | Solution | >126.6 | >100 |
| 6 | MEK | 3 | Solution | >126.6 | >100 |
| 7 | EtOAc | 3 | Suspension | 6 | 5.3 |
| 8 | i-PrOAc | 3 | Suspension | 57.3 | 49.9 |
| 9 | MTBE | 3 | Suspension | 2.3 | 1.7 |
| 10 | THF | 2 | Suspension | 34 | 30.2 |
| 11 | Toluene | 2 | Suspension | NA* | NA* |
| 12 | Heptane | 3 | Suspension | NA* | NA* |

*2 phases observed when diluted in MeCN/H₂O (1:1)

Solubility in 2-MeTHF, iPrOAc, and EtOAc by Determination of Clear Points

Following from the previous solubility results, 2-MeTHF, iPrOAc, and EtOAc were studied in more detail. The solid (ADI Form A) was weighed in HPLC vials, 0.5 mL of solvent added, and the solvent weighed for accuracy. A heating ramp from 25-80° C. at 0.5° C./min was applied and the clear point (dissolution temperature) recorded using reflectance probes. All the experiments resulted in dissolution at high temperature and the mixtures were then cooled to 25° C. at 0.5° C./min. In all cases, spontaneous precipitation occurred during cooling. Reanalysis of the solids by XRPD showed that ADI Form A was recovered. The results are given in Table 57.

TABLE 57
Dissolution of ADI Form A in 2-MeTHF, iPrOAc, and EtOAc

| | | | | Conc | Conc | Conc | Conc | Clear | Cloud |
|---|---|---|---|---|---|---|---|---|---|
| | | API | Solvent | (mg/g | (mg/g | (mg/mL | (mL/mg | point | point |
| Reference | Solvent | (mg) | (mg) | solvent) | solution) | solvent) | solvent) | (° C.) | (° C.) |
| 1 | 2−MeTHF | 14.9 | 400.7 | 37.2 | 35.9 | 43.5 | 23 | 43.2 | 25.2 |
| 2 | 2−MeTHF | 19.4 | 421 | 46.1 | 44.1 | 54 | 18.5 | 46.1 | 39 |
| 3 | 2−MeTHF | 25.8 | 425.8 | 60.6 | 57.1 | 71 | 14.1 | 50.8 | 37.5 |
| 4 | 2−MeTHF | 35.7 | 414.3 | 86.2 | 79.3 | 100.9 | 9.9 | 53.9 | 50.4 |
| 5 | 2−MeTHF | 41.8 | 418.4 | 99.9 | 90.8 | 117 | 8.5 | 57.8 | 45.7 |
| 6 | 2−MeTHF | 44.9 | 417.9 | 107.4 | 97 | 125.8 | 7.9 | 60 | 55.2 |
| 7 | 2−MeTHF | 49.8 | 425.1 | 117.1 | 104.9 | 137.2 | 7.3 | 61.3 | 54.3 |
| 8 | 2−MeTHF | 54.3 | 425.5 | 127.6 | 113.2 | 149.4 | 6.7 | 62.7 | 44.3 |
| 9 | 2−MeTHF | 59.2 | 425.3 | 139.2 | 122.2 | 163 | 6.1 | 62.8 | 55.5 |
| 10 | iPrOAc | 30.6 | 415.8 | 73.6 | 68.5 | 84.6 | 11.8 | 64.2 | 62.8 |
| 11 | iPrOAc | 34.5 | 426.5 | 80.9 | 74.8 | 93 | 10.8 | 64.6 | 65.9 |
| 12 | iPrOAc | 40.7 | 425.8 | 95.6 | 87.2 | 109.9 | 9.1 | 64.8 | 67.5 |
| 13 | iPrOAc | 57.8 | 426.2 | 135.6 | 119.4 | 155.9 | 6.4 | 68.7 | 70.2 |
| 14 | EtOAc | 5.5 | 440.9 | 12.5 | 12.3 | 13.8 | 72.3 | 38.9 | 26.3 |
| 15 | EtOAc | 10 | 427.4 | 23.4 | 22.9 | 25.9 | 38.6 | 49.6 | 38.4 |
| 16 | EtOAc | 15.2 | 443.3 | 34.3 | 33.2 | 38 | 26.3 | 53.9 | 30.6 |
| 17 | EtOAc | 19.7 | 444.2 | 44.3 | 42.5 | 49.2 | 20.3 | 57.6 | 47.5 |
| 18 | EtOAc | 24.9 | 446.3 | 55.8 | 52.8 | 61.9 | 16.2 | 58.5 | 52.4 |
| 19 | EtOAc | 30 | 444.1 | 67.6 | 63.3 | 74.9 | 13.4 | 62 | 50 |
| 20 | EtOAc | 35.9 | 450.6 | 79.7 | 73.8 | 88.3 | 11.3 | 61.3 | 56.2 |

In 2-MeTHF, a good difference in solubility at high temperature (59° C., 163 mg/mL) vs low (25° C., 16 mg/mL) was observed while in iPrOAc the salt has a similar solubility to 2-MeTHF at high temperature (58° C., 156 mg/mL), but higher solubility at low temperature (25° C., 50 mg/mL). A wider temperature range is potentially offered by iPrOAc. However, the higher solubility at low temperatures favors 2-MeTHF for increased yield, potentially without the need for antisolvent.

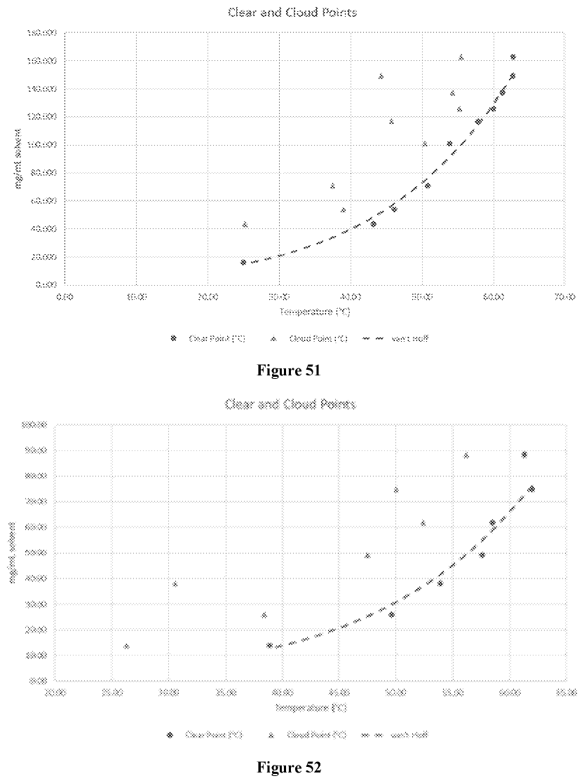

The solubility data for 2-MeTHF is represented in FIG. 51. The clear points, together with HPLC data at 25° C., provide the solubility curve. Together with the cloud points we get an idea of the meta-stable zone. Significant variability is seen in the cloud points, which is not uncommon. The MSZ width under these conditions (2-MeTHF, cooling at 5° C./min) is about 5-10° C.

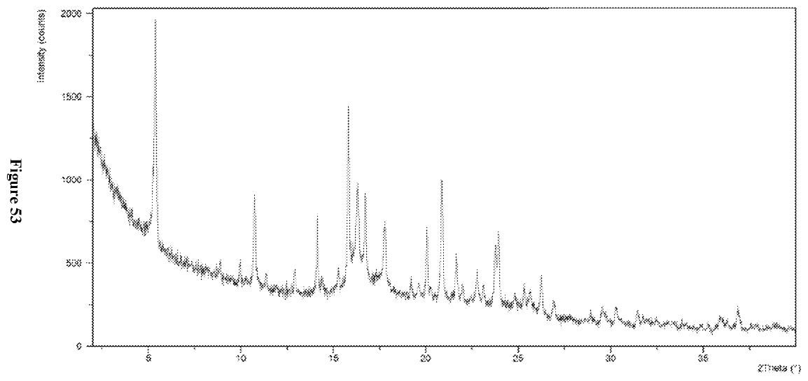

The solubility data for EtOAc is represented above in FIG. 52. The clear points, together with HPLC data at 25° C., provide the solubility curve. Together with the cloud points we get an idea of the meta-stable zone. Significant variability is seen in the cloud points, which is not uncommon. The MSZ width under these conditions (EtOAc, cooling at 5° C./min) is about 10-15° C.

### Example 9: ADI Form A Scale Up

After an initial small-scale trial at 2 g scale, the process (starting from the sulfate salt) was scaled up first to 20 g and then to 200 g batches in a 2-L jacketed vessel (see Table 58). Approximately 280 g of hemi-adipate salt will be prepared from the sulfate in 2 batches, first cracking the sulfate with NaOH and partitioning in DCM-water, concentrating the organic and crystallizing the hemi-adipate in 2-MeTHF (6 vol) with seeding at 70° C., followed by cooling to 20° C. and antisolvent addition (heptane, 2.5 vol). The experimental procedures of the two big scale-ups are described below.

TABLE 58
Scale-up Batches, Hemi-Adipate Salt

| | | | 1H NMR | | | |
|---|---|---|---|---|---|---|
| Reference | Yield (g, %) | XRPD | (DMSO) | DSC | TGA | Microscopy |
| 1 | 13.38 g, 92.7% | ADI Form | NF | Melt with onset | No weight | NP |
| | | A | | at 104° C. | loss | |
| 2 | 146.1 g, 101% | ADI Form | 2:1 ratio | Melt with onset | No weight | Small needle |
| | | A | | at 102.3° C. | loss | shape particles |
| 3 | 143.7 g, 99.7% | ADI Form | 2:1 ratio | Melt with onset | No weight | Small needle |
| | | A | | at 102.3° C. | loss | shape particles |

### Preparation of ADI Form A

The synthesis of ADI Form A began with either the free base of the compound of Formula (I), or a salt, such as the BHS salt, of the compound of Formula (I). Starting from a salt of the compound of Formula (I), the free base was liberated by treating with a base, such as with sodium hydroxide. The resulting free base was separated and then treated with adipic acid, resulting in the adipate salt. Alternatively, starting from the free base of the compound of Formula (I), the free base was treated with adipic acid, resulting in the adipate salt. A solution of the adipate salt of Formula (I) at an elevated temperature was cooled, optionally with concurrent stirring and optionally with seeding by another sample of ADI Form A, to form ADI Form A by crystallization. The product was characterized by several known methods, e.g., XRPD, DSC, TGA, and parameters were varied to obtain various distributions of particle size, e.g., FIG. 71, Table 59, while maintaining the same crystalline form.

TABLE 59
Example Particle Size Distribution

| Dv (10) (μm) | Dv (50) (μm) | Dv (90) (μm) |
|---|---|---|
| 4.43 | 30.7 | 624 |

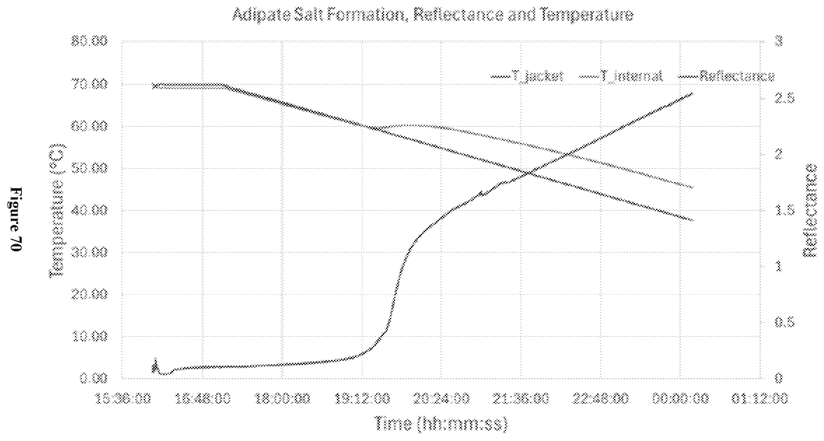

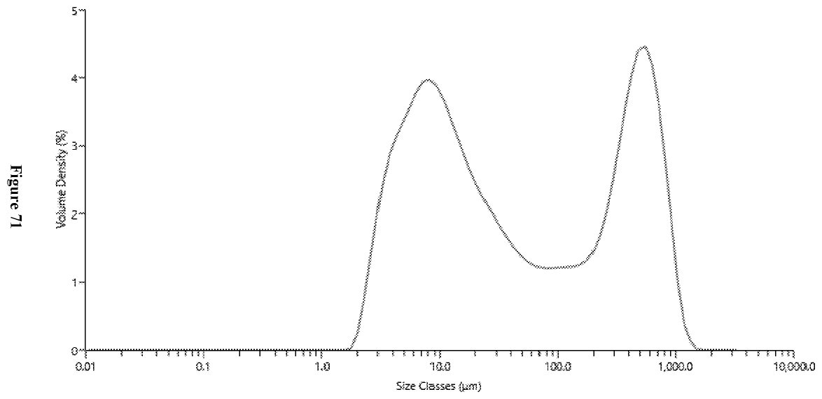

For example, the BHS salt of the compound of Formula (I) (200.3 g), sodium hydroxide (78.8 g), water (600 mL) and dichloromethane (200 mL) were introduced into a 2 L jacketed vessel and stirred for 20 minutes. The mixture was allowed to settle, the layers were separated and the aqueous phase extracted with dichloromethane (4×100 mL). The organic phases were combined and evaporated to dryness on a rotary evaporator. To the residue 2-MeTHF (800 mL, 6 V) was added, charged to a 2 L jacketed vessel, heated to 50° C. and adipic acid (33.75 g, 0.5 eq.) were added together with an additional 64 mL of 2-MeTHF washing the funnel. A suspension was immediately observed. The mixture was heated to 75° C. and a solution was observed. The solution was cooled to 70° C. and seeded with ADI Form A, 865 mg. The mixture was stirred for 1 hour at this temperature, then cooled to 20° C. in 10 hours and maintained at 20° C. for 13 hours. Heptane (432 mL, 2.5 V) was added and the mixture stirred for 30 minutes. The mixture was filtered, washed with heptane (432 mL) and dried under vacuum at 50° C. for 24 h and 30° C. for 3 days. An off-white solid 146.1 g (101%) was obtained. The temperature and reflectance profile (FIG. 69) was obtained.

### Summary of ADI Form A Studies

Two batches of ADI Form A were obtained at 1 g scale from EtOAc and THF. The XRPDs are almost identical.

No new polymorphs were observed for ADI Form A in the different solvents tested while it shows good stability by XRPD and HPLC in the conditions tested (40° C./75% RH and 25° C./60% RH) and very high solubility in different pH buffers (>10 mg/mL).

2-MeTHF may be a useful solvent for the hemi-adipate preparation at a higher scale for several reasons: (1) ICH class 3; (2) good solubility profile; and (3) immiscible with water to potentially use for extractions of the free base precursor.

The crystalline salt with adipic acid (ADI Form A) was stable for at least 1 week at 40° C./75% RH while at 25° C./96% RH deliquescence was observed after 2 days. Experiments at 1 g scale were performed successfully to obtain ADI Form A in THF and EtOAc. However, in EtOAc a sticky solid was obtained while in THF a fine powder was observed. During the polymorphism assessment in 13 different solvents no new crystalline forms were detected. High solubility (>10 mg/mL) was observed for ADI Form A in four different buffers (pH 1.2, 4.5, 6.5 and 7.4). No crystalline or chemical degradation was detected by XRPD or HPLC at different conditions. Solubility curves in 2-MeTHF and EtOAc were elaborated. Two batches at 200 g scale were prepared by crystallization in 2-MeTHF-heptane.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1-56. (canceled)

57. A compound, wherein the compound is a salt of a compound of Formula (I): 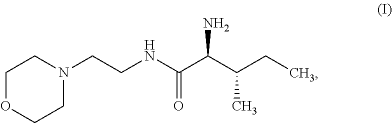 [SMILES: CC[C@H](C)[C@H](N)C(=O)NCCN1CCOCC1]
wherein the salt is an adipic salt.

58. The compound of claim 57, wherein the adipic salt is:

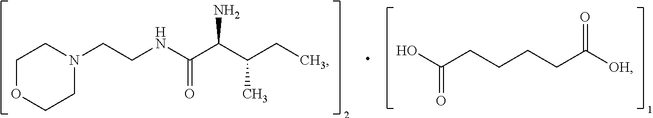

59. The compound of claim 58, wherein the compound is in a crystalline form.

60. The compound of claim 59, wherein the crystalline form exhibits an X-ray powder diffraction pattern comprising peaks at the following diffraction angles (2θ): 8.4±0.2, 15.4±0.2, 19.8±0.2, 21.0±0.2, and 22.6±0.2.

61. The compound of claim 60, wherein the crystalline form exhibits an X-ray powder diffraction pattern comprising peaks at the following diffraction angles (2θ): 8.4±0.2, 13.2±0.2, 15.4±0.2, 18.3±0.2, 18.9±0.2, 19.8±0.2, 21.0±0.2, 21.4±0.2, 22.6±0.2, and 24.4±0.2.

62. The compound of claim 60, wherein the crystalline form exhibits an X-ray powder diffraction pattern substantially the same as depicted in FIG. 10 or FIG. 15.

63. The compound of claim 60, wherein the crystalline form has a melting point onset as determined by differential scanning calorimetry at about 104° C.

64. The compound of claim 60, wherein the crystalline form has a differential scanning calorimetry curve substantially the same as shown in FIG. 12 or FIG. 17.

65. A pharmaceutical composition comprising the compound of claim 57, and a pharmaceutically acceptable excipient.

66. A method of treating a disease involving degradation, degeneration or dysfunction of cells expressing p75, the method comprising administering to the subject a therapeutically effective amount of the compound of claim 57.

67. A compound, wherein the compound is a salt of a compound of Formula (I): 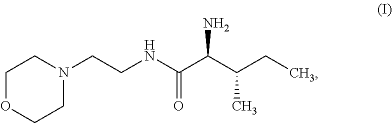 [SMILES: CC[C@H](C)[C@H](N)C(=O)NCCN1CCOCC1]
wherein the salt is a gentisic salt.

68. The compound of claim 67, wherein the gentisic salt is: 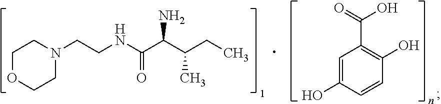
wherein n is 1.

69. The compound of claim 68, wherein the compound is in a crystalline form.

70. The compound of claim 69, wherein the crystalline form exhibits an X-ray powder diffraction pattern comprising peaks at the following diffraction angles (2θ): 7.9±0.2, 17.3±0.2, 22.1±0.2, 23.1±0.2, and 24.5±0.2.

71. The compound of claim 70, wherein the crystalline form exhibits an X-ray powder diffraction pattern comprising peaks at the following diffraction angles (2θ): 7.9±0.2, 11.0±0.2, 15.8±0.2, 17.3±0.2, 17.6±0.2, 20.6±0.2, 22.1±0.2, 23.1±0.2, 24.5±0.2, and 26.1±0.2.

72. The compound of claim 70, wherein the crystalline form exhibits an X-ray powder diffraction pattern substantially the same as depicted in FIG. 35.

73. The compound of claim 70, wherein the crystalline form has a melting point onset as determined by differential scanning calorimetry at about 167° C.

74. The compound of claim 70, wherein the crystalline form has a differential scanning calorimetry curve substantially the same as shown in FIG. 37.

75. A pharmaceutical composition comprising the compound of claim 67 and a pharmaceutically acceptable excipient.

76. A method of treating a disease involving degradation, degeneration or dysfunction of cells expressing p75, the method comprising administering to the subject a therapeutically effective amount of the compound of claim 67.

77. A compound, wherein the compound is a salt of a compound of Formula (I): 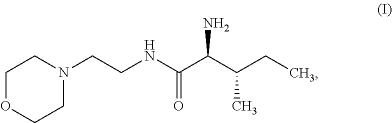 [SMILES: CC[C@H](C)[C@H](N)C(=O)NCCN1CCOCC1]
wherein the salt is an orotic salt.

78. The compound of claim 77, wherein the orotic salt is: 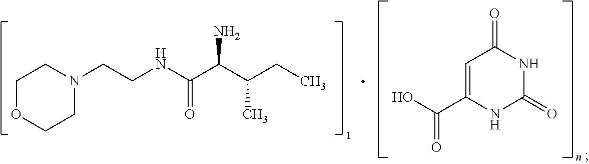
wherein n is 0.7.

79. The compound of claim 78, wherein the compound is in a crystalline form.

80. The compound of claim 79, wherein the crystalline form exhibits an X-ray powder diffraction pattern comprising peaks at the following diffraction angles (2θ): 4.7±0.2, 14.4±0.2, 14.6±0.2, 15.9±0.2, and 27.3±0.2.

81. The compound of claim 80, wherein the crystalline form exhibits an X-ray powder diffraction pattern comprising peaks at the following diffraction angles (2θ): 4.7±0.2, 14.4±0.2, 14.6±0.2, 15.9±0.2, 16.1±0.2, 18.8±0.2, 20.6±0.2, 22.2±0.2, 26.9±0.2, and 27.3±0.2.

82. The compound of claim 80, wherein the crystalline form exhibits an X-ray powder diffraction pattern substantially the same as depicted in FIG. 29.

83. The compound of claim 80, wherein the crystalline form has a melting point onset as determined by differential scanning calorimetry at about 191° C.

84. The compound of claim 80, wherein the crystalline form has a differential scanning calorimetry curve substantially the same as shown in FIG. 31.

85. A pharmaceutical composition comprising the compound of claim 77, and a pharmaceutically acceptable excipient.

86. A method of treating a disease involving degradation, degeneration or dysfunction of cells expressing p75, the method comprising administering to the subject a therapeutically effective amount of the compound of claim 77.
